(19) United States
(12) Patent Application Publication
(10) Pub. No.: US 2026/0263188 A1
(43) Pub. Date: Sep. 10, 2026

(54) BIORESORBABLE TAGS FOR MONITORING DEEP TISSUE HOMEOSTASIS

(71) Applicants: NORTHWESTERN UNIVERSITY, Evanston, IL (US); WASHINGTON UNIVERSITY, St. Louis, MO (US)

(72) Inventors: John A. Rogers, Wilmette, IL (US); Jiaqi Liu, Evanston, IL (US); Chet Hammill, St. Louis, MO (US); Matthew R. MacEwan, St. Louis, MO (US); Naijia Liu, Evanston, IL (US); Yameng Xu, St. Louis, MO (US); Mingzheng Wu, Evanston, IL (US); Haohui Zhang, Evanston, IL (US); Yonggang Huang, Evanston, IL (US); Yima He, Evenston, IL (US); Toe Yeon Kim, Evanston, IL (US)

(21) Appl. No.: 19/560,710

(22) Filed: Mar. 9, 2026

Related U.S. Application Data
(60) Provisional application No. 63/768,246, filed on Mar. 7, 2025.

(51) Int. Cl. A61B 90/00; A61B 8/00; A61B 8/08; A61L 31/02; A61L 31/04; A61L 31/14; A61L 31/18

(52) U.S. Cl. CPC A61B 90/39; A61B 8/08; A61B 8/481; A61B 8/5223; A61L 31/022; A61L 31/048; A61L 31/14; A61L 31/145; A61L 31/148; A61L 31/18; A61B 2090/3925; A61B 2090/3995; A61B 2503/42

(57) ABSTRACT

This invention relates to a bioresorbable, shape-adaptive materials structure that enables real-time monitoring of deep-tissue homeostasis using conventional ultrasound, CT or MRI instruments. Collections of small, bioresorbable metal discs distributed within thin, pH-responsive hydrogels, deployed by surgical implantation or syringe injection, allow ultrasound, CT or MRI based measurements of spatio-temporal changes in pH for early assessments of anastomotic leaks following gastrointestinal surgeries, where bioresorption after a recovery period eliminates the need for surgical extraction. Demonstrations in small and large animal models illustrate capabilities in monitoring leakage from the small intestine, the stomach, and the pancreas.

### CROSS-REFERENCE TO RELATED PATENT APPLICATIONS

This application claims priority to and the benefit of U.S. Provisional Application No. 63/768,246, filed Mar. 7, 2025, which is incorporated herein in its entirety by reference.

### FIELD OF THE INVENTION

The invention relates generally to biosensors, and more particularly to bioresorbable tags for monitoring deep tissue homeostasis.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Disruption of homeostasis represents a core feature of disease pathogenesis. Monitoring homeostasis at relevant anatomical sites thus provides essential physiological and pathological information crucial to early diagnosis before the onset of externally observable symptoms. Recent work demonstrates the potential for use of bioelectronic devices in continuous, noninvasive detection of changes in parameters related to homeostasis, including blood pressure and flow, temperature, extracellular fluid pH, blood glucose, tissue oximetry and cerebral interstitial fluid. Measurement modalities rely on optical, thermal, or radiofrequency signals of physiological processes. Attenuation in biological tissues limits the use of these mechanisms to shallow depths, sometimes up to the centimeter scale, but insufficient for assessments of deep tissues. Approaches to monitoring homeostasis in deep tissues such as computed tomography (CT), X-ray imaging or biopsies are often high-cost and invasive, and not compatible with continuous detection. In this context, advanced methods on ultrasound imaging are of growing interest, due to their ease of use, wide availability, absence of radiation exposure, and capacity to probe to significant depths (10 cm or more) at high spatial resolution (few hundred microns). Recent advances in functional ultrasound imaging indicate possibilities for monitoring physiological and microbiological dynamics in complex environments.

Therefore, a heretofore unaddressed need exists in the art to address the aforementioned deficiencies and inadequacies.

### SUMMARY OF THE INVENTION

In one aspect, the invention relates to a tag for real-time monitoring of homeostasis in tissue of a subject. The tag comprises a soft, shape-adaptive matrix member configured to be attached onto the tissue, wherein the soft, shape-adaptive matrix member undergoes dimensional changes upon interactions with its surrounding biofluid environment by amounts that depend on a local parameter of interest around the tissue; and a plurality of markers embedded in the soft, shape-adaptive matrix member, such that the dimensional changes of the soft, shape-adaptive matrix member lead to changes in spacings between the markers by magnitudes that are quantitatively determinable by imaging.

In one embodiment, the soft, shape-adaptive matrix member and the markers are configured to undergo dissolution reactions and hydrolytic chain scissions in biofluids, thereby leading to conversion into benign products over well-defined timescales.

In one embodiment, the plurality of markers is symmetrically distributed in the soft, shape-adaptive matrix member.

In one embodiment, the plurality of markers is circularly distributed in the soft, shape-adaptive matrix member, which allows ultrasonic, CT and/or MRI visualization in a manner that is independent of the orientation of the markers.

In one embodiment, each marker comprises an ultrasonic indicator that is detectable in ultrasound, CT and/or MRI images.

In one embodiment, each indicator comprises a disc of a bioresorbable metal air cavity selected to maximize the acoustic impedance mismatch with the surrounding biofluid environment to ensure strong contrast in ultrasound images.

In one embodiment, the bioresorbable metal comprises Mg, Zn, Fe, W, or Mo.

In one embodiment, the ultrasound tag is a bioresorbable, shape-adaptive, ultrasound, CT and/or MRI readable materials structure (BioSUM).

In one embodiment, the soft, shape-adaptive matrix member is formed of a stimulus-responsive material that is fine-tuned to respond to changes in the surrounding biofluid environment of the soft, shape-adaptive matrix member.

In one embodiment, the changes in the surrounding biofluid environment are induced by homeostatic perturbations in the tissue.

In one embodiment, the homeostatic perturbations are of gastric leaks, small intestinal leaks, and/or pancreatic leaks.

In one embodiment, the stimulus-responsive material comprises a pH-responsive hydrogel, designed to maximize its response in relevant pH ranges while maintaining stable mechanical properties.

In one embodiment, the pH-responsive hydrogel is designed to respond to different pH changes by using the protonation behavior of tertiary amine and carboxylic acid, such that a change in pH values of the surrounding biofluid environment results in swelling of the soft, shape-adaptive matrix member, which in turn causes the markers in the soft, shape-adaptive matrix member to predictably spread apart, which can be continuously monitored through ultrasound images.

In one embodiment, the pH-responsive hydrogel is designed to responsive to pH values as low as 1.0 in an acidic condition for monitoring of the gastric leaks.

In one embodiment, the pH-responsive hydrogel comprises poly[2-(dimethylamino)ethyl methacrylate-co-2-(diisopropylamino)ethyl methacrylate][p(DMAEMA-DPAEMA)] with a weight ratio of DMAEMA:DPAEMA being 7:3, with polyethylene glycol diacrylate (PEGDA) as a crosslinker.

In one embodiment, the pH-responsive hydrogel is designed to be responsive to pH values of about 6.8 in a condition that is only slightly lower than normal physiological levels of about 7.4, for monitoring the small intestinal leaks.

In one embodiment, the pH-responsive hydrogel comprises p(DMAEMA-DPAEMA) with a weight ratio of DMAEMA:DPAEMA being 9:1, with PEGDA as a crosslinker.

In one embodiment, the pH-responsive hydrogel is designed to be responsive to pH values larger than 7.4 in an alkaline condition for monitoring of the pancreatic leaks.

In one embodiment, the pH-responsive hydrogel comprises poly(acrylic acid-butyl acrylate) [p(AAc-BA)] with PEGDA as a crosslinker.

In one embodiment, the tag is usable for real-time monitoring of pH changes associated with anastomotic leakage following GI surgeries, pleural effusion, ascites and complex infections.

In one embodiment, the stimulus-responsive material is designed to maximize its response to other forms of homeostatic dysregulation including brain hemorrhage, wherein the stimulus-responsive material incorporates a hemostatic agent binding with red blood cells and absorption of blood, which results in its expansion as the basis for ultrasound detection.

In one embodiment, the stimulus-responsive material comprises a thermos-responsive material that incorporates polyethylene glycol grafted into a chitosan hydrogel, thereby enabling sensing of changes in temperature.

In one embodiment, the stimulus-responsive material is designed to maximize its response to inflammation or flare-ups in bowels of people with irritable bowel syndrome, and/or sepsis.

In one embodiment, the stimulus-responsive material is designed to maximize its response to biomolecules including drug metabolites or antibody accumulation, and/or pathogens including bacteria or viruses.

In one embodiment, the ultrasound tag is configured to real-time monitor the body's response to a drug, thereby eliminating the need for multiple invasive blood draws, and/or track the onset of infection while in resource-limited settings.

In one embodiment, the soft, shape-adaptive matrix member has an anchor feature for suturing the soft, shape-adaptive matrix member to the tissue.

In one embodiment, the soft, shape-adaptive matrix member is attached onto the tissue by bioresorbable adhesives.

In one embodiment, the tag is implantable to the tissue.

In one embodiment, the tag is injectable to the tissue.

In one embodiment, the tag is bioresorbable and/or biodegradable.

In one embodiment, the tag is usable for post-surgical monitoring of patient status throughout a recovery period, after which resorption into the body via hydrolysis and natural metabolic reactions naturally eliminates the tag, thereby eliminating the need for surgical extraction procedures subsequent to a desired timeframe for operation.

In one embodiment, the tag is configured to incorporate ultrasound image processing software in the workflow, with automated feature detection and/or artificial intelligence, to allow the patient to detect postoperative complications and return to the hospital if needed.

In another aspect, the invention relates to a method of real-time monitoring of homeostasis in tissue of a subject. The method comprises attaching at least one tag onto at least one surface of the tissue, wherein the at least one tag comprises a soft, shape-adaptive matrix member that undergoes dimensional changes upon interactions with its surrounding biofluid environment by amounts that depend on a local parameter of interest around the tissue; and a plurality of markers embedded in the soft, shape-adaptive matrix member such that the dimensional changes of the soft, shape-adaptive matrix member lead to changes in spacings between the markers; acquiring images of the plurality of markers; and determining the changes in the spacings between the markers from the acquired images, thereby determining changes in the surrounding biofluid environment and real-time monitoring of homeostasis in the tissue.

In one embodiment, the plurality of markers is symmetrically distributed in the soft, shape-adaptive matrix member.

In one embodiment, the plurality of markers is circularly distributed in the soft, shape-adaptive matrix member, which allows ultrasonic, CT and/or MRI visualization in a manner that is independent of the orientation of the markers.

In one embodiment, each marker comprises an ultrasonic indicator that is detectable in ultrasound, CT and/or MRI images.

In one embodiment, each indicator comprises a disc of a bioresorbable metal air cavity selected to maximize the acoustic impedance mismatch with the surrounding biofluid environment to ensure strong contrast in ultrasound images.

In one embodiment, the bioresorbable metal comprises Mg, Zn, Fe, W, or Mo.

In one embodiment, the tag is of a bioresorbable, shape-adaptive, ultrasound, CT and/or MRI readable materials structure (BioSUM).

In one embodiment, the soft, shape-adaptive matrix member is formed of a stimulus-responsive material that is fine-tuned to respond to changes in the surrounding biofluid environment of the soft, shape-adaptive matrix member.

In one embodiment, the changes in the surrounding biofluid environment are induced by homeostatic perturbations in the tissue.

In one embodiment, the homeostatic perturbations are of gastric leaks, small intestinal leaks, and/or pancreatic leaks.

In one embodiment, the stimulus-responsive material comprises a pH-responsive hydrogel, designed to maximize its response in relevant pH ranges while maintaining stable mechanical properties.

In one embodiment, the pH-responsive hydrogel is designed to respond to different pH changes by using the protonation behavior of tertiary amine and carboxylic acid, such that a change in pH values of the surrounding biofluid environment results in swelling of the soft, shape-adaptive matrix member, which in turn causes the markers in the soft, shape-adaptive matrix member to predictably spread apart, which can be continuously monitored through ultrasound images.

In one embodiment, the pH-responsive hydrogel is designed to responsive to pH values as low as 1.0 in an acidic condition for monitoring of the gastric leaks.

In one embodiment, the pH-responsive hydrogel is synthesized by performing a chain extension reaction of a mixture of 2-(dimethylamino)ethyl methacrylate monomer (DMAEMA) and 2-(diisopropylamino)ethyl methacrylate monomer (DPAEMA) with a weight ration of DMAEMA:DPAEMA being about 7:3; adding about 2 wt % 2-hydroxy-2-methylpropiophenone photoinitiator, followed by UV irradiation to yield a monomer mixture; subsequently adding about 3.85 wt % poly(ethylene glycol) diacrylate (PEGDA) and about 0.5 wt % 2,2-dimethoxy-2-phenylacetophenone photoinitiator (DMPA) to the monomer mixture to form a hydrogel precursor; and UV exposing to the hydrogel precursor under a nitrogen atmosphere to yield a crosslinked hydrogel network.

In one embodiment, the pH-responsive hydrogel is designed to responsive to pH values of about 6.8 in a condition that is only slightly lower than normal physiological levels of about 7.4, for monitoring of the small intestinal leaks.

In one embodiment, the pH-responsive hydrogel is synthesized by performing a chain extension reaction of a mixture of DMAEMA and DPAEMA with a weight ration of DMAEMA:DPAEMA being about 9:1; adding about 2 wt % 2-hydroxy-2-methylpropiophenone photoinitiator, followed by UV irradiation to yield a monomer mixture; subsequently adding about 3.85 wt % PEGDA and about 0.5 wt % DMPA to the monomer mixture to form a hydrogel precursor; and UV exposing to the hydrogel precursor under a nitrogen atmosphere to yield a crosslinked hydrogel network.

In one embodiment, the pH-responsive hydrogel is designed to responsive to pH values larger than 7.4 in an alkaline condition for monitoring of the pancreatic leaks.

In one embodiment, the pH-responsive hydrogel is synthesized by performing a chain extension reaction of butyl acrylate monomer (BA); adding about 0.5 wt % 2-hydroxy-2-methylpropiophenone photoinitiator, followed by UV irradiation to yield a BA oligomer; subsequently adding about 21.8 wt % of a pH-responsive monomer acrylic acid (AAc), about 1 wt % PEGDA and about 0.5 wt % DMPA photoinitiator to a BA oligomer to form a hydrogel precursor to the hydrogel; and UV exposing to the hydrogel precursor under a nitrogen atmosphere to yield a crosslinked hydrogel network.

In one embodiment, the method is usable for real-time monitoring of pH changes associated with anastomotic leakage following GI surgeries, pleural effusion, ascites and complex infections.

In one embodiment, the stimulus-responsive material is designed to maximize its response to other forms of homeostatic dysregulation including brain hemorrhage, wherein the stimulus-responsive material incorporates a hemostatic agent binding with red blood cells and absorption of blood, which results in its expansion as the basis for ultrasound detection.

In one embodiment, the stimulus-responsive material comprises a thermos-responsive material that incorporates polyethylene glycol grafted into a chitosan hydrogel, thereby enabling sensing of changes in temperature.

In one embodiment, the stimulus-responsive material is designed to maximize its response to inflammation or flare-ups in bowels of people with irritable bowel syndrome, and/or sepsis.

In one embodiment, the stimulus-responsive material is designed to maximize its response to biomolecules including drug metabolites or antibody accumulation, and/or pathogens including bacteria or viruses.

In one embodiment, the method is usable for real-time monitoring of the body's response to a drug, thereby eliminating the need for multiple invasive blood draws, and/or track the onset of infection while in resource-limited settings.

In one embodiment, said attaching at least one tag comprises one of suturing the soft, shape-adaptive matrix member to the tissue; adhering the soft, shape-adaptive matrix member to the tissue; and injecting the tag, thus the soft, shape-adaptive matrix member to the tissue.

In one embodiment, said adhering the soft, shape-adaptive matrix member to the tissue is formed with a photo-triggered liquid-solid transformation of the bioresorbable adhesive that enables the formation of bonding without applying external forces, thereby facilitating the attachment procedure, and minimizing the potential damage to the tissues.

In one embodiment, said adhering the soft, shape-adaptive matrix member to the tissue comprises coating the tissue surface with a primer layer including chitosan, sulfated N-hydroxysuccinimide (Sulfo-NHS) and (1-ethyl-3-(3-dimethylaminopropyl) carbodiimide (EDC), followed by applying a two-component bioresorbable adhesive onto the tag and the tissue surface, wherein the two-component adhesive is a viscous liquid solution of a photocurable covalent network of polyethylene glycol-lactide acid diacrylate (PEG-LA-DA) and an ionic network of sodium alginate; and exposing them to UV light to complete the attachment of the tag onto the tissue surface with robust chemical bonding.

In one embodiment, said attaching at least one tag comprises attaching multiple tags at strategic anatomical locations relative to a surgical site provide the basis for spatio-temporal monitoring of homeostasis.

These and other aspects of the invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 shows bioresorbable, shape-adaptive ultrasound-readable materials structures (BioSUM) for real-time monitoring of homeostasis in deep tissues. Panel A: Schematic illustration of a device, which includes a sparse collection of metal discs embedded in a thin hydrogel. Panel B: BioSUM enables ultrasonic monitoring of homeostasis in deep tissues. Panel C: Ultrasonic signals of BioSUM indicate homeostatic perturbations. Panel D: Subsequent bioresorption of BioSUM eliminates the need for surgical extraction. Panel E: Miniaturized designs allow for implantation by laparoscopic surgery. Images of three BioSUMs of different dimensions (12 mm, 7 mm, 3 mm in diameter) on the finger (left), wrapped around an O.D.=⅛-inch plastic tube (top right), and in the bent state (bottom right). Panel F: Schematic illustration of BioSUM for detecting post-surgical leakage on the stomach, small intestine, and pancreas. Panel G: pH-responsive ranges for BioSUM1, BioSUM2 and BioSUM3, and the corresponding pH values of representative digestive fluids. Panel H: Images of the accelerated dissolution of BioSUM2 in PBS (pH 7.4) solution at 95° C.

FIG. 2 shows pH-responsive behaviors of shape-adaptive materials structures. Panel A: Chemical structures of the monomers, oligomers and crosslinker in the pH-responsive hydrogels in BioSUM. Panel B: Schematic illustration of the mechanism for pH-responsive hydrogels in BioSUM1 and BioSUM2. Panel C: Schematic illustration of the mechanism for the pH-responsive hydrogel in BioSUM3. Panel D: Time-dependent response of BioSUM1 when immersed in a solution with pH 4.5. Panel E: FEA modeling results of the case shown in (D). Panels F, I, L: Ultrasonic measurement of time-dependent responses of BioSUM to immersion in solutions with different pH values. ΔL/L₀ (%) denotes the swelling ratio. Panel F: Response of BioSUM1. Panel I: Response of BioSUM2. Panel L: Response of BioSUM3. Panels G, J, M: Response of BioSUM to immersion in solutions with different pH values at the equilibrium state. Panel G: Response of BioSUM1. Panel J: Response of BioSUM2. Panel M: Response of BioSUM3. Panels H, K, N: Optical measurement of sensitivity of BioSUM to the introduction of different amounts of simulated GI fluids, including the response time for reaching 10% ΔL/L₀ and ΔL/L₀% at 30 min. Panel H: Response of BioSUM1 to simulated gastric fluid. Panel K: Response of BioSUM2 to simulated small intestinal fluid. Panel N: Response of BioSUM3 to simulated pancreatic fluid. Panel O: Schematic illustration of the setup for spatio-temporal mapping of pH with BioSUM1. Panel P: Spatio-temporal characteristics of BioSUM1 in response to the introduction of simulated gastric fluid (SGF) at one end of the system via optical methods. BioSUMs are fully swollen in PBS (pH 7.4) for 24 h before the measurements presented here.

FIG. 3 shows sensing by ultrasound imaging in deep tissues. Panel A: Schematic illustration of the cross-sectional position of acoustic waves relative to BioSUM in different cases, and the corresponding signals in the ultrasound B-mode image. Panel B: The symmetric design enables ultrasonic visualization independent of the orientation of the transducer. Panel C: Schematic illustration of BioSUM in a tilted case and the corresponding signals in the ultrasound B-mode image. Panel D: Ultrasound B-mode images of a BioSUM on the stomach in a rat model demonstrating the cross-sectional position of acoustic waves in different cases, correlated to the schematic illustration in panel A. Panel E: Ultrasound B-mode image of a BioSUM in a tilted case on the stomach in a rat model, corresponding to the schematic illustration in panel C. Panel E: Experimental and numerical simulation results for the measurement accuracy of BioSUM at different depths. Δx/x₀ is the deviation of measured length divided by the actual length. Panel G: Experimental and numerical simulation results for the signal-to-noise ratio (SNR) at different depths. Inset images are schematic illustrations of the image contrast in decibels.

FIG. 4 shows longitudinal monitoring of pH homeostasis of the rat gastrointestinal system. Panel A: Schematic illustration of the implantation and sensing procedure for BioSUM1 on the stomach of a rat. Panel B: Images showing the expansion of BioSUM1 2 h after creating a single gastrotomy. Panel C: Ultrasound images of BioSUM1 on the stomach over a 14-day stabilization period without induced gastric leakage. The depth of the array of Zn discs changes between day 0 and day 1 due to postoperative relocation of organs. Panel D: Longitudinal ultrasound images of BioSUM1 on the stomach following acute gastrotomy. Panel E: Summary data showing the swelling ratios of BioSUM1 during the stabilization period and following acute gastrotomy. n=3 biologically independent animals. RM One-Way ANOVA, Holm-Sidak's multiple comparison test vs Day 14 (0 min), 10 min, p=0.0208, 20 min, p=0.0208, 30 min, p=0.0198, 45 min, p=0.0207, 60 min, p=0.0208, 90 min, p=0.0092, 120 min, p=0.0207. Inset ultrasound images representing day 1, day 14 and 120 min are extracted from (Panel C) and (Panel D). Panel F: Same as (Panel E), but for BioSUM2 and enterotomy. RM One-Way ANOVA, p=0.0009, Holm-Sidak's multiple comparison test vs Day 14 (0 min), 10 min, p=0.0254, 20 min, p=0.0254, 30 min, p=0.0141, 45 min, p=0.0254, 60 min, p=0.0254, 90 min, p=0.0005, 120 min, p=0.02541. Inset ultrasound images representing day 1, day 14 and 120 min are extracted from FIG. 30. Panel G: Same as (Panel E), but for BioSUM3 and pancreatic leakage. RM One-Way ANOVA, p=0.0021, Holm-Sidak's multiple comparison test vs Day 14 (0 min), 10 min, p=0.0231, 20 min, p=0.0359, 30 min, p=0.0359, 45 min, p=0.0359, 60 min, p=0.0348, 90 min, p=0.0325, 120 min, p=0.0089. Dots represent individual animals. Line represents B-spline. Error bars represent ±SD. *p<0.05, **p<0.01, ***p<0.001. Inset ultrasound images representing day 1, day 14 and 120 min are extracted from FIG. 31. BioSUMs are fully swollen in PBS (pH 7.4) for 24 h before implantation, in all cases.

FIG. 5 shows deep-tissue detection of gastrointestinal leaks in porcine models. Panel A: Schematic illustration of the experimental procedures. Panel B: Ultrasound images of BioSUMs on the small intestine (left, BioSUM2), stomach (middle, BioSUM1), and pancreas (right, BioSUM3) in a pig. Panel C: Images showing the expansion of BioSUM3 after pancreatic leakage. Panel D: Longitudinal ultrasound images of BioSUM1 after acute gastrotomy. Panel E: Summary data showing the swelling ratio of BioSUM1 with or without acute gastrotomy. n=3 independent samples/group. Two-way ANOVA, Holm-Sidak's multiple comparison with gastrotomy T=0 min, gastrotomy, 30 min, p=0.0055, 45 min, p=0.0024, 60 min, p=0.0015, 90 min, p=0.0019, 120 min, p=0.0024; Sham 30 min, p=0.8568. Panel F: Same as (D), but for BioSUM2 and enterotomy. Panel G: Same as (E), but for BioSUM2 and enterotomy. n=3 independent samples/group. Two-way ANOVA, Holm-Sidak's multiple comparison with enterotomy T=0 min, enterotomy, 30 min, p=0.0351, 45 min, p=0.0120, 60 min, p=0.0120, 90 min, p=0.0028, 120 min, p=0.0049; Sham 30 min, p=0.9970. Panel H: Same as (D), but for BioSUM3 and pancreatic leakage. Dots represent individual samples. Line represents B-spline. Error bars represent ±SD. *p<0.05, **p<0.01. Panel I: Same as (E), but for BioSUM3 and pancreatic leakage. n=3 independent samples/group. Two-way ANOVA, Holm-Sidak's multiple comparison with pancreatic leakage T=0 min, pancreatic leakage, 30 min, p=0.0045, 45 min, p=0.0045, 60 min, p=0.0045, 90 min, p=0.0026, 120 min, p=0.0045; Sham 30 min, p=0.9988. BioSUMs are fully swollen in PBS (pH 7.4) for 24 h before all the implantation procedures.

FIG. 6 shows fixation and integration of a BioSUM onto the surfaces of soft living tissues using a bioresorbable adhesive. Panel A: Images of a BioSUM implanted in a rat model to demonstrate the adhesion between the device and the tissue. Panel B: Images of a BioSUM on explanted tissue to indicate the strength of the adhesion.

FIG. 7 shows images of BioSUM that highlight the miniaturized and flexible designs, to facilitate implantation during laparoscopic surgeries. Panel A: One BioSUM rolled in a plastic tube with I.D.=3 mm, O.D.=5 mm. Panel B: Image of a BioSUM after rolled in the plastic tube.

FIG. 8 shows images of the accelerated dissolution of BioSUM1 in PBS (pH 7.4) solution at 95° C.

FIG. 9 shows images of the accelerated dissolution of BioSUM3 in PBS (pH 7.4) solution at 95° C.

FIG. 10 shows characterization of the kinetics of biodegradation through measurements of the weight of hydrogels without Zn disks as a function of time of immersion in PBS (pH=7.4) at 37° C., 75° C., and 95° C. Panel A: BioSUM1. Panel B: BioSUM2. Panel C: BioSUM3.

FIG. 11 shows pH-responsive behaviors and ultrasonic detection of BioSUMs after degradation in physiological conditions at 37° C. in PBS. Panels A-C: pH-responses at the equilibrium state after degradation for 15 days and 30 days. Panel A: BioSUM1. Panel B: BioSUM2. Panel C: BioSUM3. Panels D-F: Images of devices in PBS (pH 7.4) solution at 37° C. after 30 days. Panel D: BioSUM1. Panel E: BioSUM2. Panel F: BioSUM3. Panels G-I: Ultrasound B-mode images of devices between two pork phantoms at 2 cm depth after degradation for 30 days. Panel G: BioSUM1. Panel H: BioSUM2. Panel I: BioSUM3. Panel J: Comparison of the signal quality before and after degradation in PBS at 37° C. for 30 days. Δx/x₀ is the deviation of measured length divided by the actual length.

FIG. 12 shows simplified schematic illustration of the bioresorption of hydrogel materials used in BioSUMs.

FIG. 13 shows materials designs for BioSUM1 and BioSUM2. Equilibrium swelling ratios of hydrogel materials with different DMAEMA and DPAEMA ratios at relevant pH values. ΔL/L₀ (%) denotes the swelling ratio.

FIG. 14 shows stress-strain curves of pH-responsive BioSUM materials after fully swollen in simulated fluids. Panel A: BioSUM1 after swollen in SGF (pH=1.2). Panel B: BioSUM2 after swollen in SIF (pH=6.8). (C) BioSUM3 after swollen in SPJ (pH=8.2).

FIG. 15 shows materials designs for BioSUM3. Equilibrium swelling ratios of hydrogel materials with different AAc ratios and degrees of crosslinking (PEGDA ratios) compared to BioSUM3 at relevant pH values. ΔL/L₀ (%) denotes the swelling ratio.

FIG. 16 shows numerical simulation results for the effects of different parameters on SNR values at 10 cm depth at a frequency of 5 MHz. Panel A: Effect of Zn thickness. Panel B: Effect of metal type. Panel C: Effect of the diameter of Zn disks. Panel D: Effect of the number of Zn disks (including Zn disk at the center).

FIG. 17 shows diffusivity (D) as a function of swelling ratio (J). A second-order continuous step function of swelling ratio J models the diffusivity D. Four parameters, Dₗ, Dₕ, J_{mid}, and J_{range}, determine the function.

FIG. 18 shows the geometry configuration of the model. The front and bottom planes are symmetric planes.

FIG. 19 shows the fitted X values at different pH values for three types of hydrogels. Panel A: BioSUM1. Panel B: BioSUM2. Panel C: BioSUM3.

FIG. 20 shows FEA results for thickness-dependent swelling ratios of BioSUM1 at different pH values after 10 min. ΔL/L₀% denotes the percentage change in length.

FIG. 21 shows designs of BioSUMs. Panel A: Design for the large animal model. Panel B: Design for the small animal model.

FIG. 22 shows comparison between the pH response of BioSUM1 and the hydrogel for BioSUM1 without the Zn disks, evaluated at the equilibrium states.

FIG. 23 shows pH-responsive behavior of BioSUMs designed for a rat model. Panels A, C, E: Time-resolved responses of BioSUM to different pH values. ΔL/L₀ (%) denotes the swelling ratio in length. Panel A: Response of BioSUM1. Panel C: Response of BioSUM2. Panel E: Response of BioSUM3. Panels B, D, F: Response of BioSUM to different pH values at the equilibrium state. Panel B: Response of BioSUM1. Panel D: Response of BioSUM2. Panel F: Response of BioSUM3.

FIG. 24 shows stress-strain curves of pH-responsive hydrogels in BioSUMs after fully swollen in PBS (pH 7.4) solution. Panel A: BioSUM1. Panel B: BioSUM2. Panel C: BioSUM3.

FIG. 25 shows wear rates of the hydrogel materials used in BioSUMs. Panel A: Wear rates of the hydrogel materials in BioSUM1, BioSUM2, and BioSUM3. Error bars represent ±SD. Panel B: Images of BioSUMs before the measurement of wear rates. Panel C: Images of BioSUMs after 100 cycles of the wear test.

FIG. 26 shows the performance of BioSUM1 after fully swollen in simulated gastric fluid in a rat model. Panel A: Images of a fully swollen BioSUM1 at the initial stage and after 2 h. Panel B: Ultrasound images of a fully swollen BioSUM1 at the initial stage and after 2 h. Panel C: Summary data showing ultrasonic measurements of swelling ratios at the initial stage and after 2 h. n=3 biologically independent animals.

FIG. 27 shows spatio-temporal characteristics of BioSUM2 in response to the introduction of simulated intestinal fluid (SIF) at one end of the system.

FIG. 28 shows spatio-temporal characteristics of BioSUM3 in response to the introduction of simulated pancreatic fluid (SPJ) at one end of the system.

FIG. 29 shows a theoretical model in one-dimension that illustrates the reflection of acoustic waves at the metal/tissue interface. Panel A: Schematic illustration of the model. Panel B: The relationship between the amplitude of the reflected wave vs. the thickness of the metal.

FIG. 30 shows BioSUM with a manually introduced tear implanted in a rat model. Panel A: Image of a BioSUM1 with a tear. The dashed box indicates the cross-sectional position of acoustic waves used to produce the image in panel B. Panel B: Ultrasound image of a BioSUM1 with a tear, where the acoustic waves intersect a diameter of the device without the tear. Panel C: Image of the same BioSUM1 shown in panel A. The dashed box indicates the cross-sectional position of acoustic waves used to produce the image in panel D. Panel D: Ultrasound image of a BioSUM1 with a tear, where the acoustic waves intersect a diameter of the device with the tear.

FIG. 31 shows a folded BioSUM1 in a rat model. Panel A: Image of a folded BioSUM1 on the stomach. The dashed box indicates the cross-sectional position of acoustic waves in panel B. Panel B: Ultrasound image of a folded BioSUM1 indicating the signal of five bright segments, different from the normal pattern.

FIG. 32 shows alternative design of BioSUMs with two suture rings on opposite sides. Panel A: Alternative design with two suture rings for enhanced mechanical fixation to tissues in the dynamic GI tract. Panels B, C: Images of a BioSUM1 on an elastomeric substrate with two loose sutures through the suture sites. Panel B: Image of a BioSUM1 before swelling. Panel C: Image of a BioSUM1 after free swelling to approximately 40% without constraint due to the existence of a gap between the suture and the ring.

FIG. 33 shows the expansion of a BioSUM in a curvilinear tissue environment. Panel A: Images of BioSUM1 on a curvilinear surface in a rat model before and after expansion. Panel B: Images of BioSUM1 on a flat substrate before and after expansion on this curvilinear surface, indicating isotropic expansion under biological conditions. Panel C: Summary results from panels A and B showing the deviation of distances between each pair of Zn disks (Zn disk on the perimeter and Zn disk in the center). Δx/\tilde{x} (%) denotes the percentage of deviation between the measured distance and the average distance. Dots represent the distance between each pair of Zn disks. Error bars represent ±SD. Panel D: FEA modeling results showing the deviation of distances between each pair of Zn at different swelling ratios. ΔL/L₀ (%) denotes the swelling ratio. Dots represent the distance between each pair of Zn disks. Error bars represent ±SD. Panel E: FEA modeling results demonstrating the deviation of distances between each pair of Zn disks at different circular angles. The swelling ratio of BioSUM is 50%. Panel F: FEA modeling results for a BioSUM on a column with a diameter of 2.5 cm (left) and in a flattened state (right) indicating isotropic expansion. The swelling ratio of the BioSUM is 50%.

FIG. 34 shows schematic illustrations of ultrasonic measurements from a bent device. Panel A: The actual length x₀. Panel B: The ultrasonic measurement length x. Panel C: Schematic illustration with auxiliary lines for calculating the deviation between x₀ and x.

FIG. 35 shows numerical simulation results for the effects of different parameters on the measurement accuracy at 10 cm depth and a frequency of 5 MHz. Panel A: Effect of the diameter of Zn disks. Panel B: Effect of the number of Zn disks (including Zn disk at the center). Δx/x₀ is the deviation of the measured length divided by the actual length.

FIG. 36 shows numerical simulation results for ultrasound B-mode images at different depths for a frequency of 5 MHz.

FIG. 37 shows schematic illustration of the simulation model for ultrasound. Panel A: The transducer. Panel B: The tissue with an embedded device.

FIG. 38 shows schematic illustration of the experimental setup for ultrasonic characterization of measurement accuracy and signal-to-noise ratio (SNR) using pork phantoms.

FIG. 39 shows examples of length measurements and characterization of SNR values by MATLAB coding using the ultrasound image from pancreatic leakage at 60 min in a porcine model. Panel A: Measurement of length. Panel B: Characterization of SNR.

FIG. 40 shows ultraminiaturized design guided by numerical simulation for operation at depths of up to 10 cm. Panel A: Numerical simulation results for the measurement accuracy and SNR as a function of the diameter of Zn disks. A hydrogel with a diameter of 3 mm is used. For hydrogels with diameters of less than 3 mm, measurement accuracy and SNR values cannot meet the requirements of Δx/x₀<0.1 and SNR>18 dB, respectively. The frequency of the ultrasonic waves is 8 MHz. Panel B: Device design. Panel C: Image of a device on the finger. Panel D: Image of a slightly bent device in a plastic tube with I.D.= 1/16 inch, O.D.=⅛ inch.

FIG. 41 shows ultrasound B-mode image of BioSUM1 on the stomach in a rat model. The labels identify the representative organs in the GI tract.

FIG. 42 shows detection of BioSUM using two different ultrasound imaging systems. Panel A: Ultrasound B-mode images of BioSUM1 on the stomach in a rat model using a Canon Xario 200 system with 18L7 transducer (top) and a GE Logiq P9 system with ML6-15 transducer (bottom). Panel B: Comparison of the signal quality from the two ultrasound imaging systems. Δx/x₀ is the deviation of measured length divided by the actual length.

FIG. 43 shows longitudinal monitoring of pH homeostasis in a rat model after a properly performed GI surgery. Panel A: Images showing the procedures associated with a properly performed GI surgery (gastrotomy) and postoperative monitoring via a BioSUM1. The wound closure procedure after the gastrotomy involves a simple interrupted suture on the first layer, followed by a continuous lambert pattern on the second layer to invert the tissue. Panel B: Ultrasound images of the BioSUM1 on the stomach over a 14-day period after the GI surgery. The depth of the array of Zn disks changes between day 0 and day 1 due to postoperative relocation of the organs. Panel C: Longitudinal ultrasound images of the BioSUM1 on the stomach following a separate gastrotomy. Panel D: Summary data showing the swelling ratios of the BioSUM1 over the 14-day period after the GI surgery and following a separate gastrotomy on day 14. n=4 biologically independent animals. RM One-Way ANOVA, p<0.0001, Holm-Sidak's multiple comparison test vs Day 14 (0 min), 10 min, p=0.0255, 20 min, p=0.0069, 30 min, p=0.0016, 45 min, p=0.0051, 60 min, p=0.0033, 90 min, p=0.0051, 120 min, p=0.0047. Inset ultrasound images representing day 1, day 14 and 120 min are extracted from (B) and (C).

FIG. 44 shows experimental and numerical simulations of the effects of mechanical deformation of organs adjacent to the device on the length measurement. Panel A: Image of BioSUM1 on the stomach in a rat showing the interstitial fluid that exists between the devices and the tissue. Panel B: Experimental and FEA modeling results showing the effect of mechanical deformation on the length measurement. x₀ denotes the measured distance between each pair of Zn disks (Zn disk on the perimeter and Zn disk in the center) before mechanical deformation. x denotes the measured distance after mechanical deformation. Dots represent individual measurement. Error bars represent ±SD. Panel C: Images of BioSUM1 on an elastomeric substrate with the presence of PBS solution at the interface. A pair of fiducials on the elastomeric substrate indicates a stretching ratio of ~60%. Panel D: Images from FEA modeling before and after the mechanical deformation of the substrate indicating minimal effect on the device. U denotes displacement.

FIG. 45 shows effect of protein (bovine serum albumin, BSA) on pH-responsive behaviors of BioSUMs. Panel A: Summary data showing minimal fluctuations in dimensions after immersion in PBS solution with 1 wt % BSA (pH=7.4) at 37° C. for 2 weeks. BioSUMs were fully swollen in PBS for 24 h before the test. Panels B-D: pH-responses of BioSUMs at the equilibrium state after immersion in PBS solution with 1 wt % BSA at 37° C. for 2 weeks. pH buffer solutions with 1 wt % BSA were used for characterizing pH-responses. Panel B: BioSUM1. Panel C: BioSUM2. Panel D: BioSUM3. ΔL/L₀ (%) denotes the swelling ratio. Measurements only involve narrow pH ranges to avoid the denaturation of the BSA.

FIG. 46 shows the performance of BioSUM1 for detection of gastric leakage in a rat model without the 14-day stabilization period. Panel A: Swelling ratios of BioSUM1 following gastrotomy. n=6 biologically independent animals. RM One-Way ANOVA, p<0.0001, Sidak's multiple comparison test vs 0 min, 10 min, p=0.0152, 20 min, p=0.0006, 30 min, p=0.0001, 45 min, p=0.0002, 60 min, p=0.0008, 90 min, p=0.0008, 120 min, p=0.0006. Dots represent individual animals. Line represents B-spline. Error bars represent ±SD. *p<0.05, **p<0.01, ***p<0.001, ****p<0.0001. Panel B: Images after gastrotomy.

FIG. 47 shows the performance of BioSUM1 during gastric leakage and after the termination of leakage in a rat model. Panel A: Summary data showing the swelling ratios of BioSUM1 following gastrotomy for 2 h and the stabilized swelling ratios 6 h after termination of leakage. n=6 biologically independent animals. RM One-Way ANOVA, p<0.0001, Sidak's multiple comparison test vs 0 h, 2 h, p=0.0002, 8 h, p=0.0001. Dots represent individual animals. Line represents B-spline. Error bars represent ±SD. *p<0.05, **p<0.01, ***p<0.001, ****p<0.0001. Panel B: Images of BioSUM1 at the initial stage, 2 h following gastrotomy, and 6 h after termination of leakage. Panel C: Ultrasound images of BioSUM1 at the initial stage, 2 h following gastrotomy, and 6 h after termination of leakage.

FIG. 48 shows ultrasound images of BioSUM2 before and immediately after small intestine leakage in a rat model. Panel A: Ultrasound images of BioSUM2 on the small intestine over a 14-day period prior to small intestinal leakage. The depth of the device changed between day 0 and day 1 due to postoperative relocation of organs. Panel B: Images following enterotomy.

FIG. 49 shows ultrasound images of BioSUM3 before and immediately after pancreatic leakage in a rat model. Panel A: Ultrasound images of BioSUM3 on the pancreas over a 14-day period prior to pancreatic leakage. The depth of the device changed between day 0 and day 1 due to postoperative relocation of organs. Panel B: Images following pancreatic leakage.

FIG. 50 shows the performance of BioSUM2 for detection of small intestinal leakage in a rat model without the 14-day stabilization period. Panel A: Swelling ratios of BioSUM2 following enterotomy. n=6 biologically independent animals. RM One-Way ANOVA, p<0.0001, Sidak's multiple comparison test vs 0 min, 10 min, p=0.0205, 20 min, p=0.0003, 30 min, p=0.0002, 45 min, p=0.0006, 60 min, p=0.0007, 90 min, p=0.0008, 120 min, p=0.0006. Dots represent individual animals. Line represents B-spline. Error bars represent ±SD. *p<0.05, **p<0.01, ***p<0.001, ****p<0.0001. Panel B: Images after enterotomy.

FIG. 51 shows the performance of BioSUM3 for detection of pancreatic leakage in a rat model without the 14-day stabilization period. Panel A: Swelling ratios of BioSUM3 following pancreatic leakage. n=6 biologically independent animals. RM One-Way ANOVA, p<0.0001, Sidak's multiple comparison test vs 0 min, 10 min, p=0.0016, 20 min, p=0.0007, 30 min, p=0.0017, 45 min, p=0.0025, 60 min, p=0.0008, 90 min, p=0.0024, 120 min, p=0.0018. Dots represent individual animals. Line represents B-spline. Error bars represent ±SD. *p<0.05, **p<0.01, ***p<0.001, ****p<0.0001. Panel B: Images after leakage.

FIG. 52 shows monitoring small intestinal leakage via a BioSUM2 following a poorly performed intestinal anastomosis. Panel A: Images showing the procedures of a poorly performed intestinal anastomosis that introduces leakage. The surgical procedure starts with an enterotomy by disconnecting the intestine in the middle, followed by holding the two halves of the intestine with a flat hemostat. A poorly performed simple continuous suture of the joint induces small intestinal leakage. Panel B: Swelling ratios of the BioSUM2 following the poorly performed intestinal anastomosis. n=3 biologically independent animals. RM One-Way ANOVA, p=0.0072, Holm-Sidak's multiple comparison test vs 0 min, 10 min, p=0.0302, 20 min, p=0.0302, 30 min, p=0.0191, 45 min, p=0.0191, 60 min, p=0.0191, 90 min, p=0.0191, 120 min, p=0.0126. Dots represent individual animals. Line represents B-spline. Error bars represent ±SD. *p<0.05, **p<0.01, ***p<0.001, ****p<0.0001. Panel C: Ultrasound images of the BioSUM2 on the small intestine after intestinal anastomosis.

FIG. 53 shows real-time pH dynamics of the local environment associated with leaks in a rat model after a 14-day stabilization period. Panel A: Gastric leakage. Panel B: Small intestinal leakage. Panel C: Pancreatic leakage.

FIG. 54 shows histology images of GI organs of sham controls and rats with BioSUMs implanted for 2 and 4 weeks.

FIG. 55 shows FEA modeling results for the swelling performance of a BioSUM in a fibrous cap. Panel A: Image of a BioSUM3 in a fibrous cap. The fibrous cap has a Young's modulus of 20 kPa and a thickness of 25 μm. S denotes von Mises stress. Panel B: Summary results showing minimal effect of the fibrous cap on the swelling ratio. ΔL/L₀ (%) denotes the swelling ratio without the fibrous cap. L denotes the measured distance between Zn disks under the free swelling condition. I denotes the measured distance during the swelling procedure with the presence of a fibrous cap.

FIG. 56 shows in vivo biocompatibility and toxicity studies. Panel A: Analysis of blood chemistry. n=4 biologically independent animals. GLU, glucose (mg/dL); TRIG, triglycerides (mg/dL); ALT alanine aminotransferase (U/L); AST, aspartate transaminase (U/L); ALP, alkaline phosphatase (U/L); CHOL, cholesterol (mg/dL); Cl, chloride (mmol/L); Na, sodium (mmol/L); Na/K, sodium/potassium ratio; TCO2, bicarbonate (mmol/L). Two-way ANOVA, Holm-Sidak's multiple comparison test with Sham group, p>0.05 for all experimental groups except for GLU 4-week, AST 4-week and ALP. Panel B: Analysis of blood chemistry. n=4 biologically independent animals. PHOS, phosphorus (mg/dL); Cal, calcium (mg/dL); K, potassium (mmol/L); Urea, blood urea nitrogen (mg/dL); ALB, albumin (g/dL); GLOB, globulin (g/dL); A/G, albumin/globulin ratio; CREA, creatinine (mg/dL); TBIL, total bilirubin (mg/dL); TP, total protein (g/dL). Two-way ANOVA, Holm-Sidak's multiple comparison test with sham group, p>0.05 for all experimental groups. Panel C: Element analysis of the biodistribution of Zn. n=4 biologically independent animals. Two-way ANOVA, Holm-Sidak's multiple comparison test with the sham group, p>0.05 for all experimental groups.

FIG. 57 shows images of L929 live/dead cells after 7 days of culture. Arrows indicate the positions of devices.

FIG. 58 shows in vitro cytocompatibility of BioSUMs using L929 cells (mouse fibroblast cell line) after 1 day, 4 days, and 7 days of culture. Cytotoxic concentration of DMSO served as a positive control, while PEGDA was used as a negative control. n=3 individual samples. Two-way ANOVA, Holm-Sidak's multiple comparison with cell in complete medium. *p<0.05, **p<0.01, ***p<0.001, ****p<0.0001. ns represents not statistically significant.

FIG. 59 shows ultrasound sensor with air cavities. Panel A: Design of the sensor with air cavities. Panel B: Optical image of a sensor with air cavities. Panel C: Ultrasound B-mode image of a sensor in a pork phantom of 1 cm depth. Panel D: Swelling performances of the ultrasound sensor with air cavities following gastric leakage in a rat model. Panel E: Ultrasound B-mode images after gastric leakage. Panel F: Swelling performances of the ultrasound sensor with air cavities following small intestinal leakage in a rat model. Panel G: Ultrasound B-mode images after small intestinal leakage.

FIG. 60 shows MRI sensors. Panel A: Design of the sensor for MRI imaging. Panels B-G: Optical images of sensors with different types and concentrations of iron oxide particles. Panels H-M: MRI T2 weighted images of sensors. Panels B, H: 1 wt % Fe₃O₄ nanoparticles as MRI contrast agent. Panels C, I: 10 wt % Fe₃O₄ nanoparticles as MRI contrast agent. Panels D, J: 1 wt % Fe₃O₄ microparticles as MRI contrast agent. Panels E, K: 10 wt % Fe₃O₄ microparticles as MRI contrast agent. Panels F, L: 1 wt % Fe₂O₃ microparticles as MRI contrast agent. Panels G, M: 10 wt % Fe₂O₃ microparticles as MRI contrast agent. Panel N: MRI T2 weighted image of a sensor with 10 wt % Fe₂O₃ microparticles implanted on the stomach in a rat.

FIG. 61 shows CT sensors. Panel A: Design of the sensor for CT imaging. Panels B-F: Optical images of sensors with different types and thickness of metal discs. Panels G-K: CT images of sensors. Panels B, G: 10 μm thick Zn as CT contrast agent. Panels C, H: 12.5 μm thick Mo as CT contrast agent. Panels D, I: 25 μm thick Zn as CT contrast agent. Panels E, J: 30 μm thick Mg as CT contrast agent. Panels F, K: 50 μm thick W as CT contrast agent. Panel L: CT image of sensors with different types of metal discs implanted in a mouse.

FIG. 62 shows temperature-responsive hydrogel sensors. Panel A: The design of temperature-responsive hydrogel sensor. Panel B: Chemical structures of the monomers, oligomers in the temperature-responsive hydrogels. Panel C: Schematic illustration of the mechanism for temperature-responsive hydrogels. Panel Panel D: Temperature-dependent swelling behavior of sensor. ΔL/L₀% denotes the swelling ratio. Panel E: Time-dependent responses of temperature-responsive hydrogels, to immersion in solutions with different temperature values. ΔL/L₀% denotes the swelling ratio.

FIG. 63 shows MRI readable sensors. Panel A: Design of the sensor for MRI imaging. Panels B-F: MRI T2 weighted images of sensors with different types of metal disc in PBS. Panel B: 25 μm thick 20 wt % Fe₂O₃ nanoparticles dispersed inside the PEGDA film as MRI contrast agent. Panel C: 25 μm thick Zn disc as MRI contrast agent. Panel D: 25 μm thick Mo disc as MRI contrast agent. Panel E: 25 μm thick W disc as MRI contrast agent. Panel F: 25 μm thick Mg disc as MRI contrast agent. Panel G: MRI T2 weighted image of a sensor with 20 wt % Fe₂O₃ microparticles dispersed inside the PEGDA film after acute gastrotomy in a rat.

FIG. 64 shows CT readable sensors. Panel A: Design of the pH-responsive sensor for CT imaging. Panels B-F: CT images of sensors with different types of metal discs. Panel B: 25 μm thick 20 wt % Fe₂O₃ nanoparticles dispersed PEGDA film as CT contrast agent. Panel C: 25 μm thick Zn as CT contrast agent. Panel D: 25 μm thick Mo as CT contrast agent. Panel E: 25 μm thick W as CT contrast agent. Panel F: 25 μm thick Mg as CT contrast agent. Panel F: CT image of sensors with Zn discs after acute gastrotomy in a rat.

FIG. 65 shows materials for ultrasound hydrogel tags. Panel A: Adhesive hydrogel tag arrays embedded bioresorbable metals (Mo) are bonded to the transient support via hydrogen bonding. Panel B: Adhesive hydrogel with NHS functional groups is chemically bonded to amine groups of target tissue via amine-NHS reaction. Panel C: The transient matrix supports transfer of tag arrays to target organ tissue, by maintaining the structure of arrays. Panel D: Transient support matrix is quickly dissolved into biofluid within 3-5 min by biofluids. Biopolymers including hyaluronic acid and synthetic polymers including PVA which is fast dissolution rate in water/biofluids can be used for the transient support.

FIG. 66 shows after dissolution of transient support, displacements of tag arrays (Δl=l₁−l₀) measured to monitor mechanical deformations of the organ by ultrasound images.

FIG. 67 shows the ultrasound images before and after dissolution of the transient support.

FIG. 68 shows characterization of ultrasound hydrogel tags. Panel A: Tag arrays exhibit lower errors in measuring displacements Δl than the bulk hydrogel systems. This is because of elastic modulus mismatch between tissue and hydrogel. Panel B: Finite element simulation (FEM) results show that mechanical deformations of each hydrogel system on the bladder tissue (10 kPa). Hydrogel tag arrays follow strain of the stretched bladder tissue, enabling minimizing measurement errors. By contrast, bulk hydrogel (100 kPa and thickness of 100 m) shows no significant changes in displacements on the bladder tissue, due to the elastic mismatch. Panels C-D: Ultrasound images clearly show displacements changes of each array. Panel E: Tag arrays on the pork tissue show stable adhesion, enabling follow mechanical deformations of port tissue.

FIG. 69 shows the hydrogel tag arrays integration on tissues (here, bladder surface) without surgical stitches (Pabel A), and after transfer of a hydrogel tag array system, a transient support matrix is quickly dissolved by biofluids, remaining only tag arrays (Panel B).

FIG. 70 shows in vivo validation of ultrasound hydrogel tag arrays. Panels A-B: Tag arrays transferred on the bladder shows stable adhesion property without significant changes in displacements between tag arrays over 4 weeks in vivo. Panel C: Tag arrays designed symmetric structures with repeated angle of 60° enables local strain mapping with its specific angular directions (−60°, 0°, 60°) of tissues. Panel D: Local strain with different angular directions was measured at different volumes of infused saline into the bladder.

FIG. 71 shows experimental measurements of local strain showing its asymmetric expansion of bladder as a function of the volume of infused saline into the bladder. However, the measured total displacement in each direction matches the lateral expansion of the bladder. The hydrogel tag arrays enable non-invasive monitoring capabilities of mechanical organ deformation, providing spatial strain information of the tissue.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

The term “flexibility” or “bendability”, as used in the disclosure, refers to the ability of a material, structure, device or device component to be deformed into a curved or bent shape without undergoing a transformation that introduces significant strain, such as strain characterizing the failure point of a material, structure, device or device component. In an exemplary embodiment, a flexible material, structure, device or device component may be deformed into a curved shape without introducing strain larger than or equal to 5%, for some applications larger than or equal to 1%, and for yet other applications larger than or equal to 0.5% in strain-sensitive regions. A used herein, some, but not necessarily all, flexible structures are also stretchable. A variety of properties provide flexible structures (e.g., device components) of the invention, including materials properties such as a low modulus, bending stiffness and flexural rigidity; physical dimensions such as small average thickness (e.g., less than 100 microns, optionally less than 10 microns and optionally less than 1 micron) and device geometries such as thin film and open or mesh geometries.

The term “bending stiffness” refers to a mechanical property of a material, device or layer describing the resistance of the material, device or layer to an applied bending moment. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

In modern medicine, devices that can monitor biological changes in cells and organs are essential to understanding, diagnosing, and managing disease. However, many limitations exist in current monitoring devices, particularly in those that aim to detect changes deep within tissues. For example, high cost, invasiveness, and lack of real-time feedback needs to be overcome to enable earlier detection and treatment of disease.

The invention discloses an innovative approach to monitoring using an implant called a bioresorbable, shape-adaptive, ultrasound-readable materials structure (BioSUM). This device could allow at-home monitoring of deep-tissue changes after surgery.

BioSUM is an implantable device composed of small metal discs within a pH-responsive hydrogel. The device could allow recovery at home after surgery and rapid detection of postoperative complications. For example, when carrying out gastrointestinal (GI) anastomosis surgeries, BioSUM can be implanted. During recovery at home, the distance between the metal discs is measured by ultrasound. If a leak occurs, the hydrogel swells, so the metal discs are further apart. This early detection would prompt a return to the hospital before substantial organ damage arises.

BioSUM is a millimeter-scale monitoring device. It is simple in form but complex in function. Composed of small metal discs embedded within a pH-responsive hydrogel matrix, the device is implanted into the body with the intended purpose of monitoring homeostasis in deep tissues. The thin and flexible nature of BioSUM confers shape adaptivity, allowing it to be rolled into a tube and shunted through a trocar during laparoscopic surgery, sutured to tissue, or placed directly on a surface of interest using an adhesive. The metal discs serve as visual indicators that can be readily detected on ultrasound, and their symmetric circular distribution allows for identification regardless of how the device is oriented when implanted. Unlike many medical implants that require an additional procedure to remove the device when its purpose is fulfilled, the metal discs and hydrogel matrix of BioSUM are bioresorbable, eliminating the need to retrieve the device or any residuals of the device.

Gastrointestinal leaks can occur as a complication of anastomosis surgeries (which involve connecting tubular structures), resulting in fluid spreading through the peritoneal cavity and causing organ damage. When BioSUM senses a pH change, such as in the case of a gastrointestinal leak, the chemical composition of the hydrogel matrix allows the device to swell. Polymers making up BioSUM were fine-tuned to respond to different pH changes by using the protonation behavior of tertiary amine and carboxylic acid. This causes the metal discs in BioSUM to predictably spread apart, which can be continuously monitored through conventional ultrasound.

In one embodiment, BioSUM were surgically sutured on the gastrointestinal organs of rats and pigs for 14 days, demonstrating its stability. Then, a gastrointestinal leak was induced, and they could detect changes in the geometry of the metal discs within 10 mins in rats and 30 mins in pigs. The information gathered from ultrasound imaging reveals the presence and magnitude of the leak, and thus the device can be used in postsurgical monitoring. The surgeon could simply place BioSUM on the tissue during the wound-closure procedure and send the patient home for recovery with confidence. Handheld ultrasound devices are accessible to the public, enabling the patient to monitor the implanted BioSUM at home. By incorporating ultrasound image processing software in the workflow, perhaps with automated feature detection or artificial intelligence, the patient could easily detect postoperative complications and return to the hospital.

Although post-surgical monitoring is a practical application for its use, which indeed may be how the technology is initially implemented, BioSUM is an important platform technology. The hydrogel matrix that enables it to swell in response to pH change can be tuned for different ranges of pH. Thus, three versions of BioSUM were created, demonstrating the ability to operate in three different environments: One version operates and detects leaks within the pH range of the stomach, another detects leaks in that of the gut, and another detects leaks from the pancreas. And although it has only been shown to detect leaks, BioSUM seems well-suited for other monitoring scenarios, such as for inflammation or flare-ups in the bowels of people with irritable bowel syndrome, or for sepsis, provided that the technology is adapted for such purposes.

Suppose the hydrogel matrix is swapped for an entirely different stimulus-responsive polymer. In this way, the device could respond to other cues, such as biomolecules (for example, drug metabolites or antibody accumulation) or pathogens (for example, bacteria or viruses). As a biosensor, the device could monitor the body's response to a drug, eliminating the need for multiple invasive blood draws, or it could track the onset of infection while in resource-limited settings. Given the numerous possibilities for device applications, a deeper investigation into the need for continuous at-home health monitoring may identify the application with the most promising potential for accelerated translation.

Monitoring technologies that meet societal demands for precise, personalized, and convenient health care are on the rise. BioSUM, in its current iteration, introduces a platform technology that yields the potential to fit within a repertoire of emerging monitoring tools, such as capsule-based diagnostics and ophthalmic imaging techniques, that enhance the way that disease can be understood, diagnosed, and managed.

The novel feature of the invention utilizes the BioSUM to convert local physiological changes, such as pH shifts associated with gastrointestinal leakage, into quantitative geometric changes measurable by ultrasound, CT, or MRI. Unlike conventional implantable markers that merely provide passive imaging contrast, this system embeds symmetrically arranged bioresorbable metal discs within a stimulus-responsive hydrogel matrix. When exposed to specific biochemical environments, the hydrogel undergoes controlled swelling, thereby altering inter-marker spacing in a predictable and image-detectable manner. This mechanical-geometry encoding of biochemical information, readable at deep tissue depths using standard clinical imaging systems, is structurally and functionally distinct from prior optical, electronic, or telemetry-based biosensors.

Additionally, the device is fully bioresorbable, dissolving into benign products after a defined monitoring period, eliminating the need for surgical removal. The combination of tunable hydrogel chemistries for distinct physiological ranges (e.g., gastric, intestinal, pancreatic environments), optimized acoustic impedance marker design, orientation-independent geometry, and compatibility with existing ultrasound infrastructure establishes a novel platform that integrates materials science, imaging physics, and surgical deployment in a way not previously disclosed.

The inventive step lies in the non-obvious integration of stimulus-responsive polymer chemistry with geometric acoustic encoding for deep-tissue monitoring, without electronics, batteries, telemetry, or wired components. Instead of transmitting biochemical signals electronically, the invention leverages controlled hydrogel expansion to modulate spatial relationships between embedded imaging markers, allowing quantitative readout using conventional ultrasound systems. This approach departs from traditional biosensing paradigms and would not be suggested by prior art focused on electronic implants, passive fiducial markers, or standalone pH-responsive materials.

Further, the system demonstrates technical sophistication in optimizing marker size, thickness, spacing, and material composition to achieve reliable signal-to-noise ratios at depths up to approximately 10 cm, while maintaining miniaturization compatible with laparoscopic surgery. The integration of bioresorbable adhesives, symmetric circular marker configurations for orientation-independent measurement, and validated in vivo performance across multiple animal models reflects a coordinated, multidisciplinary design that would not arise from routine modification of existing technologies.

The invention discloses specific monomer ratios, such as p(DMAEMA-DPAEMA) at 7:3 or 9:1, to create “tunable” sensors that respond exclusively to the specific pH signatures of gastric, intestinal, or pancreatic leaks. Furthermore, the integration of a two-component bioresorbable adhesive (utilizing PEG-LA-DA and sodium alginate) allows the tag to be chemically bonded to delicate wet tissue via UV light, ensuring the sensor remains in place during the critical postoperative recovery window before naturally dissolving.

The primary advantage of this technology is the elimination of secondary “extraction” surgeries, as the device is fully absorbed by the body once its monitoring task is complete. Clinically, it provides a means for the early detection of life-threatening anastomotic leaks before physical symptoms appear, potentially reducing hospital stay durations and healthcare costs. Additionally, the orientation-independent design of the marker arrays ensures that measurements remain accurate regardless of how the medical professional holds the ultrasound probe, making the system robust and user-friendly for routine clinical or even at-home monitoring.

The invention enables continuous, real-time monitoring of deep tissue homeostasis using widely available ultrasound equipment, allowing early detection of complications such as postoperative leaks before systemic symptoms develop. Because the device is fully bioresorbable, it avoids the need for secondary surgical retrieval procedures, reducing patient risk and healthcare costs. Its passive operation eliminates reliance on implanted electronics, thereby enhancing safety, simplifying design, and reducing long-term failure risks.

Moreover, the platform is adaptable: by modifying the stimulus-responsive matrix, it can potentially monitor temperature, hemorrhage, inflammation, drug metabolism, infection, or other biomarkers. The system integrates seamlessly into existing surgical workflows, supports at-home postoperative monitoring, and is compatible with automated image processing or AI-assisted analysis. Collectively, these features offer a scalable, cost-effective, and clinically practical solution for personalized deep tissue monitoring.

Without intent to limit the scope of the invention, exemplary embodiments of the invention are given below.

In one aspect, the invention relates to a tag for real-time monitoring of homeostasis in tissue of a subject. The ultrasound tag comprises a soft, shape-adaptive matrix member configured to be attached onto the tissue, wherein the soft, shape-adaptive matrix member undergoes dimensional changes upon interactions with its surrounding biofluid environment by amounts that depend on a local parameter of interest around the tissue; and a plurality of markers embedded in the soft, shape-adaptive matrix member, such that the dimensional changes of the soft, shape-adaptive matrix member lead to changes in spacings between the markers by magnitudes that are quantitatively determinable by imaging.

In some embodiments, the soft, shape-adaptive matrix member and the markers are configured to undergo dissolution reactions and hydrolytic chain scissions in biofluids, thereby leading to conversion into benign products over well-defined timescales.

In some embodiments, the plurality of markers is symmetrically distributed in the soft, shape-adaptive matrix member.

In some embodiments, the plurality of markers is circularly distributed in the soft, shape-adaptive matrix member, which allows ultrasonic, CT and/or MRI visualization in a manner that is independent of the orientation of the markers.

In some embodiments, each marker comprises an indicator that is detectable in ultrasound, CT and/or MRI images.

In some embodiments, each indicator comprises a disc of a bioresorbable metal air cavity selected to maximize the acoustic impedance mismatch with the surrounding biofluid environment to ensure strong contrast in ultrasound images.

In some embodiments, the bioresorbable metal comprises Mg, Zn, Fe, W, or Mo.

In some embodiments, the ultrasound tag is a bioresorbable, shape-adaptive, ultrasound, CT and/or MRI readable materials structure (BioSUM).

In some embodiments, the soft, shape-adaptive matrix member is formed of a stimulus-responsive material that is fine-tuned to respond to changes in the surrounding biofluid environment of the soft, shape-adaptive matrix member.

In some embodiments, the changes in the surrounding biofluid environment are induced by homeostatic perturbations in the tissue.

In some embodiments, the homeostatic perturbations are of gastric leaks, small intestinal leaks, and/or pancreatic leaks.

In some embodiments, the stimulus-responsive material comprises a pH-responsive hydrogel, designed to maximize its response in relevant pH ranges while maintaining stable mechanical properties.

In some embodiments, the pH-responsive hydrogel is designed to respond to different pH changes by using the protonation behavior of tertiary amine and carboxylic acid, such that a change in pH values of the surrounding biofluid environment results in swelling of the soft, shape-adaptive matrix member, which in turn causes the markers in the soft, shape-adaptive matrix member to predictably spread apart, which can be continuously monitored through ultrasound images.

In some embodiments, the pH-responsive hydrogel is designed to responsive to pH values as low as 1.0 in an acidic condition for monitoring of the gastric leaks.

In some embodiments, the pH-responsive hydrogel comprises poly[2-(dimethylamino)ethyl methacrylate-co-2-(diisopropylamino)ethyl methacrylate][p(DMAEMA-DPAEMA)] with a weight ratio of DMAEMA:DPAEMA being 7:3, with polyethylene glycol diacrylate (PEGDA) as a crosslinker.

In some embodiments, the pH-responsive hydrogel is designed to responsive to pH values of about 6.8 in a condition that is only slightly lower than normal physiological levels of about 7.4, for monitoring the small intestinal leaks.

In some embodiments, the pH-responsive hydrogel comprises p(DMAEMA-DPAEMA) with a weight ratio of DMAEMA:DPAEMA being 9:1, with PEGDA as a crosslinker.

In some embodiments, the pH-responsive hydrogel is designed to responsive to pH values larger than 7.4 in an alkaline condition for monitoring of the pancreatic leaks.

In some embodiments, the pH-responsive hydrogel comprises poly(acrylic acid-butyl acrylate) [p(AAc-BA)] with PEGDA as a crosslinker.

In some embodiments, the ultrasound tag is usable for real-time monitoring of pH changes associated with anastomotic leakage following GI surgeries, pleural effusion, ascites and complex infections.

In some embodiments, the stimulus-responsive material is designed to maximize its response to other forms of homeostatic dysregulation including brain hemorrhage, wherein the stimulus-responsive material incorporates a hemostatic agent binding with red blood cells and absorption of blood, which results in its expansion as the basis for ultrasound detection.

In some embodiments, the stimulus-responsive material comprises a thermos-responsive material that incorporates polyethylene glycol grafted into a chitosan hydrogel, thereby enabling sensing of changes in temperature.

In some embodiments, the stimulus-responsive material is designed to maximize its response to inflammation or flare-ups in bowels of people with irritable bowel syndrome, and/or sepsis.

In some embodiments, the stimulus-responsive material is designed to maximize its response to biomolecules including drug metabolites or antibody accumulation, and/or pathogens including bacteria or viruses.

In some embodiments, the ultrasound tag is configured to real-time monitor the body's response to a drug, thereby eliminating the need for multiple invasive blood draws, and/or track the onset of infection while in resource-limited settings.

In some embodiments, the soft, shape-adaptive matrix member has an anchor feature for suturing the soft, shape-adaptive matrix member to the tissue.

In some embodiments, the soft, shape-adaptive matrix member is attached onto the tissue by bioresorbable adhesives.

In some embodiments, the tag is implantable to the tissue.

In some embodiments, the tag is injectable to the tissue.

In some embodiments, the tag is bioresorbable and/or biodegradable.

In some embodiments, the tag is usable for post-surgical monitoring of patient status throughout a recovery period, after which resorption into the body via hydrolysis and natural metabolic reactions naturally eliminates the tag, thereby eliminating the need for surgical extraction procedures subsequent to a desired timeframe for operation.

In some embodiments, the tag is configured to incorporate ultrasound image processing software in the workflow, with automated feature detection and/or artificial intelligence, to allow the patient to detect postoperative complications and return to the hospital if needed.

In another aspect, the invention relates to a method of real-time monitoring of homeostasis in tissue of a subject. The method comprises attaching at least one tag onto at least one surface of the tissue, wherein the at least one tag comprises a soft, shape-adaptive matrix member that undergoes dimensional changes upon interactions with its surrounding biofluid environment by amounts that depend on a local parameter of interest around the tissue; and a plurality of markers embedded in the soft, shape-adaptive matrix member such that the dimensional changes of the soft, shape-adaptive matrix member lead to changes in spacings between the markers; acquiring images of the plurality of markers; and determining the changes in the spacings between the markers from the acquired images, thereby determining changes in the surrounding biofluid environment and real-time monitoring of homeostasis in the tissue.

In some embodiments, the plurality of markers is symmetrically distributed in the soft, shape-adaptive matrix member.

In some embodiments, the plurality of markers is circularly distributed in the soft, shape-adaptive matrix member, which allows ultrasonic, CT and/or MRI visualization in a manner that is independent of the orientation of the markers.

In some embodiments, each marker comprises a indicator that is detectable in ultrasound, CT and/or MRI images.

In some embodiments, each indicator comprises a disc of a bioresorbable metal air cavity selected to maximize the acoustic impedance mismatch with the surrounding biofluid environment to ensure strong contrast in ultrasound images.

In some embodiments, the bioresorbable metal comprises Mg, Zn, Fe, W, or Mo.

In some embodiments, the tag is of a bioresorbable, shape-adaptive, ultrasound, CT and/or MRI readable materials structure (BioSUM).

In some embodiments, the soft, shape-adaptive matrix member is formed of a stimulus-responsive material that is fine-tuned to respond to changes in the surrounding biofluid environment of the soft, shape-adaptive matrix member.

In some embodiments, the changes in the surrounding biofluid environment are induced by homeostatic perturbations in the tissue.

In some embodiments, the homeostatic perturbations are of gastric leaks, small intestinal leaks, and/or pancreatic leaks.

In some embodiments, the stimulus-responsive material comprises a pH-responsive hydrogel, designed to maximize its response in relevant pH ranges while maintaining stable mechanical properties.

In some embodiments, the pH-responsive hydrogel is designed to respond to different pH changes by using the protonation behavior of tertiary amine and carboxylic acid, such that a change in pH values of the surrounding biofluid environment results in swelling of the soft, shape-adaptive matrix member, which in turn causes the markers in the soft, shape-adaptive matrix member to predictably spread apart, which can be continuously monitored through ultrasound images.

In some embodiments, the pH-responsive hydrogel is designed to responsive to pH values as low as 1.0 in an acidic condition for monitoring of the gastric leaks.

In some embodiments, the pH-responsive hydrogel is synthesized by performing a chain extension reaction of a mixture of 2-(dimethylamino)ethyl methacrylate monomer (DMAEMA) and 2-(diisopropylamino)ethyl methacrylate monomer (DPAEMA) with a weight ration of DMAEMA:DPAEMA being about 7:3; adding about 2 wt % 2-hydroxy-2-methylpropiophenone photoinitiator, followed by UV irradiation to yield a monomer mixture; subsequently adding about 3.85 wt % poly(ethylene glycol) diacrylate (PEGDA) and about 0.5 wt % 2,2-dimethoxy-2-phenylacetophenone photoinitiator (DMPA) to the monomer mixture to form a hydrogel precursor; and UV exposing to the hydrogel precursor under a nitrogen atmosphere to yield a crosslinked hydrogel network.

In some embodiments, the pH-responsive hydrogel is designed to responsive to pH values of about 6.8 in a condition that is only slightly lower than normal physiological levels of about 7.4, for monitoring of the small intestinal leaks.

In some embodiments, the pH-responsive hydrogel is synthesized by performing a chain extension reaction of a mixture of DMAEMA and DPAEMA with a weight ration of DMAEMA: DPAEMA being about 9:1; adding about 2 wt % 2-hydroxy-2-methylpropiophenone photoinitiator, followed by UV irradiation to yield a monomer mixture; subsequently adding about 3.85 wt % PEGDA and about 0.5 wt % DMPA to the monomer mixture to form a hydrogel precursor; and UV exposing to the hydrogel precursor under a nitrogen atmosphere to yield a crosslinked hydrogel network.

In some embodiments, the pH-responsive hydrogel is designed to responsive to pH values larger than 7.4 in an alkaline condition for monitoring of the pancreatic leaks.

In some embodiments, the pH-responsive hydrogel is synthesized by performing a chain extension reaction of butyl acrylate monomer (BA); adding about 0.5 wt % 2-hydroxy-2-methylpropiophenone photoinitiator, followed by UV irradiation to yield a BA oligomer; subsequently adding about 21.8 wt % of a pH-responsive monomer acrylic acid (AAc), about 1 wt % PEGDA and about 0.5 wt % DMPA photoinitiator to a BA oligomer to form a hydrogel precursor to the hydrogel; and UV exposing to the hydrogel precursor under a nitrogen atmosphere to yield a crosslinked hydrogel network.

In some embodiments, the method is usable for real-time monitoring of pH changes associated with anastomotic leakage following GI surgeries, pleural effusion, ascites and complex infections.

In some embodiments, the stimulus-responsive material is designed to maximize its response to other forms of homeostatic dysregulation including brain hemorrhage, wherein the stimulus-responsive material incorporates a hemostatic agent binding with red blood cells and absorption of blood, which results in its expansion as the basis for ultrasound detection.

In some embodiments, the stimulus-responsive material comprises a thermos-responsive material that incorporates polyethylene glycol grafted into a chitosan hydrogel, thereby enabling sensing of changes in temperature.

In some embodiments, the stimulus-responsive material is designed to maximize its response to inflammation or flare-ups in bowels of people with irritable bowel syndrome, and/or sepsis.

In some embodiments, the stimulus-responsive material is designed to maximize its response to biomolecules including drug metabolites or antibody accumulation, and/or pathogens including bacteria or viruses.

In some embodiments, the method is usable for real-time monitoring of the body's response to a drug, thereby eliminating the need for multiple invasive blood draws, and/or track the onset of infection while in resource-limited settings.

In some embodiments, said attaching at least one tag comprises one of suturing the soft, shape-adaptive matrix member to the tissue; adhering the soft, shape-adaptive matrix member to the tissue; and injecting the tag, thus the soft, shape-adaptive matrix member to the tissue.

In some embodiments, said adhering the soft, shape-adaptive matrix member to the tissue is formed with a photo-triggered liquid-solid transformation of the bioresorbable adhesive that enables the formation of bonding without applying external forces, thereby facilitating the attachment procedure, and minimizing the potential damage to the tissues.

In some embodiments, said adhering the soft, shape-adaptive matrix member to the tissue comprises coating the tissue surface with a primer layer including chitosan, sulfated N-hydroxysuccinimide (Sulfo-NHS) and (1-ethyl-3-(3-dimethylaminopropyl) carbodiimide (EDC), followed by applying a two-component bioresorbable adhesive onto the tag and the tissue surface, wherein the two-component adhesive is a viscous liquid solution of a photocurable covalent network of polyethylene glycol-lactide acid diacrylate (PEG-LA-DA) and an ionic network of sodium alginate; and exposing them to UV light to complete the attachment of the tag onto the tissue surface with robust chemical bonding.

In some embodiments, said attaching at least one tag comprises attaching multiple tags at strategic anatomical locations relative to a surgical site provide the basis for spatio-temporal monitoring of homeostasis.

Without intent to limit the scope of the invention, exemplary instruments, apparatus, methods and their related results according to the embodiments of the invention are given below.

Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### Bioresorbable, Shape-Adaptive Structures for Ultrasonic Monitoring of Deep-Tissue Homeostasis

Monitoring homeostasis is an essential aspect in obtaining pathophysiological insights for treating patients. Accurate, timely assessments of homeostatic dysregulation in deep tissues typically require expensive imaging techniques or invasive biopsies.

In this example, we introduce a bioresorbable, shape-adaptive materials structure that enables real-time monitoring of deep-tissue homeostasis using conventional ultrasound instruments. Collections of small, bioresorbable metal discs distributed within thin, pH-responsive hydrogels, deployed by surgical implantation or syringe injection, allow ultrasound-based measurements of spatio-temporal changes in pH for early assessments of anastomotic leaks following gastrointestinal surgeries, where bioresorption after a recovery period eliminates the need for surgical extraction. Demonstrations in small and large animal models illustrate capabilities in monitoring leakage from the small intestine, the stomach, and the pancreas.

### Materials and Structural Designs

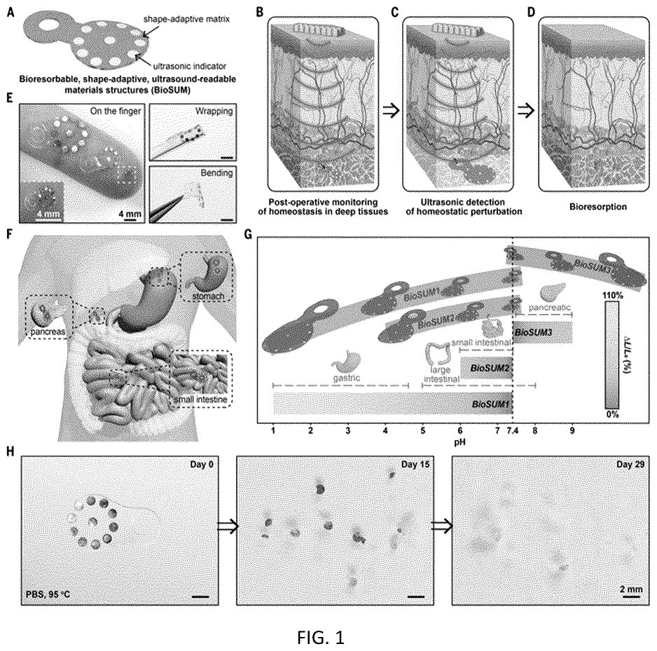

Millimeter-scale, bioresorbable materials structure are designed to enable real-time, spatio-temporal measurements of perturbations of chemical or physical homeostatic parameters in shallow or deep tissue locations by conventional ultrasound imaging, with specific clinically relevant uses in temporary patient monitoring (e.g., monitoring homeostatic dysregulation). These bioresorbable, thin, and flexible implants incorporate symmetrically distributed collections of structures that generate strong contrast in ultrasound images embedded in a soft, shape-adaptive matrix material (panel A of FIG. 1), referred to in the following as a bioresorbable, shape-adaptive, ultrasound-readable materials structure (BioSUM). A circular ring feature serves as an anchor for suturing the device to adjacent tissues. The matrix (green color in panel A of FIG. 1) undergoes well-defined dimensional changes upon interactions with the surrounding biofluid environment by amounts that depend on a local parameter of interest. These transformations change the spacings between the ultrasonic indicators by magnitudes that can be quantitatively determined by imaging. The indicators include small discs of bioresorbable metals (e.g., Mg, Zn, Fe, W, or Mo) selected to maximize the acoustic impedance mismatch with the surroundings to ensure strong contrast in ultrasound images. The envisioned clinical use case is in post-surgical monitoring of patient status throughout a recovery period, after which resorption into the body via hydrolysis and natural metabolic reactions naturally eliminates the devices, thereby bypassing the need for surgical extraction procedures (panels B-D of FIG. 1).

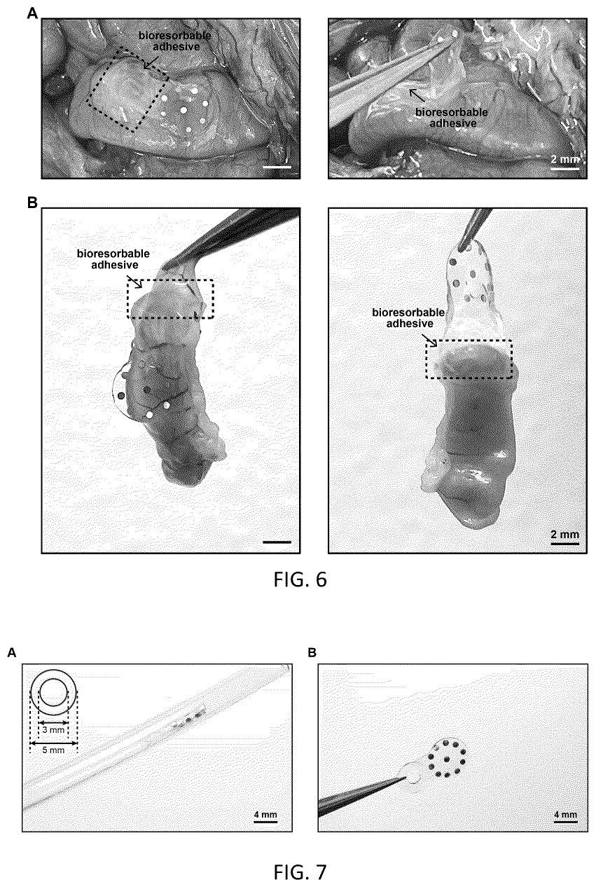

Homeostatic pH regulation is crucial for maintaining proper cellular and physiological functions. Perturbations may result from endogenous metabolic crises (e.g., acidosis or alkalosis) or exogenous events (e.g., surgical interventions or trauma). For example, leakage of gastrointestinal (GI) fluids secondary to anastomosis surgeries may cause significant alterations in local pH homeostasis, with the potential to propagate throughout the peritoneal cavity, leading to organ dysfunctions and failures. Accurate monitoring of the pH in tissues surrounding the anastomotic site thus offers an opportunity for early detection of leakage with high sensitivity and specificity, to allow for therapeutic interventions that can reduce the morbidity and mortality of postoperative complications in GI surgeries (ranging from 5% to 25% depending on the sites of anastomosis). The absence of standard clinical approaches for assessments and the vague patient symptoms in many cases pose additional challenges to early and accurate detection of anastomotic leakage. In this scheme, suturing one or more BioSUMs near the location of the anastomosis as the final step of the surgical procedure provides the basis for frequent monitoring via ultrasound imaging during a recovery period (FIG. 6 for an alternative approach to fixation with bioresorbable adhesives). The small dimensions of the devices, their thin geometries and soft, flexible mechanical properties provide an additional option in deployment, by syringe injection (panel E of FIG. 1, FIG. 7) and facilitate implantation during laparoscopic surgeries. Their bioresorbable construction eliminates the need for surgical extraction subsequent to a desired timeframe for operation. The examples presented here illustrate capabilities in monitoring gastric, small intestinal, and pancreatic leakage as three representative cases (panel F of FIG. 1).

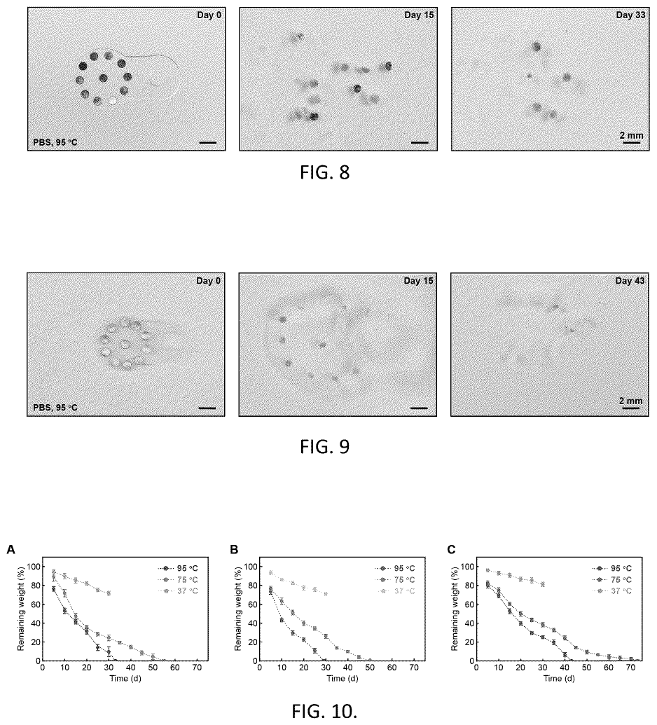

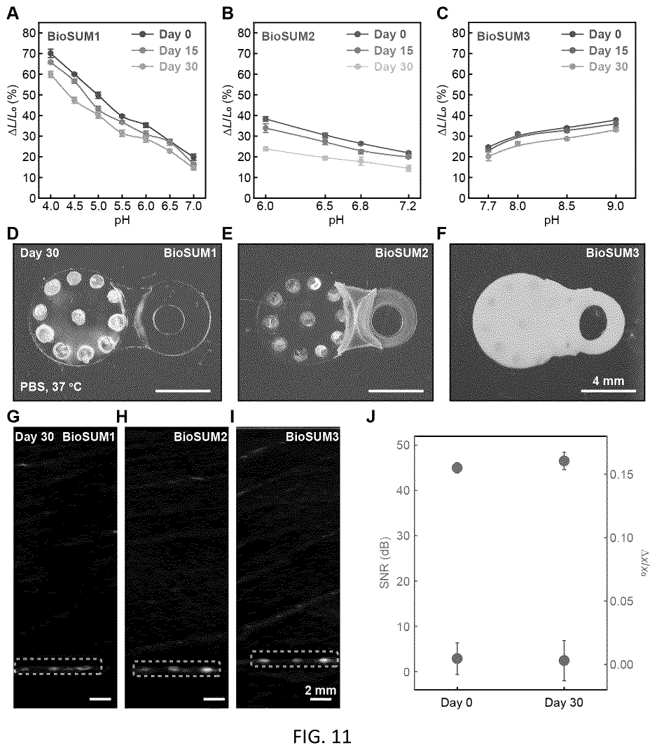

These use cases rely on hydrogels as pH-responsive matrix materials, tailored to operate across relevant ranges of pH values. Specifically, the gastric environment can present pH values as low as 1.0, thereby requiring hydrogels that swell in these acidic conditions while offering stable chemical and mechanical properties in these aggressive environments (BioSUM1). Monitoring for small intestinal leaks, by contrast, relies on hydrogels that operate at values of pH (~6.8) that are only slightly lower than normal physiological levels (~7.4) (BioSUM2). The pancreas involves alkaline conditions, thus demanding hydrogels that are responsive to pH values larger than 7.4 (BioSUM3). Panel G of FIG. 1 summarizes the ranges of pH relevant for these three classes of hydrogels and their corresponding organ interfaces. Each is also designed to be fully bioresorbable (panel H of FIG. 1, FIGS. 8-12). Choices of bioresorbable metals and hydrogel materials that undergo dissolution reactions and hydrolytic chain scissions in biofluids lead to conversion into benign products over well-defined timescales. Panel H of FIG. 1 illustrates the accelerated dissolution pathway of BioSUM2 in phosphate-buffered saline (PBS, pH=7.4) at 95° C. The device largely dissolves within 15 days, and the residual materials disappear entirely after 29 days. At body temperature, the latter timescale corresponds to 174, 160, and 241 days for BioSUM1, BioSUM2, and BioSUM3, respectively.

pH-Responsive Behavior

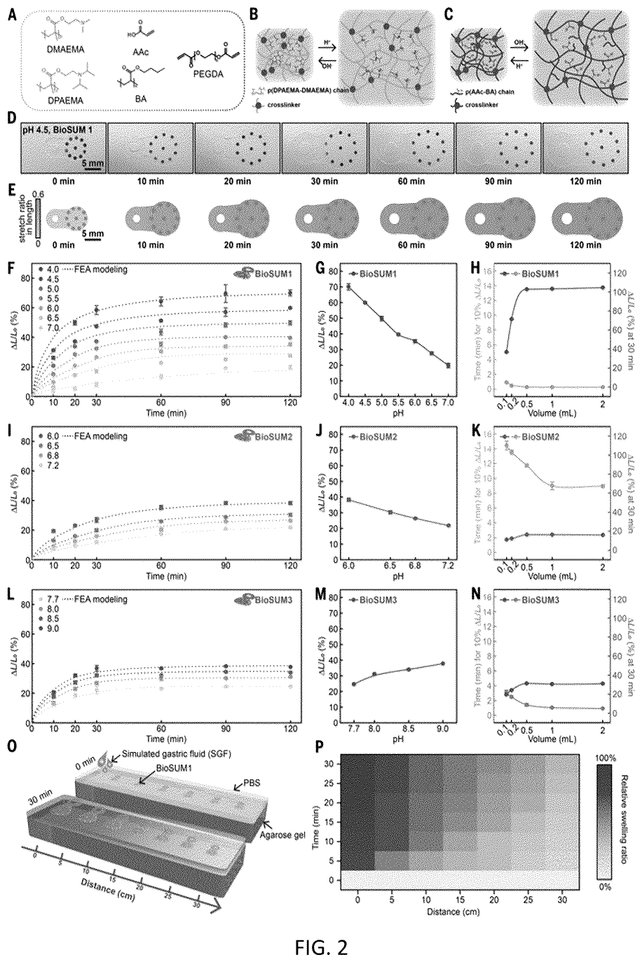

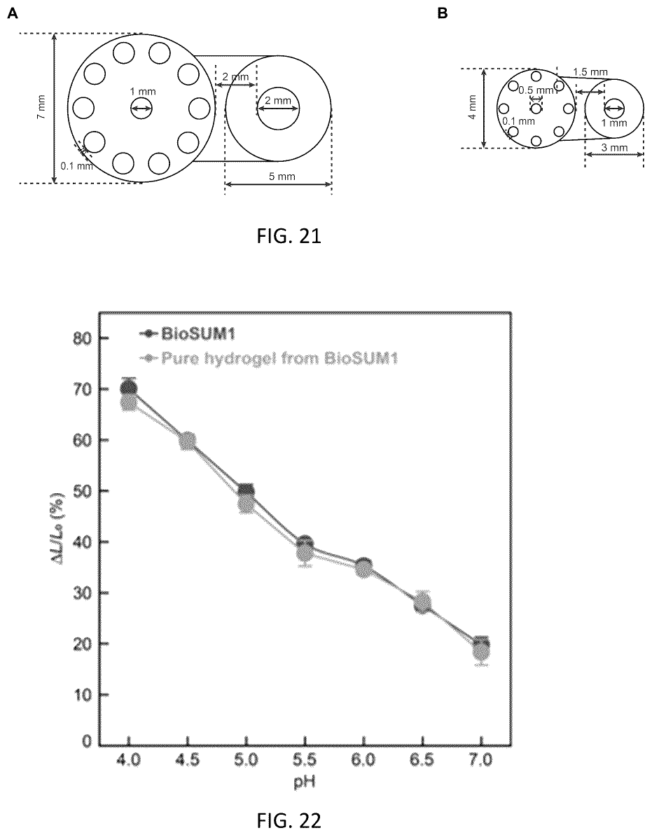

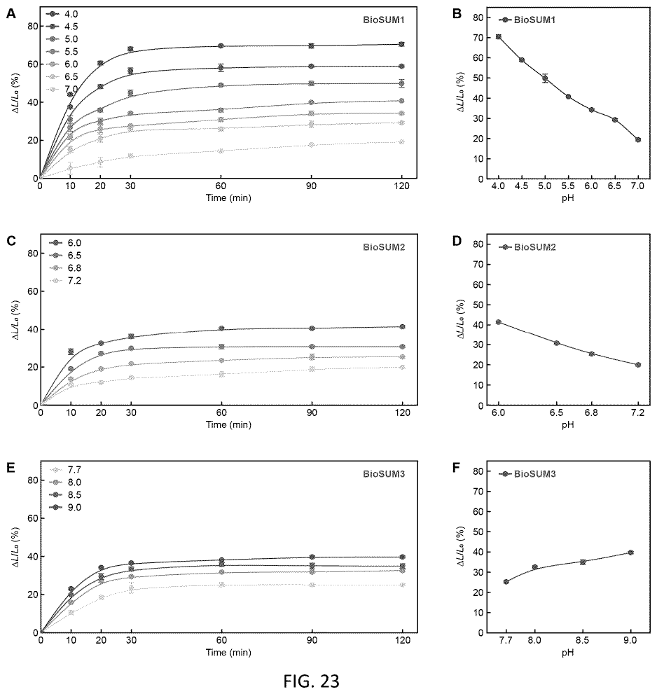

The pH responses of the hydrogels originate from their chemical compositions. Materials for gastric and small intestinal leaks involve poly [2-(dimethylamino)ethyl methacrylate-co-2-(diisopropylamino)ethyl methacrylate][p(DMAEMA-DPAEMA)] with polyethylene glycol diacrylate (PEGDA, Mₙ 250) as a crosslinker (panels A-B of FIG. 2). DMAEMA and DPAEMA contain tertiary amine moieties that undergo protonation and subsequent changes in osmotic pressure as the pH decreases. The hydrogel chemistry that responds in alkaline conditions relies on poly (acrylic acid-butyl acrylate) [p(AAc-BA)] with PEGDA as a crosslinker (panels A and C of FIG. 2). Elevation of pH results in protonation of the carboxyl moieties in AAc and a corresponding conformational change of the hydrogel network due to hydrophilicity. Specifically, the addition of hydrophobic BA hinders the protonation of carboxyl around the original pKa of AAc moiety (~4.25) and shifts the swelling transition to values larger than the physiological pH of 7.4 (see FIGS. 13-16, Tables 2-5 for details about materials designs). Finite element analysis (FEA) that includes the effects of coupled diffusion and deformation can capture the swelling kinetics in these polyelectrolyte hydrogels. Protonation of pH-responsive moieties enables the diffusion of buffer solutions into the crosslinked networks and successive expansion of elastomeric chains. The diffusivity (in the range of 10⁻⁸-10⁻¹² m²/s) and the thicknesses of the hydrogels largely determine the swelling kinetics. The swelling ratio correlates to the inverse square of thickness, i.e., ~τ/h², where τ is time, h is thickness. Physical entanglement and grafted side chains in the hydrogel networks contribute to considerable swelling ratios at the equilibrium state (see FIGS. 17-19 and Table 1 for more details about the swelling of hydrogels). FEA results predict the thickness-dependent swelling behavior of BioSUM1 for 10 min at different pH values, in terms of the percentage change in length (ΔL/L₀%) (FIG. 20). To balance the need for both fast response time and small dimensions, the circular parts of the devices reported here have thicknesses of 300 μm and diameters of 7 mm, with a symmetrically distributed collection of thin, circular discs of Zn (1 mm diameter and 25 μm thickness) located at the midpoint of the thickness of the hydrogel (panel A of FIG. 21). The discs induce no measurable mechanical constraints on the swelling of the hydrogel matrix (FIG. 22). Further miniaturized devices (4 mm diameter and 200 μm thickness), with expected enhancements in response times (panel B of FIG. 21 and FIG. 23) facilitate use in small animal models.

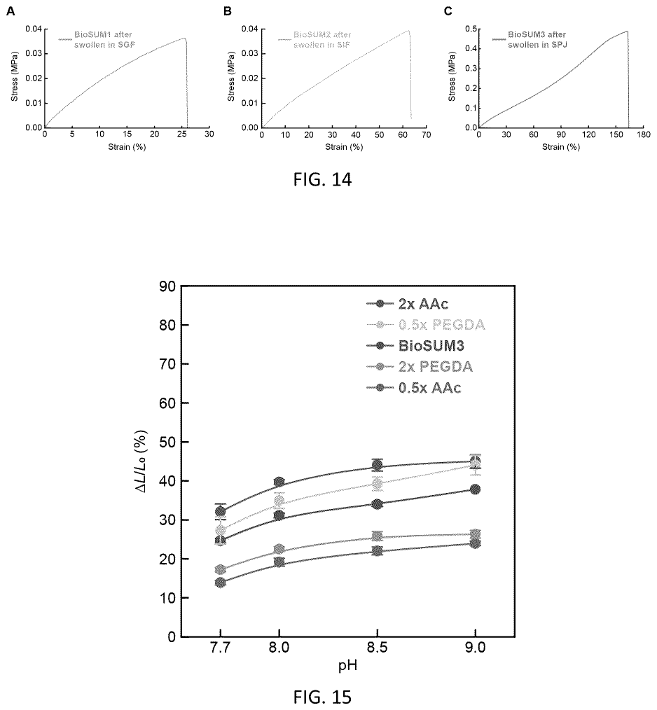

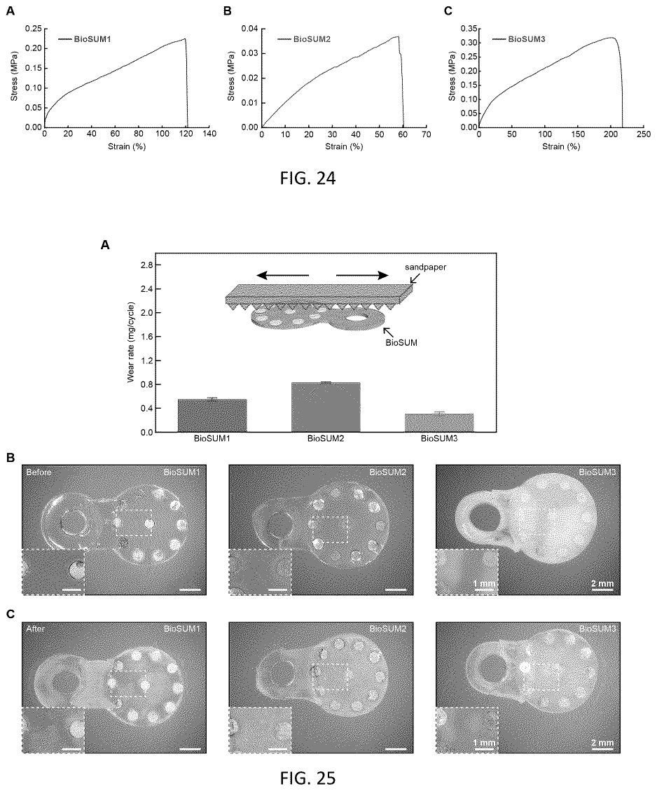

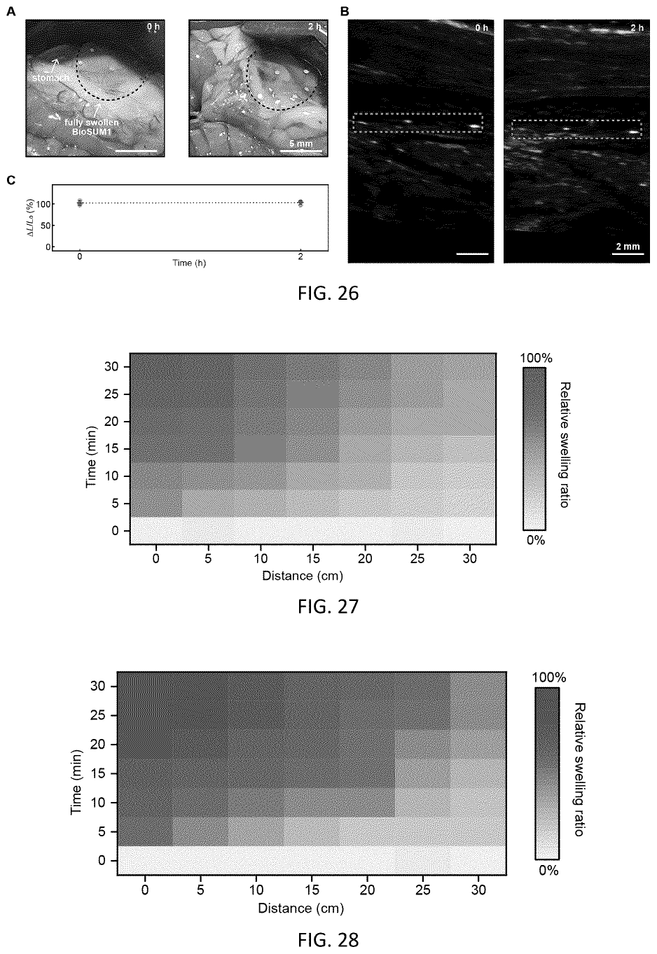

Panels D-E of FIG. 2 present experimental measurements and FEA results for the time-resolved swelling behavior of BioSUM1 in a citrate buffer solution at pH 4.5, respectively. Increasing the molar ratio of DPAEMA improves the mechanical robustness to ensure stable behavior in strongly acidic gastric fluids (FIGS. 14 and 24-26, Tables 2-4). This increase also, however, reduces the degree of swelling near physiological pH. Experimental measurements along with FEA modeling of the swelling ratios (ΔL/L₀%) for BioSUM1 and BioSUM2 appear in panels F-K of FIG. 2. As examples, BioSUM1 swells by 65% in pH 4.0 citrate buffer solution within 30 min. BioSUM2 swells by 12% in pH 7.2 in the same timeframe. When in direct contact with simulated gastric fluid (SGF, pH~1.2), BioSUM1 swells by 10% in less than 1 min and reaches over 35% in 30 min (panel H of FIG. 2). BioSUM2 can respond to 1 mL of simulated intestinal fluid (SIF, pH~6.8) applied directly onto the device, by swelling over 10% within 10 min and over 15% in 30 min (panel K of FIG. 2). Panels L and M of FIG. 2 present the response of BioSUM3 to alkaline conditions. BioSUM3 swells by 35% in pH 9 within 30 min (panel L of FIG. 2), to an equilibrium swelling ratio of ~40% (panel M of FIG. 2). BioSUM3 swells by 10% within 1 min after contact with 1 mL simulated pancreatic fluid (SPJ, pH~8.2), and it reaches 30% in 30 min (panel N of FIG. 2). FEA modeling accurately predicts the swelling kinetics for all cases (panels F, I, L of FIG. 2).

Multiple devices at strategic anatomical locations relative to a surgical site provide the basis for spatio-temporal monitoring of the convective spread of SGF (panels O and P of FIG. 2). Benchtop demonstrations involve the introduction of SGF on one end of a slab of agarose gel. Seven sensors distributed along a straight line in a thin layer of PBS solution on this gel allow measurements of the swelling ratios at corresponding locations, to a distance of 30 cm from the origin at a spacing of 5 cm. Panel P of FIG. 2 shows the time-resolved responses of BioSUM1 at different locations, indicating expected performance and a spatial resolution comparable to the sizes and spacings of the devices.

TABLE 1
All diffusivity constants

| | Dₗ | Dₕ | J_{mid} | J_{range} |
|---|---|---|---|---|
| BioSUM1 | 2.65 × 10⁻¹⁰ m²/s | 0.02Dₗ | 2 | 1.5 |
| BioSUM2 | 2.65 × 10⁻¹⁰ m²/s | 0.02Dₗ | 2.5 | 2 |
| BioSUM3 | 1.93 × 10⁻⁹ m²/s | 0.02Dₗ | 3 | 2 |

TABLE 2
Mechanical characteristics of pH-responsive BioSUM
materials after fully swollen in PBS solution.

| | Young's modulus (MPa) | Elongation at break (%) |
|---|---|---|
| BioSUM1 | 0.92 ± 0.06 | 112 ± 9 |
| BioSUM2 | 0.11 ± 0.01 | 66 ± 7 |
| BioSUM3 | 0.67 ± 0.04 | 220 ± 11 |

TABLE 3
Mechanical characteristics of pH-responsive BioSUM
materials after fully swollen in simulated fluids.

| | Young's modulus | Elongation at |
|---|---|---|
| | (MPa) | break (%) |
| BioSUM1 after swollen in SGF | 0.25 ± 0.03 | 23 ± 2 |
| BioSUM2 after swollen in SIF | 0.10 ± 0.01 | 60 ± 2 |
| BioSUM3 after swollen in SPJ | 0.36 ± 0.03 | 156 ± 8 |

TABLE 4
Mechanical characteristics of pH-responsive hydrogels
with different DMAEMA and DPAEMA ratios after
fully swollen in the PBS solution.

| | Young's modulus | Elongation at break |
|---|---|---|
| | (MPa) | (%) |
| 6:4 | 2.06 ± 0.09 | 147 ± 7 |
| 7:3 (BioSUM1) | 0.92 ± 0.06 | 112 ± 9 |
| 8:2 | 0.45 ± 0.01 | 95 ± 1 |
| 9:1 (BioSUM2) | 0.11 ± 0.01 | 66 ± 7 |
| 10 | 0.040 ± 0.001 | 35 ± 8 |

TABLE 5
Mechanical characteristics of pH-responsive hydrogels with different
AAc and PEGDA ratios after fully swollen in the PBS solution.

| | Young's modulus | |
|---|---|---|
| | (MPa) | Elongation at break (%) |
| 2× AAc | 1.04 ± 0.02 | 144 ± 11 |
| 0.5× AAc | 0.31 ± 0.02 | 260 ± 8 |
| 1× AAc, 1× PEGDA | 0.67 ± 0.04 | 220 ± 11 |
| (BioSUM3) | | |
| 2× PEGDA | 0.84 ± 0.04 | 172 ± 7 |
| 0.5× PEGDA | 0.53 ± 0.01 | 255 ± 9 |

### Sensing by Ultrasound Imaging in Deep Tissues

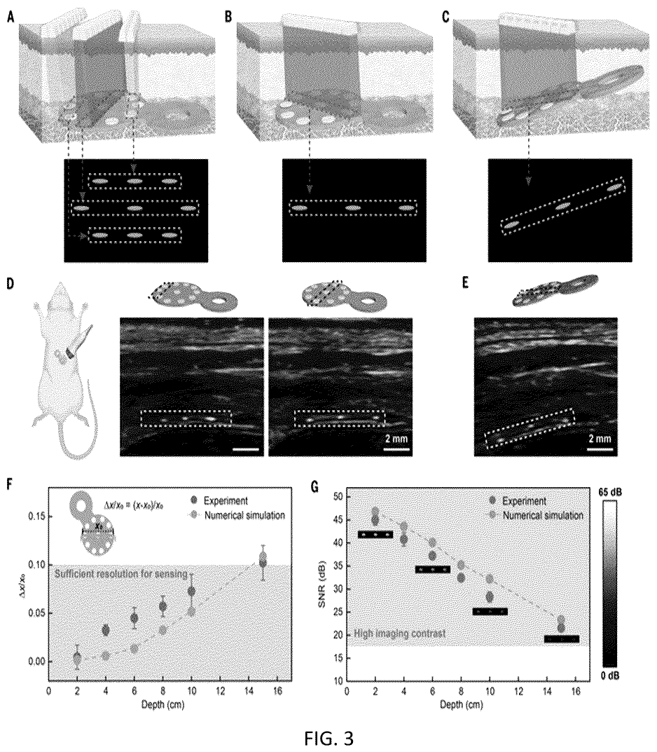

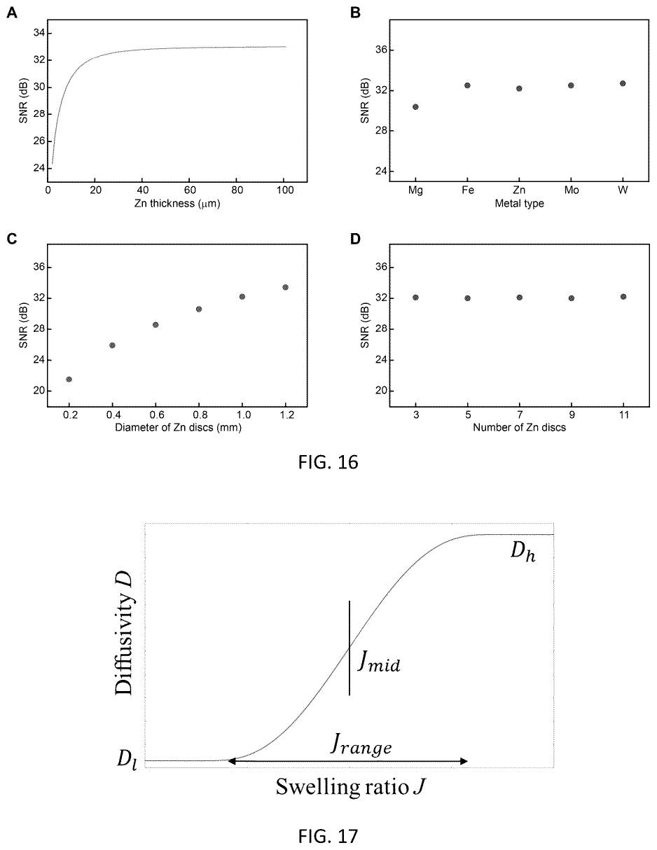

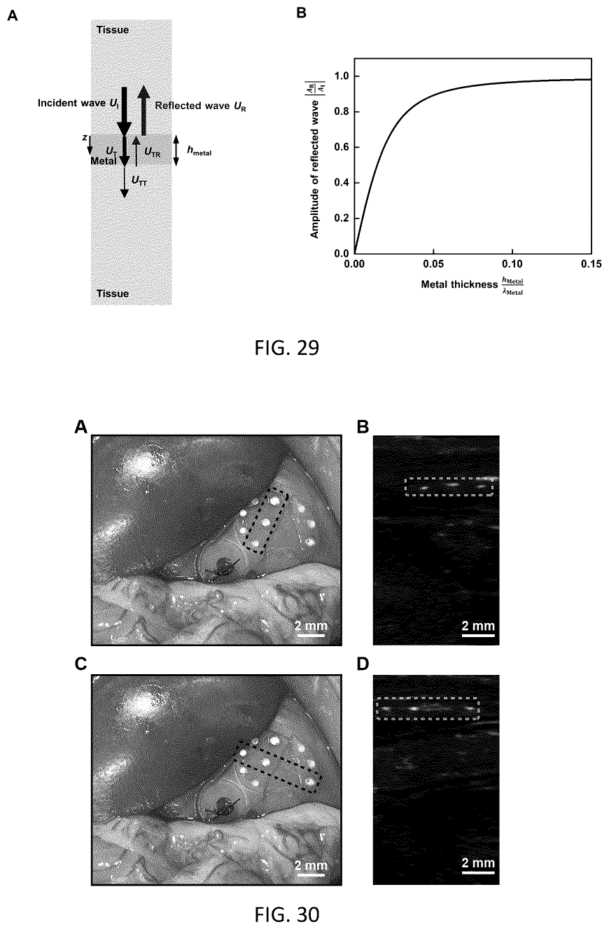

Ultrasound B-mode imaging serves as a mechanism for quantitatively evaluating the pH-dependent geometry of these responsive hydrogels when implanted at deep tissue locations. The discs of Zn, an established bioresorbable metal that reacts with water to yield the benign end product of Zn(OH)₂, act as ultrasonic indicators. The acoustic impedance mismatch between these Zn structures and the surrounding hydrogel matrix and adjacent soft tissues enhances their visibility in B-mode images. The reflection coefficient is approximately proportional to (Zi-Z₂)²/(Z₁+Z₂)² for structure thickness greater than ~20 μm for normal incidence specular reflections, where Z₁ and Z₂ are the impedances for the Zn and the hydrogel (see FIGS. 16 and 29 for details about reflections for thicknesses less than 20 mm). For the systems reported here, the reflection coefficients at the interface between the Zn structures and the hydrogel are greater than 80%. By contrast, coefficients between typical surrounding soft tissues and the hydrogel are less than 0.5%. The symmetric, circular distribution of these discs (panels A-C of FIG. 3) allows ultrasonic visualization in a manner that is independent of the orientation. During an ultrasound scan, acoustic waves generated by the transducer penetrate through tissues and reflect from these discs to form a cross-sectional image of their spacings. For the specific geometry presented here, the array of discs appears as three equally distributed bright segments in the image, clearly differentiated from background features associated with tissue structures. The distance between these hyperechoic components may shift in ultrasound images, depending on the specific cross-sectional position and orientation of the acoustic waves with respect to the device (panel A of FIG. 3). The cross-sectional position across the diameter of the device represents the largest separation distance between bright segments in B-mode imaging, which defines the actual changes in dimension. An example of this process conducted in a rat model with a device on the surface of the stomach appears in panel D of FIG. 3. The left image denotes signals of BioSUM on the perimeter, and the right image shows signals from three discs along the diameter.

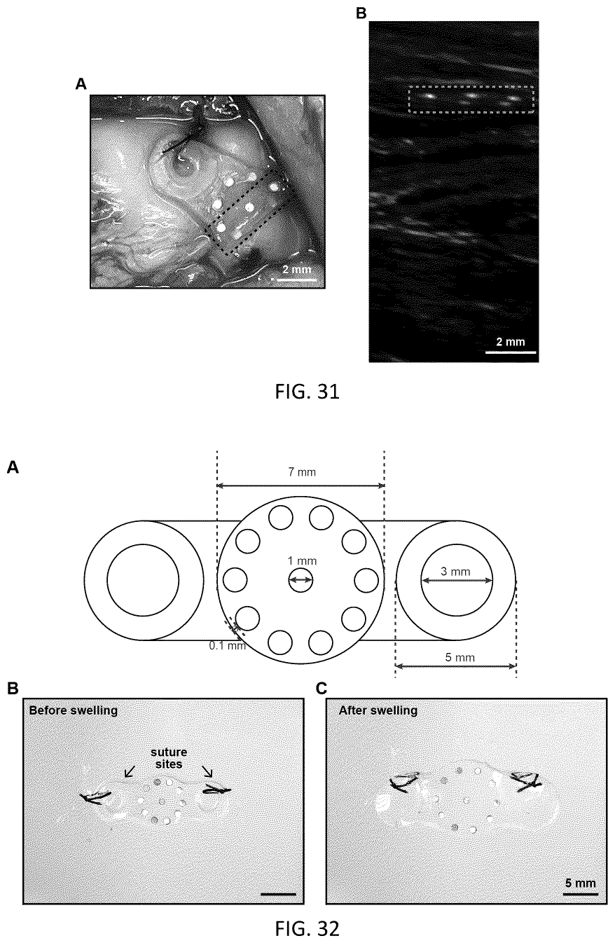

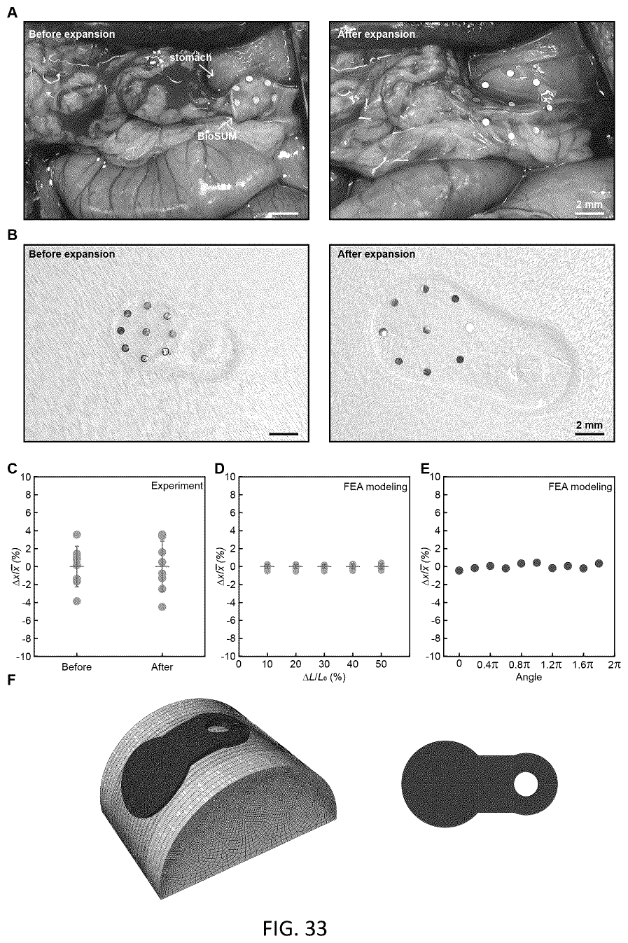

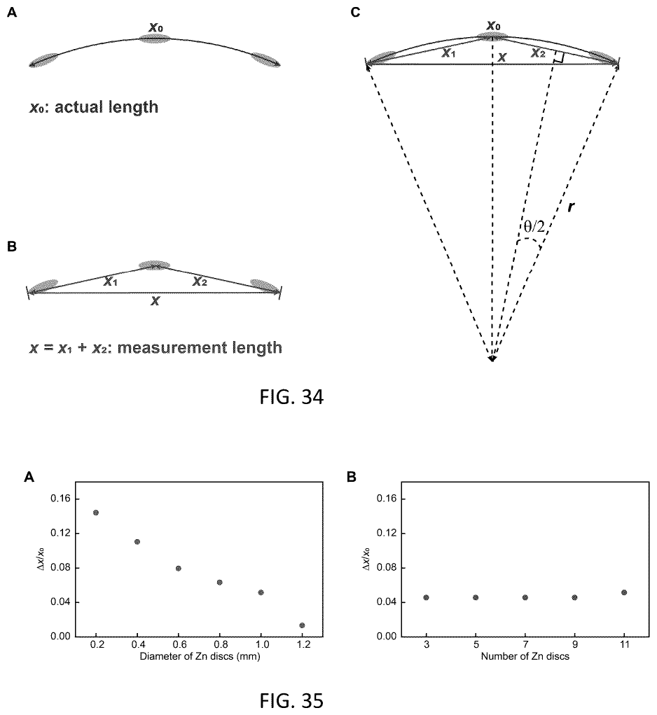

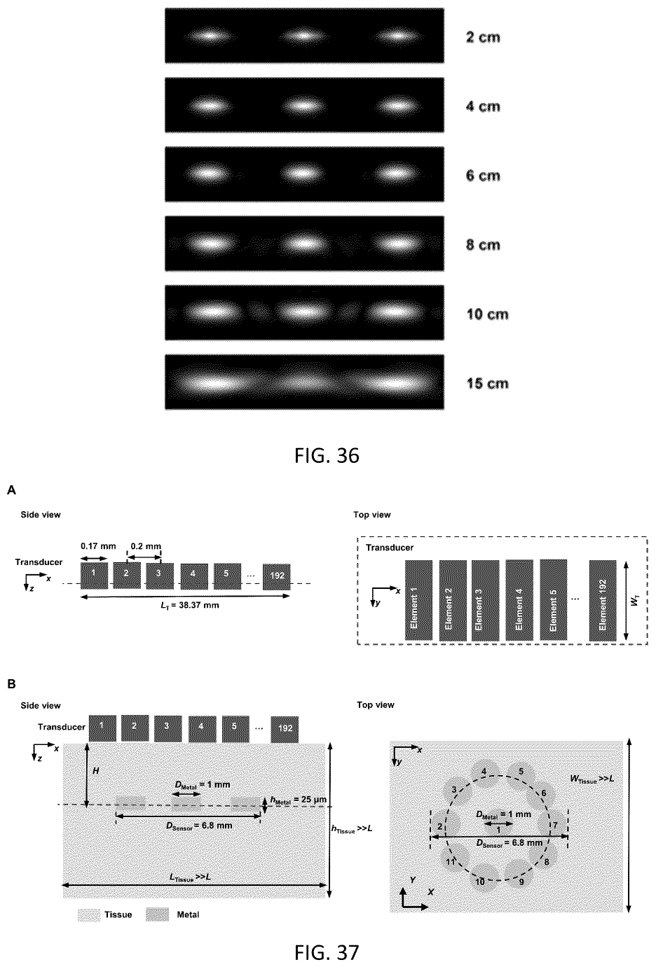

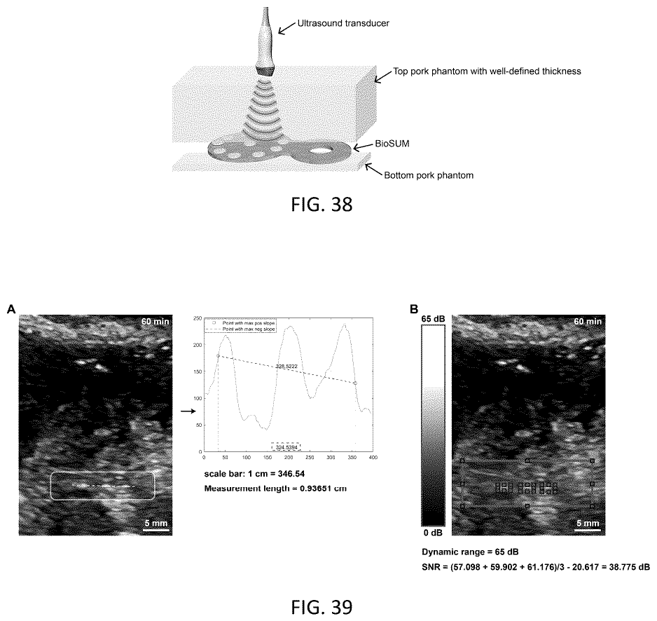

The symmetric design of the discs and the mechanically flexible nature of the BioSUM enable detection in non-planar configurations (FIGS. 30-32). For example, when the BioSUM is not parallel to the transducer, it appears tilted in the B-mode image, yet still reflects signals from the three discs along the diameter (panel C of FIG. 3). The natural curvature of the targeted organs may lead to such tilted orientations (panel E of FIG. 3) but without an effect on the measured distances between the discs, as in the right image of panel D of FIG. 3. Additional details are shown in FIGS. 33-34.

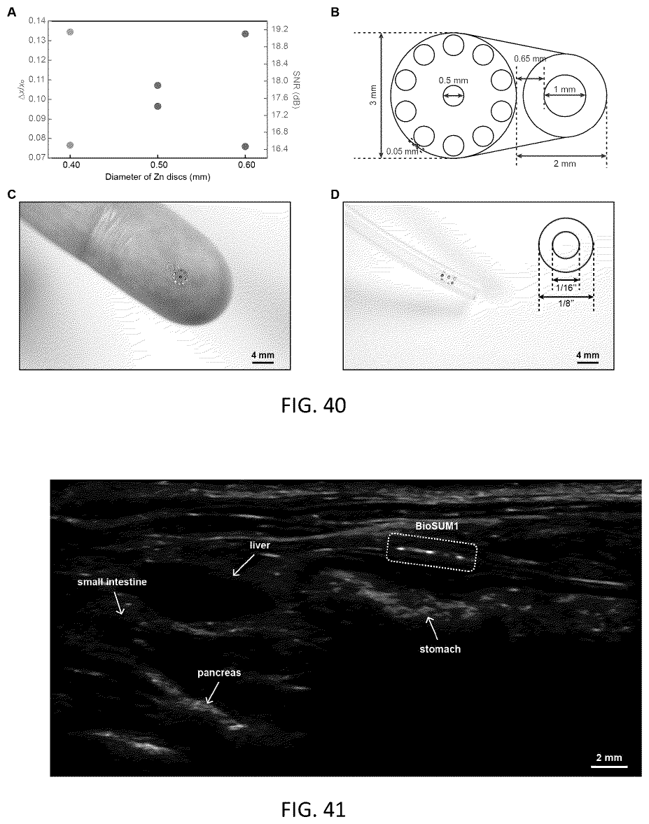

The signal-to-noise ratio (SNR) and the lateral resolution in B-mode images quantify the signal quality, yet both properties diminish with depths due to the attenuation of ultrasonic waves as they propagate through the tissues. Lateral resolution (in terms of absolute length) is proportional to λF/L, where λ is the ultrasound wavelength, F is the focal depth, and L is the aperture length. With a fixed transducer (i.e., fixed aperture length) and scanning frequency (i.e., ultrasound wavelength), the resolution decreases with depth. Scattering from heterogeneous tissues causes attenuation that deteriorates the SNR as the depth increases. Experimental and numerical investigations (panels f-g of FIG. 3 and FIGS. 16, 35-39) quantify the depth dependence of the signal quality using ultrasonic waves with frequencies of 5 MHz. The experimental and numerical simulation results show similar trends over depth. The slightly lower signal quality from experiments may be attributed to the effects of the inhomogeneities in the soft tissues. The deviation of the measured separation between Zn discs divided by the actual separation, i.e. Δx/x₀, serves as a relevant metric. Numerical simulation and experimental results for images captured underneath a pork phantom in panel F of FIG. 3 show that Δx/x₀ increases with depth but remains less than 0.1 for depths up to 15 cm. This result indicates that dimensional changes of 10% or more can be accurately detected at depths of 15 cm, consistent with the swelling behaviors of the devices reported here. For SNR defined as the average contrast of three bright segments relative to the background, the results indicate that the SNR remains above 18 dB for depths of up to 15 cm (panel G of FIG. 3), necessary for clear visualization in medical imaging. The SNR and Δx/x₀ deteriorate as the diameter of the Zn discs decreases (FIGS. 16 and 35), as a limiting consideration in miniaturizing these structures. Simulation results indicate that to achieve Δx/x₀~0.1 and SNR~18 dB, the hydrogel and discs must have diameters of 3 mm and 0.5 mm, respectively (FIG. 40).

### Monitoring pH Homeostasis in Animal Models

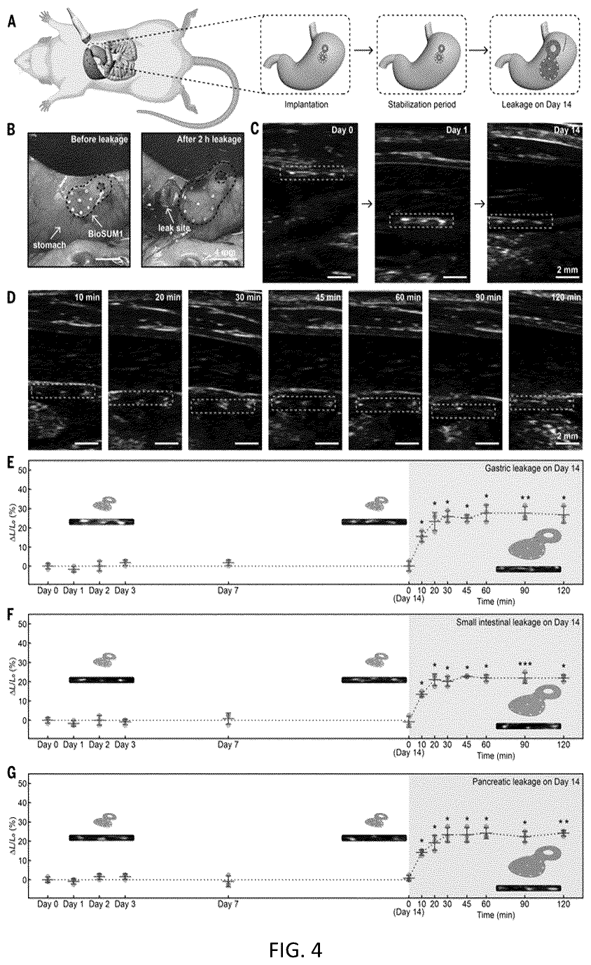

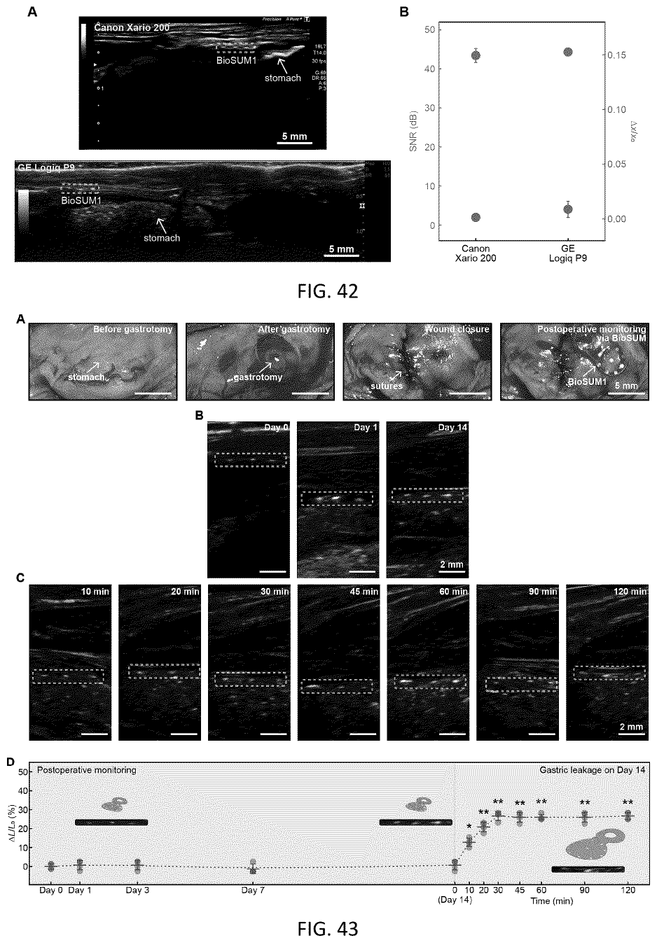

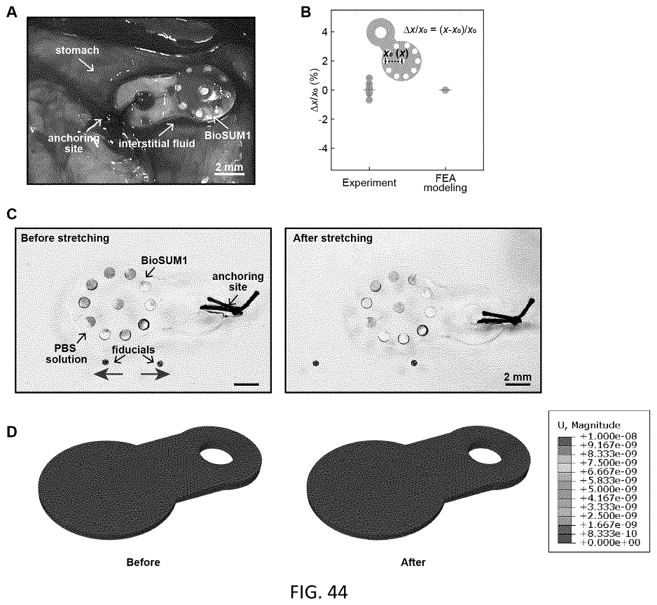

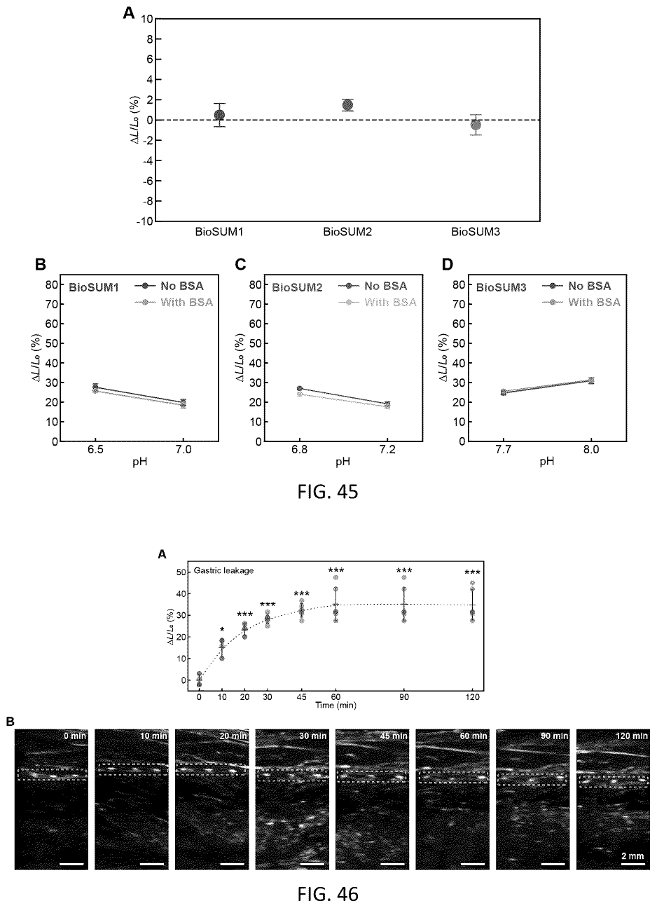

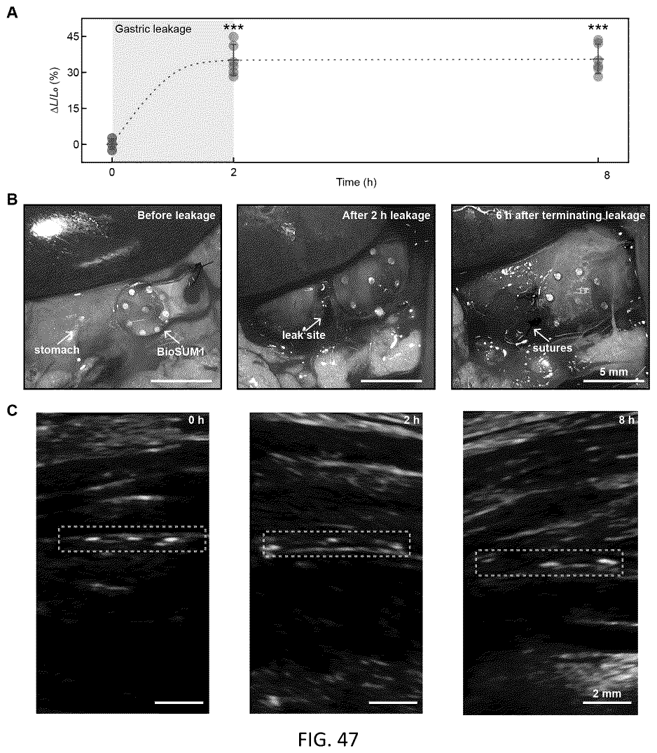

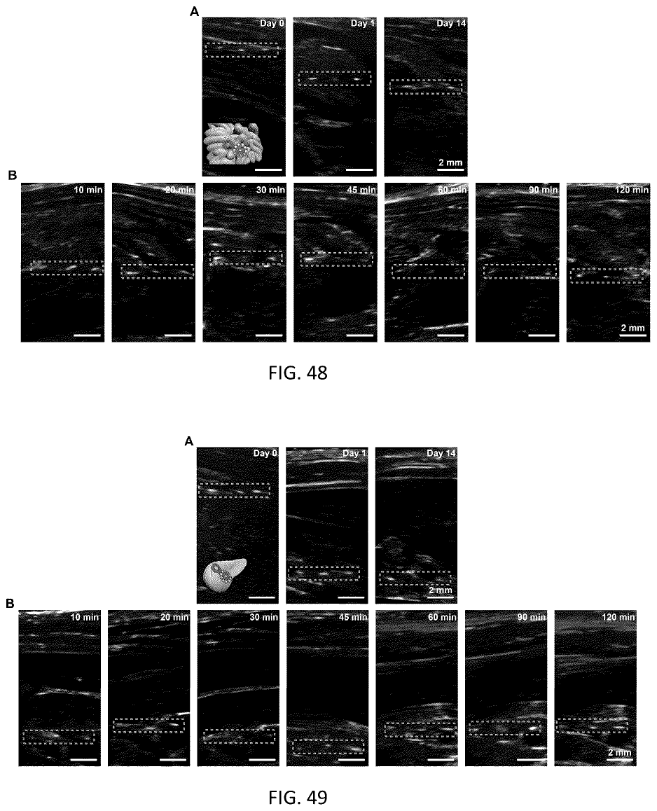

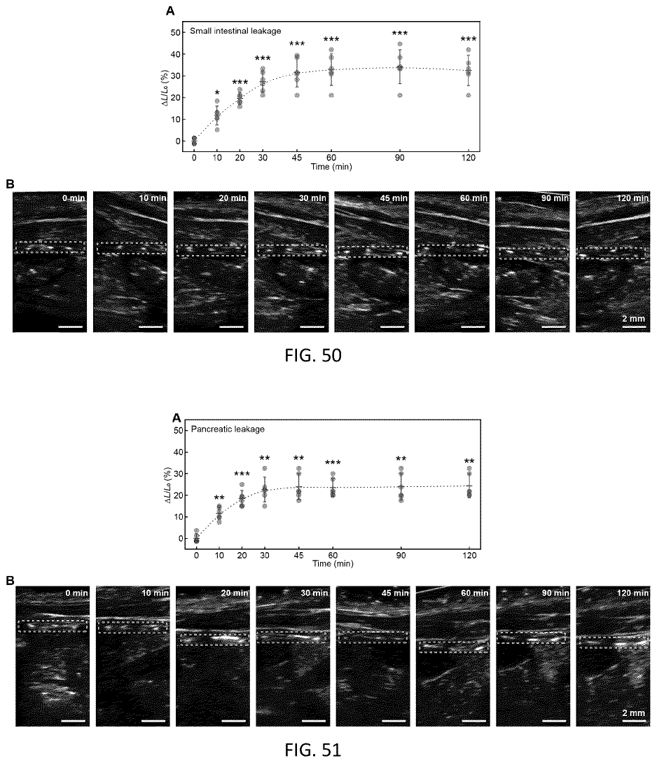

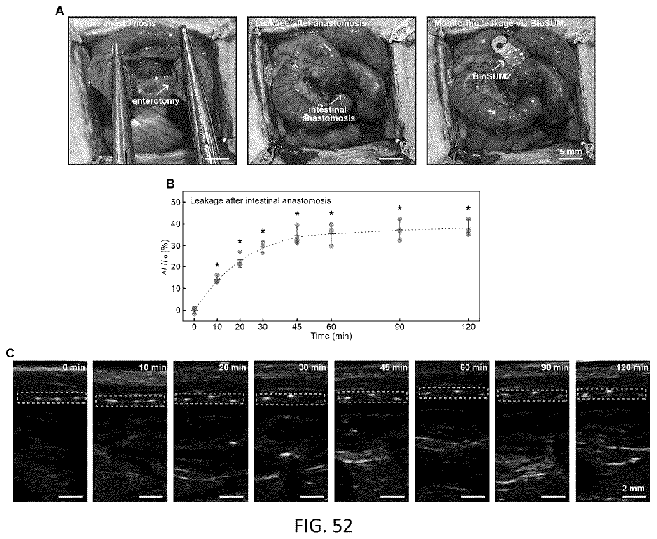

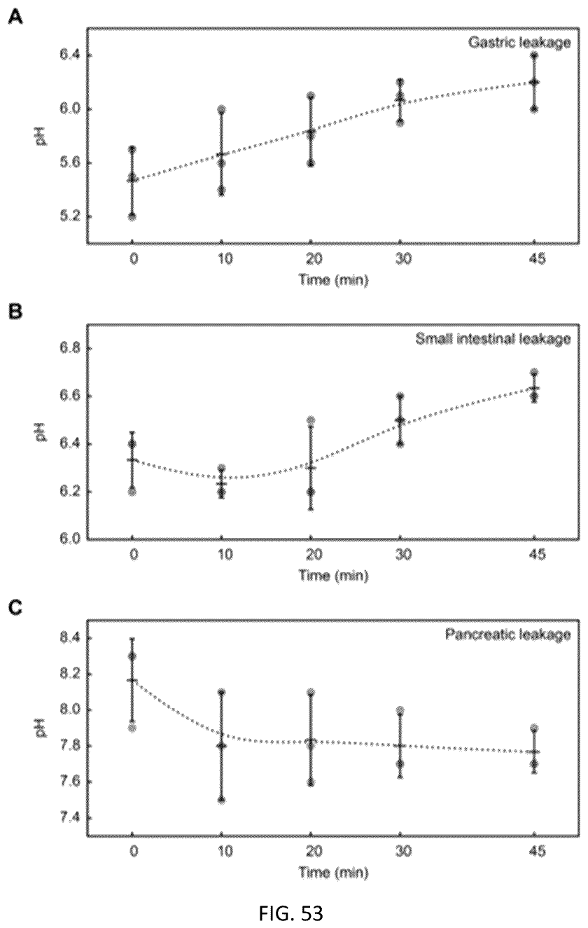

Longitudinal measurements using rat models validate the feasibility of ultrasonic detection of GI leakage, compatible with various ultrasound imaging systems (FIGS. 41-42). The studies involve imaging after implantation on the stomach and for a subsequent 14 days to confirm the geometric and operational stability in this physiological environment. Creating a 3-mm gastrotomy (incision in the stomach) on Day 14 leads to leakage, monitored immediately after closing the surgical site by imaging for a subsequent 2 h (panel A of FIG. 4, and FIG. 43). Visual inspection afterward confirms the geometric changes inferred from the imaging results (panel B of FIG. 4). The results show minimal fluctuations in dimensions on postoperative days 1 through 14 (FIG. 44), consistent with an absence of movements of the device and with a local pH stably maintained by homeostatic regulation (panel C of FIG. 4, and FIG. 45). Following the creation of a gastrotomy on postoperative day 14, the BioSUM1 expands gradually during the 2 h period, evident by the increased distance between the Zn discs in B-mode images (panel D of FIG. 4). The swelling is evident 10 min after the gastrotomy, (panel E of FIG. 4, and FIG. 46), even for the modest volume (<1 mL) of gastric fluid released in these experiments. In addition, the expanded dimensions of the device after swelling remain stable after 2 h. Further in vivo experiments confirm this behavior for at least 6 h after leakage terminates (FIG. 46). Specifically, 2 h after terminating leakage and closing the surgical site, the rats move freely for 6 h. Ultrasonic evaluation after this period indicates that the dimensions are unchanged. In clinical practice, this behavior may prevent false-negative readouts that may otherwise arise due to delayed examinations. Similar procedures validate capabilities for ultrasound imaging of changes in pH associated with small intestinal and pancreatic leaks using the alternative hydrogel chemistries described previously (panels F-G of FIG. 4, and FIGS. 48-51). Real-time, dynamic changes in the pH of the local environment associated with leaks in the rat model obtained from benchtop studies of swelling behaviors appear in FIG. 53.

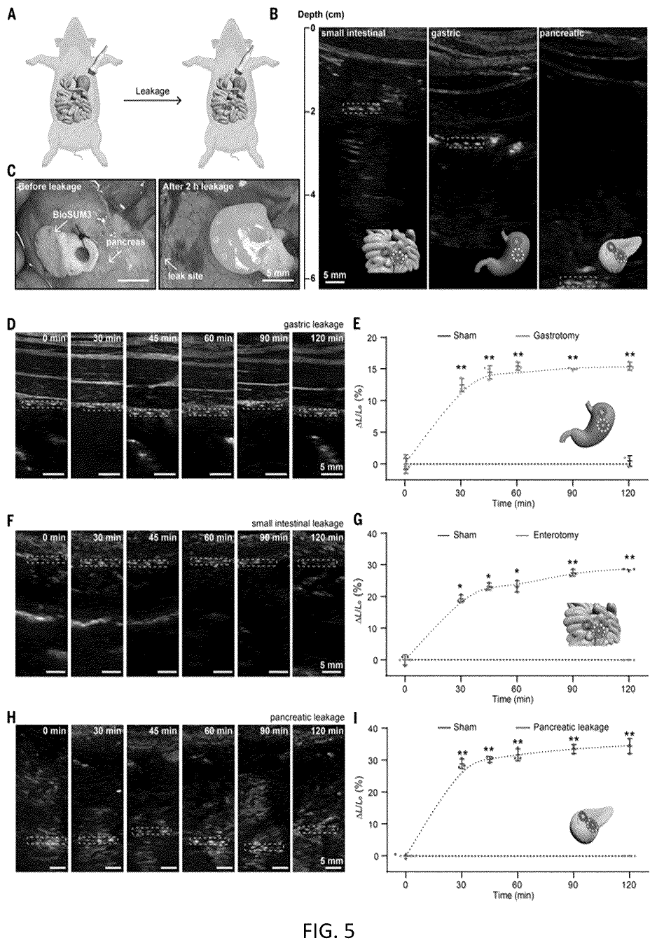

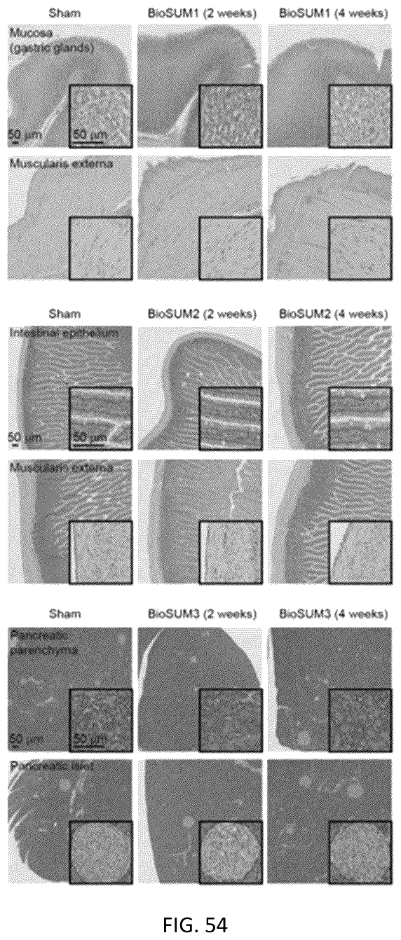

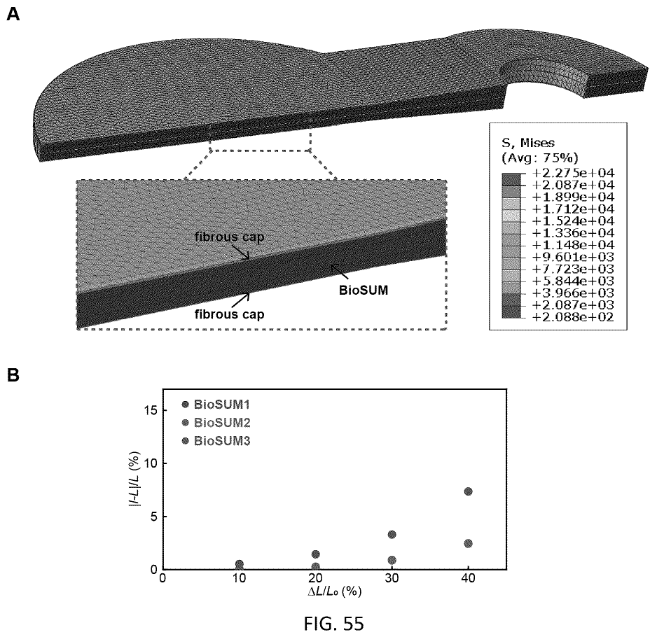

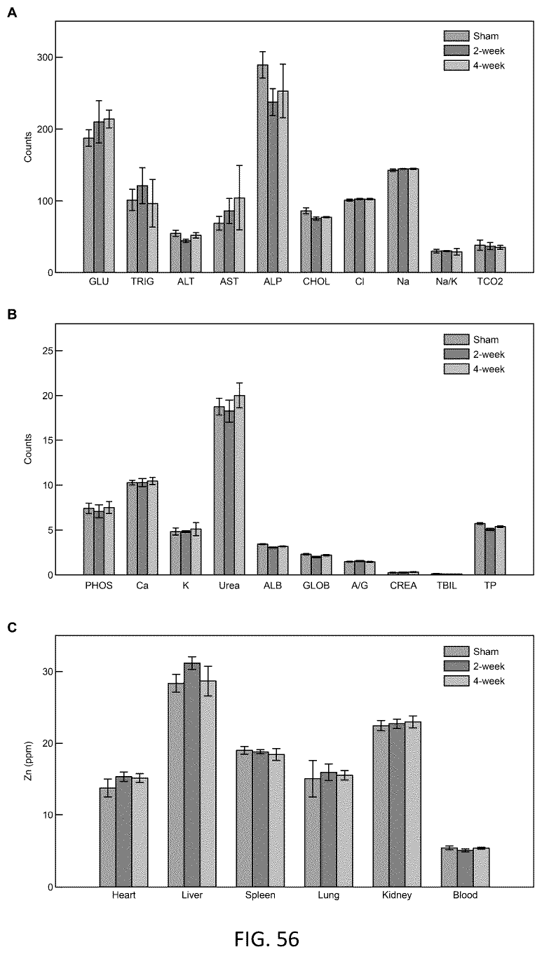

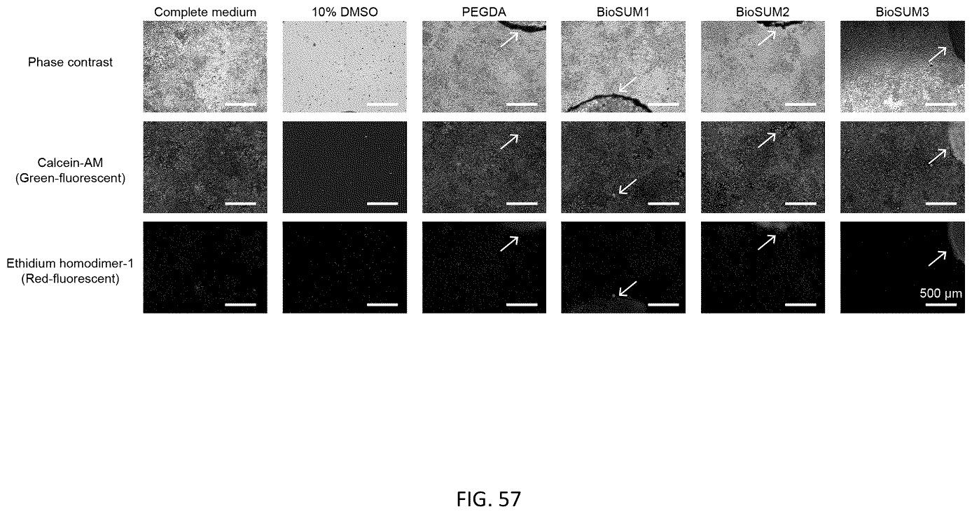

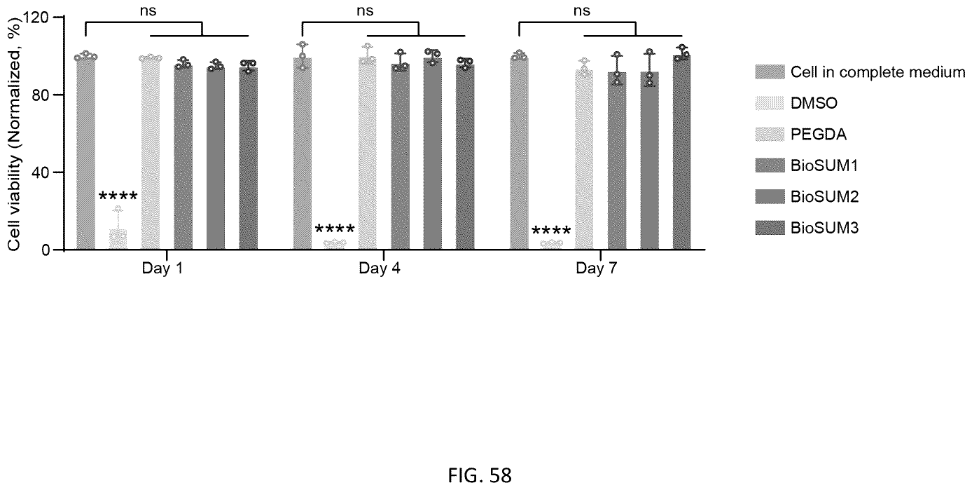

Tests on porcine models confirm operation at scales comparable to the adult human GI tract (panel A of FIG. 5). The results in panel B of FIG. 5 successfully demonstrate ultrasonic detection of BioSUMs on GI organs, imaged transabdominally through multiple layers of tissues with heterogeneous acoustic properties. Across this range of depths, the Zn discs array appears as distinct features distinguishable from the background. Inducing GI leakage adjacent to the devices enables monitoring of changes in a subsequent 2 h period. Visual inspection confirms these dimensional changes for the case of 2 h after the creation of a pancreatic leak (panel C of FIG. 5). The first ultrasonic scan, performed 30 min after creation of the GI leak, reveals significant dimensional changes in all three types of BioSUMs. The average swelling ratio is >10% 30 min after the creation of the leak (panels D-I of FIG. 5). Histological analysis shows no remarkable inflammation, fibrosis, or necrosis compared to a sham group (FIG. 54-55). Evaluations of blood chemistry indicate results within normal ranges (panels A-B of FIG. 56), and element analysis shows no significant accumulation of Zn in the heart, liver, spleen, lung, kidney and blood, both compared to the sham group (panel C of FIG. 56). In vitro cytocompatibility of BioSUMs reveal negligible effect on cell viability and no significant cell death in close proximity to the devices (FIGS. 57-58).

### Example 2

### Materials and Methods

Synthesis of Bioresorbable, pH-Responsive Hydrogels

The synthesis of BioSUM1 started with the chain extension reaction of a mixture of 2-(dimethylamino)ethyl methacrylate monomer (DMAEMA, Sigma Aldrich) and 2-(diisopropylamino)ethyl methacrylate monomer (DPAEMA, Sigma Aldrich) (DMAEMA:DPAEMA=7:3 in weight ratio). Adding 2 wt % 2-hydroxy-2-methylpropiophenone photoinitiator (Darocur 1173, Sigma Aldrich) followed by UV irradiation (365 nm, 500 mW/cm²) for 8 min yielded DPAEMA-DMAEMA oligomers. Subsequent addition of 3.85 wt % poly(ethylene glycol) diacrylate (PEGDA, Mₙ 250, Sigma Aldrich) with respect to the monomer mixture and 0.5 wt % 2,2-dimethoxy-2-phenylacetophenone photoinitiator (DMPA, Sigma Aldrich) formed a precursor to the hydrogel. UV exposure for 8 min under a nitrogen atmosphere yielded a crosslinked hydrogel network.

Synthesis of BioSUM2 started with the chain extension reaction of a mixture of DMAEMA and DPAEMA (DMAEMA:DPAEMA 9:1 in weight ratio). Adding 2 wt % Darocur 1173 followed by UV irradiation (365 nm, 500 mW/cm²) for 8.5 min yielded DPAEMA-DMAEMA oligomers. Subsequent addition of 2.9 wt % PEGDA Mₙ 250 and 0.5 wt % DMPA photoinitiator formed a precursor to the hydrogel. UV exposure for 10 min under a nitrogen atmosphere yielded a crosslinked hydrogel network.

Synthesis of BioSUM3 started with the chain extension reaction of butyl acrylate monomer (BA, Sigma Aldrich). Adding 0.5 wt % Darocur 1173 photoinitiator followed by UV irradiation (365 nm, 500 mW/cm²) for 160 s yielded the BA oligomer. Subsequent addition of 21.8 wt % of the pH-responsive monomer acrylic acid (AAc, Sigma Aldrich), 1 wt % PEGDA Mn 250 and 0.5 wt % DMPA photoinitiator formed a precursor to the hydrogel. UV exposure for 10 min under a nitrogen atmosphere yielded a crosslinked hydrogel network.

### Fabrication of Bioresorbable, Shape-Adaptive Ultrasound-Readable Materials Structures (BioSUMs)

Fabrication of BioSUMs for the porcine model began with blade coating of hydrogel precursor and partial curing into a 150 μm thick film (100 μm thick for the rat model) by UV exposure under a nitrogen atmosphere on a glass slide treated with 0.2 vol % trichloro(octadecyl)silane (Sigma Aldrich) hexane solution to ensure easy detachment after fabrication. Laser cutting (LPKF Protolaser R) of Zn foils (25 μm thick, Goodfellow, USA) into 1 mm in diameter (500 μm in diameter for the rat model) disks formed ultrasonic indicators. Assembling the Zn disks into the designated symmetric pattern onto the partially cured hydrogel film and subsequently blade coating an additional 150 μm thick hydrogel layer (100 μm thick for the rat model) enclosed the disks in the middle. Punching the hydrogel into desired disks using a hollow hole punch with preset diameter defined the sensing part of the BioSUM1 and BioSUM2. Forming an additional coating of −10 μm p(DPAEMA-DMAEMA) hydrogel layer onto BioSUM3 completed the sensing part of BioSUM3. The diameter of the sensing part for BioSUM1 was 7 mm (4 mm for the rat model), and that for BioSUM2 and BioSUM3 was 6 mm (3.5 mm for the rat model). Punching a hydrogel film of 500 μm thickness (250 μm thick for the rat model) into a 5 mm diameter disk with a 2 mm diameter hole (3 mm diameter disk with 1 mm diameter hole for the rat model) formed the suture ring. Assembly of the sensing part and the suture ring using an additional layer of hydrogel precursor followed by UV irradiation under a nitrogen atmosphere completed the fabrication.

### Acquisition of Ultrasound B-Mode Images

A medical ultrasound instrument (GE LOGIQ P9, GE Healthcare) paired with a transducer (C1-5 broad-spectrum convex array transducer, 5 MHz frequency, GE Healthcare, unless otherwise stated) yielded images for the in vitro and in vivo tests. Applying ultrasound gel (MediChoice ultrasound gel, MAC Medical Supply Co, USA) at the interface, manually adjusting the gain, dynamic range, time gain compensation (TGC), and setting the focal zone at the depth of the BioSUM enabled capture of high-quality B-mode images. The measurement function of the instrument yielded the distance between the left and right bright segments corresponding to the Zn disks in the BioSUM. The maximum rate of decrease in the image contrast at these segments, as judged by the operator and validated by an automated image analysis algorithm (MATLAB coding), defined the positions of their edges. Post-processing of images involved coloring the grayscale images to yield the final B-mode images by the Lookup Table in ImageJ. The ultrasonic detection of BioSUMs in an alternative imaging system adopted the ultrasound instrument (Canon Xario 200) paired with a 18L7 transducer.

### Finite Element Analysis (FEA) of Hydrogel Swelling

The commercial software COMSOL 6.0 was used to perform the coupled mechanical-transport FEA to establish the swelling ratio as a function of time. One-quarter of the device was modeled due to its symmetry. Second-order elements were implemented for displacements, while first-order elements were employed for the chemical potential of water. Initially, equilibrium swelling ratios were computed through steady state simulations to get the Flory-Huggins parameters at different pH levels (as detailed in the supplementary text). Subsequently, time-dependent simulations were applied to compute the swelling ratio curves at different pH levels across the three types of hydrogels after fully swollen in pH 7.4, thereby facilitating the determination of diffusivity constants.

### Ultrasonic Determination of the Swelling Behaviors

The system described above yielded data on the time-resolved swelling of BioSUM upon exposure to changes in pH. Immersion in phosphate buffered saline (PBS, pH 7.4, Sigma Aldrich) for 24 h prior to the tests stabilized the dimensions of the BioSUM. Immersion in pH buffer solutions for designated time intervals and followed by placement between two pork phantoms (pork loin chops) with different thicknesses (2-cm thick top layer for the rat model and 6-cm for the porcine model) to simulate the in vivo detection environment. Manually moving the ultrasound transducer across the top surface, adjusting the angles and targeting signals from the three dots along the diameter yielded images for analysis. Measuring the distances as described above allowed determination of the swelling ratio in length (ΔL/L₀%). Each data point in the plot corresponds to measurements from 3 samples.

### Direct Measurements of the Sensitivity

As described above, immersion in PBS (pH 7.4) for 24 h preceded the introduction of designated amounts (0.1 mL, 0.2 mL, 0.5 mL, 1 mL, and 2 mL) of simulated gastric fluid (pH 1.2, Ricca Chemical Company), simulated intestinal fluid (pH 6.8, Ricca Chemical Company), and simulated pancreatic fluid (pH 8.2, Biochemazone) directly onto BioSUM1, BioSUM2, and BioSUM3 in evaluations of their sensitivity. Optical measurement of the distances between disks on opposite ends at 1, 3, 5, 10, 15, 20, 25 and 30 min, and converting the results into swelling ratios in length (ΔL/L₀%) defined the sensitivity. Linear fitting of ΔL/L₀% determined the response time to reach a swelling ratio of 10%. Each data point in the plot corresponds to measurements from 3 samples.

### Demonstrations of Spatio-Temporal Sensing

The experiments involved introducing 250 mL PBS (thickness=1.25 cm) into a tank (40 cm in length, 5 cm in width) with a substrate of 1% (w/v) agarose gel. Placing 7 BioSUM1s (pre-soaked in PBS for 24 h prior to the tests) along a line with a separation distance of 5 cm and then introducing 15 mL simulated gastric fluid (SGF) at one side led to a spatiotemporally varying pH, dictated by convection. This set up aims to resemble the in vivo situations that involve small leaks (~3 mL) by scaling the total amount of fluid, as the normal fluid amount in the peritoneal cavity is between 50 and 75 mL. Optically recording the swelling ratio of each BioSUM1 at time intervals of 5 min for up to 30 min produced a set of values relative to those of the BioSUM1 located at 0 cm at 30 min. Each data point in the plot corresponds to 3 independent measurements.

### Tests of Biodegradation

Immersion of BioSUM into PBS (pH 7.4) at 37° C. and 95° C. tested the biodegradation. The PBS solution was changed every two days.

Gravimetric analysis served to quantify the biodegradation of hydrogel films without Zn disks. Soaking pre-weighed hydrogel samples in PBS (pH 7.4) at 37° C., 75° C. and 95° C., washing the samples with deionized water to remove residual salts, and drying in a vacuum oven on designated days enabled measurements of dry weights. The ratio of the dry weight to the initial weight defined the percentage of the remaining hydrogels. The PBS solution was changed every two days.

### Characterization of Mechanical Properties

Dynamic mechanical analysis (DMA, RSA-G2 Solids Analyzer, TA Instruments) defined the stress-strain relationships in uniaxial tension tests with a constant moving speed of 5 mm/min. Measurements included hydrogel films without Zn disks (5 mm in width, 10 mm in length, 300 μm in thickness, pre-swollen in the PBS solution or simulated fluids for 24 h). Mechanical properties from each BioSUM were measured 3 times.

The measurement of wear rates used sandpaper of 400 grit (Gator & Co) to press onto the hydrogel material. Moving the paper back and forth for a total of 100 cycles wore the hydrogel. The weight of the hydrogel was measured every 10 cycles.

### Numerical Simulation of Ultrasound B-Mode Imaging

The wave propagation and ultrasound B-mode imaging were simulated by MATLAB ultrasound toolbox MUST. The model consisted of the transducer, the tissue and the sensor (panel A of FIG. 37). The transducer consisted of 192 elements (length=0.17 mm for each element) that can induce and receive pressure waves independently. The pitch, i.e. distance between adjacent elements, was 0.2 mm such that the total length of the transducer was L_{T}=38.37 mm. The transducer was placed at the top of the tissue to induce acoustic waves. The tissue length, width and thickness were much larger than the transducer. The acoustic wave velocity and density of tissue were c_{Tissue}=1540 m/s (39) and ρ_{Tissue}=1 g/cm³, respectively, such that the acoustic impedance was z_{Tissue}=c_{Tissue}ρ_{Tissue}=1.54 MPa s/m. The device consisted of N disk-shaped metal disks of diameter D_{Metal}, thickness h_{Metal} and acoustic impedance z_{Metal}, with one metal disk at the center and the rest uniformly patterned on the perimeter. The diameter of the device, defined as the distance between the edges of metal disks at opposite ends (panel B of FIG. 37), was much smaller than the transducer length L_{T} and width W_{T}. The hydrogel that enclosed the metal disks in the experiment was considered as the same material as the tissue, because its acoustic wave velocity and density are very close to those of the tissue. The device was embedded in the tissue at a depth of H. In the simulation, the pressure received by the transducer elements was calculated by the functions in the toolbox based on the reflection coefficient

$$r = \vert \frac{{z}_{Metal - {Z}_{Tissue}}}{{z}_{Metal} + {z}_{Tissue}} \vert$$

at the tissue/metal interface. The background noise was simulated by randomly placing noise particles with reflection 1.0 and volume density 18.0/cm³ (number of particles per unit volume, fitted based on experimental results of the signal-to-noise ratio). The baseline values of the above parameters were D_{Metal}=1 mm, h_{Metal}=25 μm, H=10 cm, acoustics wave velocity and density of metal (Zn) c_{Metal}=2780 m/s and ρ_{Metal}=7.14 g/cm³ (z_{Metal}=c_{Metal}ρ_{Metal}=19.8 MPa s/m), respectively.

### Characterization of Measurement Accuracy

Characterization of measurement accuracy used BioSUM placed between two pieces of pork phantoms with various thicknesses of the top layer (2 cm, 4 cm, 6 cm, 8 cm, 10 cm and 15 cm). Placing the ultrasound transducer on the top surface captured BioSUM in ultrasound B-mode and yielded images of 3 equally distributed bright segments with the maximum distance in between. MATLAB (coding available from the authors upon request) extracted the pixel intensity plot of BioSUM, defined the edge of BioSUM as the maximum slope in the intensity plot and calculated the pixel distance in between (panel A of FIG. 39). Further conversion into the distance in the centimeter scale based on the depth scale bar of images provided the measurement length of BioSUM underneath different depths of the pork phantom. Deviation of measurement length divided by the ground truth length of BioSUM quantified the measurement accuracy. The measurement from 3 samples formed one data point in the plot.

### Characterization of Signal-to-Noise Ratio (SNR)

Similar to the characterization of measurement accuracy, the characterization of signal-to-noise ratio (SNR) began with placing BioSUM between two pieces of pork phantoms with the thickness of the top layer varying from 2 cm to 15 cm. Obtaining 3 equally distributed bright segments with the maximum distance in between represented signals of BioSUM in ultrasound B-mode. A rectangle of 0.2 cm×0.4 cm, including the bright segment, defined the regions of interest (ROIs) of signals from BioSUM, and a rectangle of 3 cm long x 1 cm high around the same depth of BioSUM defined the ROI of the background. Performing linear regression based on the dynamic range of individual image converted pixel intensity into decibel (dB) units. Extracting the average grayscale values from signals with background subtraction and further conversion into contrast in dB unit quantified SNR values at different depths (panel B of FIG. 39). The measurement from 3 samples formed one data point in the plot.

### Implantation in Rodent Models

The experimental procedures with rat models followed approvals from the Institutional Animal Care and Use Committees at Washington University in St. Louis (protocol #22-0022). Adult male Lewis rats (8-10 weeks old) were purchased from Charles River Laboratories, Wilmington, MA. All rats were provided with food (PicoLab rodent diet 20, Purina Mills Nutrition International, St. Louis, MO) and water ad libitum. The surgical procedure started with sterilization using betadine and isopropanol solutions, and anesthesia with inhaled isoflurane vapor (4% for induction and 2% for maintenance) during the implantation surgery. Placing the rats in the supine position and performing laparotomy with a 3-cm midline incision on the abdomen exposed related organs. Gently exposing the stomach with atraumatic forceps allowed for implantation of BioSUM1 (pre-soaked in PBS at a pH of 7.4 for 24 h prior) by securing it with sutures via the ring. Similarly, exposing the intestine gently by holding it with atraumatic forceps facilitated the implantation of BioSUM2 (pre-soaked in PBS at a pH of 7.4 for 24 h prior). Exposing the pancreas enabled implantation of BioSUM3 (pre-soaked in PBS at a pH of 7.4 for 24 h prior), which included procedures of exteriorizing the duodenum and spleen by holding the stomach with atraumatic forceps, mobilizing the stomach and spleen through omentectomy, mobilizing the area between the colon and pancreas, and exposing the portal vein.

Ultrasound images of BioSUM captured using a 15 MHz transducer (ML6-15 broad-spectrum linear matrix array transducer, 15 MHz frequency, GE Healthcare) immediately after implantation and abdominal closure defined the results for Day 0. Similar measurements on Day 1, Day 2, Day 3, Day 7, and Day 14, each collected while the rats were under anesthesia, defined a stabilization period with no expected changes in pH. Postoperatively, all rats were monitored for signs of infection and distress daily, and no laparotomy was performed during the 14-day period. Disruption of gastrointestinal organs on Day 14 simulated anastomotic leakage. 3-mm gastrotomy on the front wall of the stomach and enterotomy on the wall of the small intestine initiated gastric and intestinal leakage, respectively, in separate experiments. Resection of the tail of the pancreas initiated pancreatic leakage. The rats were euthanized with pentobarbital (150 mg/kg) after monitoring the leakage by ultrasound for 2 h.

### Implantation in Porcine Models

The experimental procedures with porcine models followed approvals from the Institutional Animal Care and Use Committees at Northwestern University and Washington University in St. Louis (protocol #23-0028). The studies involved using both sexes of domestic swine (Sus domesticus) weighing 100-110 lbs. Anesthesia procedures included sedating the animals, placing them in a dorsal recumbent position, followed by intramuscular administration of telazol (4 mg/kg), ketamine (2 mg/kg), and xylazine (2 mg/kg). Using supplemental oxygen with isoflurane (inhalation at 1-5%) maintained anesthesia, while monitoring body temperature rectally and peripherally, and monitoring oxygen saturation and heart rate via a pulse oximeter.

Laparotomy began with placing animals in a supine position. Placing BioSUM1 on the stomach body, BioSUM2 on small intestines, and BioSUM3 on the pancreas, (each pre-soaked in PBS at a pH of 7.4 for 24 h prior). Ultrasound imaging of the initial dimensions occurred after abdominal closure. Reopening the abdomen and disrupting the respective organs initiated fluid leakage. Creation of a gastrotomy on the front gastric wall adjacent to BioSUM1 (distance between BioSUM1 and incision varied between 2 cm and 5 cm) stimulated gastric leakage.

Similarly, a 1-cm enterotomy near BioSUM2 on the small intestine (distance between BioSUM2 and incision varied between 2 cm and 5 cm) simulated intestinal leakage. Resection of the pancreas tail adjacent to BioSUM3 (distance between BioSUM3 and incision varied between 2 cm and 5 cm) initiated pancreatic leakage. Closing the abdominal wall immediately after creating these gastrointestinal leaks allowed ultrasound imaging to monitor dimensional changes of BioSUMs for 2 h. Sham experiments involved placing BioSUMs on the designated organs without subsequent disruption, with similar imaging procedures. The BioSUMs were subsequently collected to evaluate their integrity, and the animals were euthanized with pentobarbital (150 mg/kg).

### In Vitro Tests of Cytocompatibility

In vitro cytocompatibility tests involved L929 (a mouse fibroblast cell line representing connective mouse tissue) for the cell culture, CellTiter-Glo® Luminescent Cell Viability Assay (Promega, G7571) as the cell viability assay, both the Live/Dead cytotoxicity kit (Invitrogen, L3224) with BioTek Lionheart system for imaging, and Microplate reader Synergy H1 as the plate reader. BioSUM1, BioSUM2, and BioSUM3 were 2 mm in diameter and 200 μm thick with a 500 μm diameter, 25 μm thick Zn disk in the center, the dimensions of which proportionally decreased from the original values. Cytotoxic concentration of dimethyl sulfoxide (DMSO) served as positive control. Biocompatible crosslinked polyethylene glycol diacrylate (PEGDA, Mₙ 250) served as negative control. Introducing 1 wt % biocompatible photoinitiator Irgacure 2959 and subsequent UV exposure (365 nm, 500 mW/cm²) for 4 min under a nitrogen atmosphere yielded the crosslinked PEGDA Mₙ 250. L929 cells were seeded in 24-well plates at a density of 10,000 cells per well. Treatment of three types of BioSUMs prior to transfer to 24-well plates included soaking in PBS (pH 7.4) for 24 h, sterilization in 75% ethanol for 1 h, soaking in culture media for 24 h and sterilization by UV exposure for 1 h. BioSUMs were directly incubated with cells. Three individual samples were removed on designated days (day 1, day 4, and day 7) for imaging and cell viability assay.

### Evaluation of Biocompatibility

All procedures were approved by the Institutional Animal Care and Use Committee of Washington University in St. Louis protocol (protocol #22-0022). Adult male Lewis rats (8-10 weeks old) were purchased from Charles River Laboratories, Wilmington, MA. The implantation procedures of BioSUM were the same as described above. Daily monitoring of the rats confirmed their healthy conditions and stress levels. Euthanizing four rats at 2-, and 4-weeks post-implantation enabled explantation of organs, including the stomach, pancreas, and intestine, for histological evaluations. Organs were stored in 10% neutral buffered formalin in 50-ml centrifuge tubes. Staff at the Mouse Histology and Phenotyping Laboratory at Northwestern University conducted paraffin embedding, sectioning (section thickness=4 μm, 1-2 tissue cuts on each slide), and hematoxylin and eosin (H&E) staining based on standard protocols. Explantation of heart, lung, kidney, spleen, and extraction of blood from the heart with 26-g syringes at 2-, and 4-weeks post-implantation enabled elemental analysis by staff at the Quantitative Bio-element Imaging Center (QBIC) at Northwestern University. Organs and blood for elemental analysis were stored in pre-weighed 50-ml centrifuge tubes at −20° C. Subsequent dissolution of tissues by addition of 1.5 mL nitric acid and 0.35 mL hydrogen peroxide and dilution to 10 wt % allowed for ICP-MS analysis of Zn concentrations. Blood chemistry tests used blood samples collected from the heart with 26-g syringes at 2-, and 4-weeks post-implantation. Samples were collected in serum separator tubes and spun to separate serum from clotted red cells. The serum layers were transferred to another empty tube and kept at −20° C. Staff at the Veterinary Diagnostic Laboratory at the University of Illinois Urbana-Champaign conducted tests on these blood samples.

### Statistical Analysis

Required sample sizes were estimated based on previous publications and experience. The numbers of replicates are reported, and several internal replications are present in the studies. No data were excluded after analysis. Animals were randomly assigned to treatment groups. Group statistical analyses were performed using GraphPad Prism software (GraphPad, LaJolla, CA) and Origin (OriginLab, Northampton, MA). For N sizes, the number of trials and the number of animals is provided. All data are expressed as mean±SD or individual plots. For multiple group comparisons, one-way or two-way analysis of variance (ANOVA) tests were used for normally distributed data, followed by post hoc analyses reported in Figure legends. p<0.05 was considered statistically significant.

### Monitoring Homeostatic Dysregulation

BioSUMs enable detection of unpredictable leakage and assessment of different cases of homeostatic dysregulation in deep tissues. The technique relies on converting dimensional changes of a shape-adaptive matrix material into ultrasound-readable signals. Designs of BioSUMs reported here allow for the monitoring of pH changes associated with anastomotic leakage following GI surgeries, pleural effusion, ascites and complex infections. Other materials and ultrasonic indicators create opportunities for monitoring other forms of homeostatic dysregulation. An example in the context of detecting brain hemorrhage may incorporate a hemostatic agent inside the matrix material. Specific binding with red blood cells and absorption of blood will result in expansion as the basis for ultrasound detection. Alternatively, thermoresponsive materials such as those that incorporate polyethylene glycol grafted into a chitosan hydrogel may enable sensing of changes in temperature. These examples suggest broad applicability of the BioSUM concept in the assessment of diverse forms of homeostatic dysregulation in deep tissues.

Integration of BioSUMs onto Tissue Surfaces with Bioresorbable Adhesives

Bioresorbable adhesive can be used to integrate BioSUM onto tissue surfaces as an alternative approach to anchoring the device for postoperative monitoring. The integration starts with coating the tissue surfaces with a primer layer consisting of chitosan, sulfated N-hydroxysuccinimide (Sulfo-NHS) and (1-ethyl-3-(3-dimethylaminopropyl) carbodiimide (EDC), followed by applying the two-component bioresorbable adhesive onto the device and tissue surfaces. The two-component adhesive is a viscous liquid solution of a photocurable covalent network of polyethylene glycol-lactide acid diacrylate (PEG-LA-DA) and an ionic network of sodium alginate. Exposure to UV light (365 nm, 20 mW/cm²) for 3 min completes the integration of the device onto tissue surfaces with robust chemical bonding (FIG. 6).

The photo-triggered liquid-solid transformation of the bioresorbable adhesive enables the formation of bonding without applying external forces, thereby facilitating the integration procedure, and minimizing the potential damage to the tissues. The primary amine groups in chitosan form chemical bonds with negatively charged carboxylic acid groups on tissue surfaces and the sodium alginate in the adhesive. The Sulfo-NHS and EDC promote robust bonding between the adhesive and the BioSUM. All components in the adhesive undergo controllable bioresorption via natural processes. The timeframe is tunable from 20 days to several months in PBS (pH=7.4) at 37° C. depending on the molecular weight of lactide (LA) content, to align timescales that satisfy clinical requirements.

### The Severity of Anastomotic Leakage and Detection of Leakage Via BioSUMs

Anastomotic leakage is a major cause of morbidity and mortality in postoperative complications that often occur during the 2 week period after surgeries. The incidence rate ranges from 5% to 25% depending on the sites of anastomosis, posing a significant risk to patients undergoing GI surgeries. Early symptoms such as stomach pain, fever, and nausea are often indistinguishable from causes for other diseases. As such, identification of anastomotic leakage in many cases occurs too late to prevent symptoms from being severely septic and life-threatening.

In addition, no standard approaches for diagnosis currently exist in clinical practice. Clinical assessments rely on a variety of methods and criteria, including external symptoms, contrast-enhanced computed tomography (CT), laboratory biomarkers such as C-reactive protein and amylase, and endoscopy. These approaches are either high in cost, invasive, or they pose substantial risks of radiation exposure. Therefore, a remaining challenge lies in detecting anastomotic leakage accurately to enable early therapeutic interventions.

In this context, implantation of BioSUMs near the anastomosis during the surgery enables post-surgical monitoring and detection of potential anastomotic leakage in a timely and reliable manner. If no leakage occurs postoperatively, the dimensions of BioSUMs will show minimal fluctuations with a stable distance between Zn disks in ultrasound B-mode images. When leakage occurs, expansion of the BioSUMs will increase the distance between Zn disks in B-mode images as an indication of leakage. The dimensions of the BioSUMs reflect the local pH values associated with the leak, serving as a potential indicator for leakage severity. After the diagnosis, the BioSUMs resorb into the body via natural processes and metabolic reactions, eliminating the need for extra surgical interventions.

### Bioresorption of the Hydrogel Materials Used in BioSUMs

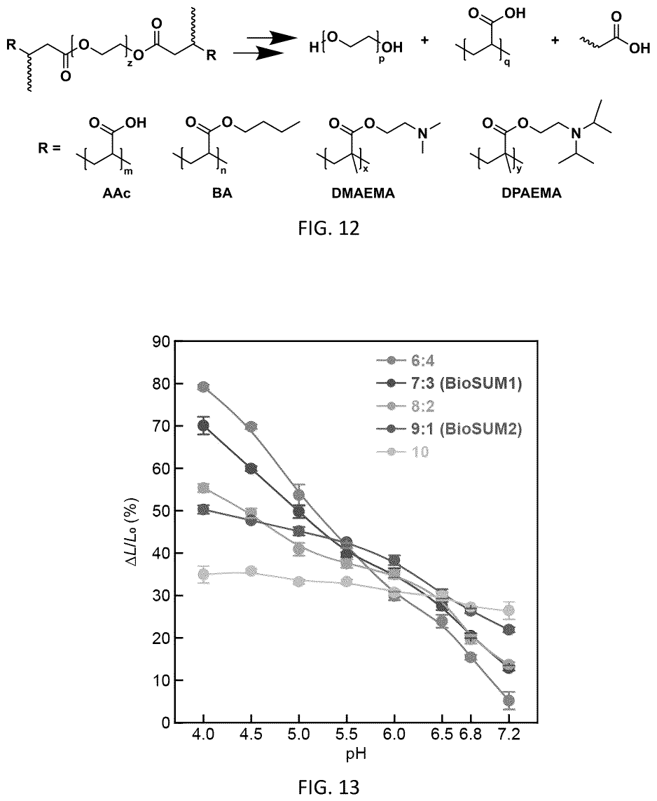

Bioresorption of the hydrogel materials used in BioSUMs is mainly attributed to hydrolytic chain scission reactions via a synergistic effect of the hydrolyzable ester linkages and the cationic or anionic nature of the pH-responsive blocks (FIG. 12). These hydrolyzable ester linkages from the PEGDA component form crosslinked hydrogel networks the enable hydrolysis of the backbone structures for all three BioSUMs. In addition, the ester linkages associated with the methacrylate of the pH-responsive blocks become susceptible to hydrolysis due to the process of protonation of tertiary amine and carboxyl functional groups that increases the hydrophilicity of polymer chains. Previous studies provide additional details about the mechanisms associated with hydrogel materials used in BioSUM1 and BioSUM2, and hydrogel materials in BioSUM3. These hydrolytic reactions lead to oligo(ethylene glycol), acrylic acid oligomers, and small molecular acids, which undergo further conversion into biocompatible and absorbable products with the assistance of enzymes and metabolic reactions.

### Materials Designs for BioSUMs

pH-responsive hydrogel materials for BioSUM1 and BioSUM2 involve p(DMAEMA-DPAEMA) copolymers with ratios of 7:3 and 9:1 between DMAEMA and DPAEMA, respectively, designed to maximize their responses in relevant pH ranges while maintaining stable mechanical properties (FIGS. 13-14, Tables 2-4). Protonation of tertiary amine moieties in both DMAEMA and DPAEMA leads to a conformation change of the hydrogels from hydrophobic to hydrophilic as the pH decreases. DMAEMA exhibits a higher pKa value (~8.4) compared to DPAEMA (~6.3). Therefore, a higher ratio of DMAEMA in the copolymer enables a larger swelling ratio for BioSUM2 near the physiological pH of 7.4, appropriate for the relevant range of pH values in the small intestinal environment (pH~6.8). A higher ratio of DPAEMA in the copolymer results in a larger Young's modulus and elongation at break in BioSUM1 (Table 4), allowing stable operation in the strongly acidic environment.

The hydrogel material used in BioSUM3 contains pH-responsive monomer AAc and crosslinker PEGDA. The introduction of hydrophobic BA shifts the pKa value beyond the physiological pH of 7.4. Similar to the designs for BioSUM1 and BioSUM2, tuning the AAc ratio and the degree of crosslinking aim to balance between the response of BioSUM3 in alkaline conditions and its mechanical properties (FIGS. 14-15, Tables 2-3 and 5).

Selecting bioresorbable metals (Mg, Fe, Zn, Mo, W) as ultrasonic indicators takes into consideration their degradation rates and acoustic impedance mismatch with the surrounding hydrogel and soft tissues. Numerical simulation results in FIG. 16 indicate similar SNR values for each of these metals. A comparison between their degradation rates in physiological conditions leads to the choice of Zn as ultrasonic indicators due to its capability to dissolve in several months.

### The Theoretical Model and Finite Element Analysis Approach for the Swelling of Hydrogels

Chain extensions of pH-responsive monomers DMAEMA and DPAEMA for BioSUM1 and BioSUM2, and hydrophobic BA monomer for BioSUM3 into oligomers prior to crosslinking form grafted side chains in the hydrogel networks. These side chains together with physical entanglements attributed to the low crosslinking density provide BioSUM with considerable equilibrium swelling ratios within a timeframe of 2 h.

In the theoretical model, the force balance equation and mass conservation of the water govern the evolution of the gel. The force balance equation is

$$div σ = 0 \qquad ( 1 )$$

where α is the Cauchy stress of the gel's network and it is expressed as

$$σ = \frac{NkT}{J} ( {FF}^{T} - I ) - Π I \qquad ( 2 )$$

where I is the second-order identity tensor, F is the deformation gradient with respect to the dry polymer state, J=det F is the volume swelling ratio of the gel, Π is the osmotic pressure, Nis the number of chains per reference volume in the network, k is the Boltzmann constant and T is the absolute temperature.

The mass conservation equation of the water is

$$\frac{{dC}_{m}}{dt} + J div {j}_{m} = 0 \qquad ( 3 )$$

where Cₘ is the number of water molecules per volume of dry polymer and jₘ is the flux of the water,

$${j}_{m} = - \frac{D}{k T} \frac{{C}_{m}}{J} g r a d {μ}_{m} \qquad ( 4 )$$

where D is the diffusivity, and μₘ is the chemical potential of the water, which is expressed as

$${μ}_{m} = k T ( ln \frac{J - 1}{J} + \frac{1}{J} + \frac{χ}{{J}^{2}} ) + Π v \qquad ( 5 )$$

where χ is the Flory-Huggins parameters and v is the volume of one water molecule. The parameter χ characterizes the hydrophilicity of the polymer network, thus governing the swelling ratio of the gel. Consequently, immersion of the gel in solutions with different pH values leads to variations in the quantity of fixed charges present within the network, yielding different X values and resulting in different equilibrium swelling ratios.

A second-order continuous step function of the swelling ratio J models the diffusivity. This representation, implemented within COMSOL 6.0, effectively captures the phenomenon wherein diffusivity remains limited when the swelling ratio is small. Four parameters, namely Dₗ, Dₕ, J_{mid}, and J_{range}, collectively determine the relationship of D with J (FIG. 17).

The incompressible condition postulates that both the polymer molecules and the water molecules are incompressible. Thus, any alteration in the volume of the gel is solely ascribed to the change in the water concentration:

$$J = 1 + v {C}_{m} \qquad ( 6 )$$

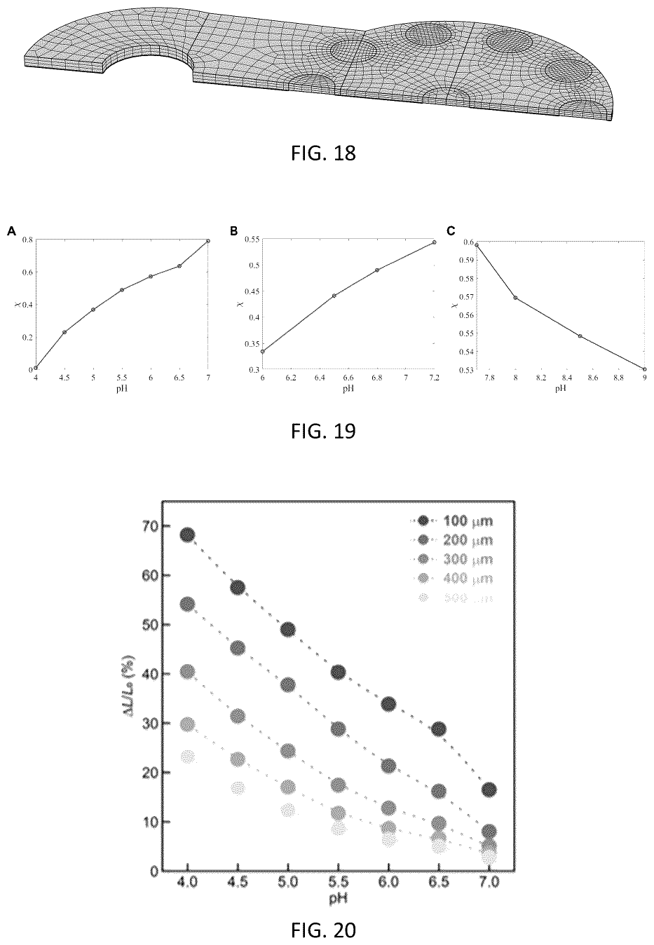

COMSOL 6.0 defines the finite element model and FIG. 18 demonstrates the set up. The geometry implemented in the model replicates the experimental setup, with one-quarter of the device modeled to account for inherent symmetry. In the first step, we fit X at different pH levels by gel's equilibrium swelling ratio as measured by experiments (FIG. 19). The results are within a reasonable range and are close to the available literature values. Nv values of the sample are obtained from the experimental recipe; they are 0.055, 0.039 and 0.0068 for BioSUM1, BioSUM2, and BioSUM3, respectively. In the second step, the gel undergoes swelling from its initial state. Experimental results determine the initial swelling ratio. At time t=0, the chemical potential, μₘ, at the external surface is set as 0, thereby facilitating the swelling process. Fitting the swelling curves across all pH conditions involves the proper selection of diffusivity constants. Table 1 presents the fitted parameters. Proper diffusivity constants are selected to fit the swelling curve across all pH conditions. The fitted parameters are listed in Table 1 and they are close to literature values.

### The Theoretical Model for the Effect of Metal Thickness on the Reflection of Acoustic Waves

A theoretical model in one-dimension was derived to study the effect of metal thickness on the reflection at the metal/tissue interface. A metal layer of thickness h_{Metal} was embedded in infinitely large tissues (FIG. 19). In the one-dimensional case, the acoustic wave velocity in tissue c_{Tissue} is related to the modulus of tissue E_{Tissue} and density ρ_{Tissue} via

$${c}_{Tissue} = \sqrt{\frac{{E}_{Tissue}}{{ρ}_{Tissue}}} .$$

Similarly, the acoustic wave velocity in metal is

$${c}_{Metal} = \sqrt{\frac{{E}_{Metal}}{{ρ}_{Metal}}} ,$$

with E_{Metal} and density ρ_{Metal} representing the modulus of metal and the density of metal, respectively. For a harmonic wave with frequency f (angular frequency ω=2πf), waves in the tissue above the metal (z<0, panel A of FIG. 19) are the sum of the incident wave (Uₗ) and the reflected wave (U_{R}), such that the displacement can be expressed in complex form as

$${u}_{1} = {U}_{I} + {U}_{R} = {A}_{I} exp [ i ( ω t + {k}_{Tissue} z ) ] + {A}_{R} exp [ i ( ω t - {k}_{Tissue} z ) ] , \qquad ( 7 )$$

where

$${k}_{Tissue} = \frac{ω}{{c}_{Tissue}}$$

is the wavenumber, and i is the symbol for the complex number. The wave in the metal was the sum of the transmitted wave (U_{T}) at the metal/tissue interface at z=0 and the reflected wave (U_{TR}) at the metal/tissue interface at z=h_{Metal}, such that the displacement in the metal is

$${u}_{2} = {U}_{T} + {U}_{T R} = {A}_{T} exp [ i ( ω t + {k}_{Metal} z ) ] + {A}_{T R} exp [ i ( ω t - {k}_{Metal} z ) ] . \qquad ( 8 )$$

The wave in the tissue below the metal is the transmitted wave (U_{TT}) at the metal/tissue interface at z=h_{Metal}, such that the displacement in the tissue below metal is

$${u}_{3} = {U}_{T T} = {A}_{T T} exp [ i ( ω t + {k}_{Tissue} z ) ] . \qquad ( 9 )$$

The continuity of displacements and stresses at the metal/tissue interfaces requires that

$${u}_{1} = {u}_{2} and {E}_{Tissue} \frac{\partial {u}_{1}}{\partial z} = {E}_{Metal} \frac{\partial {u}_{2}}{\partial z} at z = 0 , \qquad ( 10 )$$

$$and$$

$${u}_{2} = {u}_{3} and {E}_{Metal} \frac{\partial {u}_{2}}{\partial z} = {E}_{Tissue} \frac{\partial {u}_{3}}{\partial z} at z = {z}_{Metal} . \qquad ( 11 )$$

The ratio of the amplitude of the reflected wave over that of the incident wave at the metal/tissue interface at z=0 can be solved as

$$( 12 )$$

$$\vert \frac{{A}_{R}}{{A}_{I}} \vert = \frac{2 {A}_{R} ( {z}_{Metal}^{2} - {z}_{Tissue}^{2} ) sin ( \frac{2 π {h}_{Metal}}{{λ}_{Metal}} )}{\sqrt{{( {z}_{Metal} - {z}_{Tissue} )}^{4} + {( {z}_{Metal} + {z}_{Tissue} )}^{4} - 2 {( {z}_{Metal}^{2} - {z}_{Tissue}^{2} )}^{2} cos ( \frac{4 π {h}_{Metal}}{{λ}_{Metal}} )}} ,$$

where z_{Metal} and z_{Tissue} are the acoustic impedance of metal and tissue, respectively, and

$${λ}_{Metal} = \frac{{c}_{Metal}}{f}$$

is the wavelength in metal. Equation 12 suggests that when the metal thickness h_{Metal} is much smaller than the wavelength in metal λ_{Metal}, the amplitude of the reflected wave increases with h_{Metal}/λ_{Metal}. For the metal used in the current work (Zn) and a typical frequency of f=5 MHz, λ_{Metal}≈560 μm, and the relationship of

$$\vert \frac{{A}_{R}}{{A}_{I}} \vert vs . \frac{{h}_{Metal}}{{λ}_{Metal}}$$

is shown in panel B of FIG. 19. The increase of

$$\vert \frac{{A}_{R}}{{A}_{I}} \vert$$

almost saturates at h_{Metal}/λ_{Metal}=0.05, which justifies the use of 25 μm thick Zn disks in this work.

### Expansion of a BioSUM and Ultrasound Measurement in a Curvilinear Tissue Environment

When a BioSUM is in the curvilinear tissue environment, the expansion is isotropic with minimal deviation of measurements between each pair of Zn disks (panels A-C of FIG. 33). FEA modeling results (panels D-F of FIG. 33) demonstrate less than 2% deviation between measurements at different circular angles under various swelling ratios. In the modeling, the device was placed on a rigid cylinder with a diameter of 2.5 cm to simulate the curvilinear condition, comparable to that associated with the average diameter of the small intestine.

In the case where the BioSUM lies on a curved surface of the GI organs, the signal from three dots along the diameter appears bent in the ultrasound B-mode image, with an actual length of the arc x₀ (panel A of FIG. 34). The sum of two straight lines from the left to the middle xi and the middle to the right x₂ form the measurement length x in ultrasonic measurement (panel B of FIG. 34). Considering the maximum deviation of ultrasonic measurement Δx/x₀~0.1 at the depth of 15 cm (panel C of FIG. 34),

$$\frac{x}{{x}_{0}} = \frac{{x}_{2}}{\frac{{x}_{0}}{2}} = \frac{2 r sin \frac{θ}{2}}{r θ} = \frac{sin \frac{θ}{2}}{\frac{θ}{2}} = 0.9 \qquad ( 13 )$$

$$θ = 90 ° \qquad ( 14 )$$

This result indicates that a bending curvature less than that of a semi-sphere is sufficient to provide an ultrasonic measurement with deviation smaller than 0.1, which is the case for GI organs.

### Discussion on Mechanical Deformations

In clinical scenarios, BioSUM deployed in the GI tract may exhibit artifacts due to mechanical deformations of the stomach due to processes of consuming and digesting food. The presence of interstitial fluid between the device and the underlying tissue, and a single anchoring site (panel A of FIG. 44) lead to minimal effects of these processes on the dimensions of the device. Experimental and FEA modeling results (panel B of FIG. 44) show negligible changes of the measured distances between pairs of Zn disks. The experiments (panel C of FIG. 44) involve PBS solution between the device and an elastomeric substrate, to simulate the interstitial fluid and the adjacent soft tissue. Stretching the elastomeric substrate to ~60% mimics the mechanical deformation of the stomach before and after consuming food. Measurements of the distances between pairs of Zn disks before and after stretching the elastomer yield results in panel B of FIG. 44. FEA modeling approximates the shape of the stomach as a sphere, and takes the volume of the stomach in the fasting state, and fed state as 200 mL and 800 mL, with a continuous isotropic expansion over a period of 3 h. The expansion of the sphere imposes a tensile force on the device via shear through a thin layer of fluid. The results show that the shear pressure applied to the bottom surface of the device is 1.16×10⁻⁵ Pa. The resulting changes of displacement U of the device appear negligible (panel D of FIG. 44).

In sum, this invention discloses, among other things, bioresorbable, shape-adaptive structures that enable rapid, noninvasive measurements of homeostasis in deep tissues by conventional ultrasound imaging techniques. The swelling of thin films of a responsive hydrogel matrix induced by homeostatic perturbations leads to changes in separations between sparse collections of bioresorbable metal elements, as indicators whose positions can be determined accurately by ultrasound. The large mismatch between the acoustic impedance of these elements and the surrounding materials produces high contrast in ultrasound images, thereby allowing for accurate measurements of their separations, and thus local physical or chemical characteristics of the surrounding tissues in shallow or deep locations. An envisioned clinical scenario is in real-time detection of anastomotic leakage via changes in pH during a period of recovery after a GI surgery, to allow for early intervention. The devices survive for a relevant timeframe and then naturally bioresorb to eliminate the need for secondary surgical extraction procedures. In vivo demonstrations of this concept in small and large animals validate materials designs tailored for use in gastric, small intestinal, and pancreatic leakage.

### Example 3

### Ultrasound Sensors

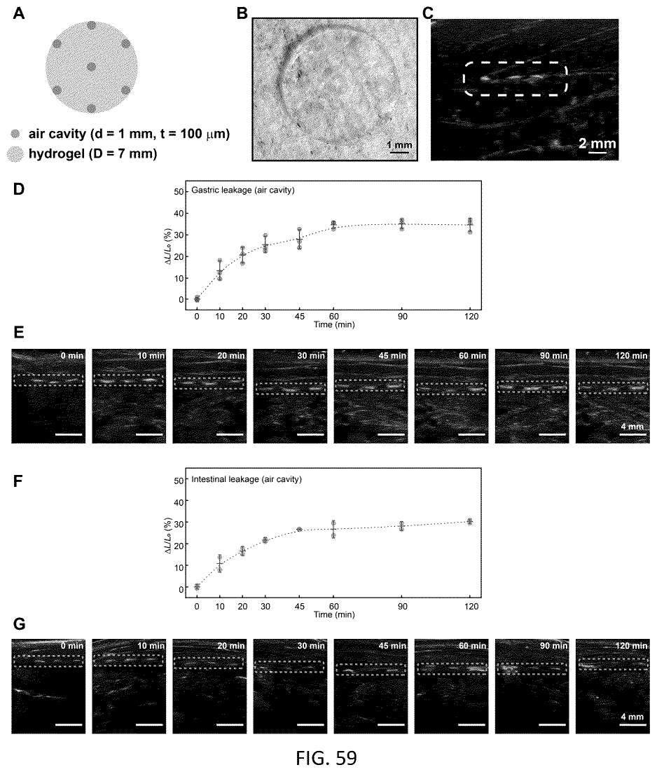

Panel A of FIG. 59 is the schematic illustration of design, where the hydrogel sensor has a diameter of 7 mm, and the embedded air cavities have a diameter of 1 mm with a height of 500 μm. Panel B of FIG. 59 is the top view optical image of a sensor with air cavities. When placed underneath a pork phantom of 1 cm depth, the sensor appears as three equally distributed bright segments in the ultrasound B-mode image (Panel C of FIG. 59), indicating the large acoustic impedance mismatch between air and surrounding hydrogels and tissues. Panels D-G of FIG. 59 demonstrate the expansion of the sensors with air cavities following gastric leakage and small intestinal leakage in a rat model, respectively.

The fabrication of air cavities began with the synthesis of hydrophobic encapsulation material. 3D molds drawn by AutoCAD with specific parameters define the geometry of the air cavity. Pouring the precursor solution of the encapsulation material into the 3D printed mold and subsequent UV crosslinking forms the bottom part of the air cavity. Sealing the air cavity with the same precursor solution completes the fabrication of the air cavity.

Fabrication of the hydrogel sensor with air cavities began with blade coating of hydrogel precursor and partial curing into a 150 μm thick film by UV exposure under a nitrogen atmosphere on a glass slide treated with 0.2 vol % trichloro(octadecyl)silane hexane solution to ensure easy detachment after fabrication. Previously fabricated air cavities serve as ultrasonic indicators. Assembling the air cavities into the designated symmetric pattern onto the partially cured hydrogel film and subsequently blade coating an additional 150 μm thick hydrogel layer enclosed the air cavities in the middle. Punching the hydrogel into desired disks using a hollow hole punch with preset diameter completes the fabrication of the hydrogel sensors with air cavities.

### Example 4

### MRI Sensors

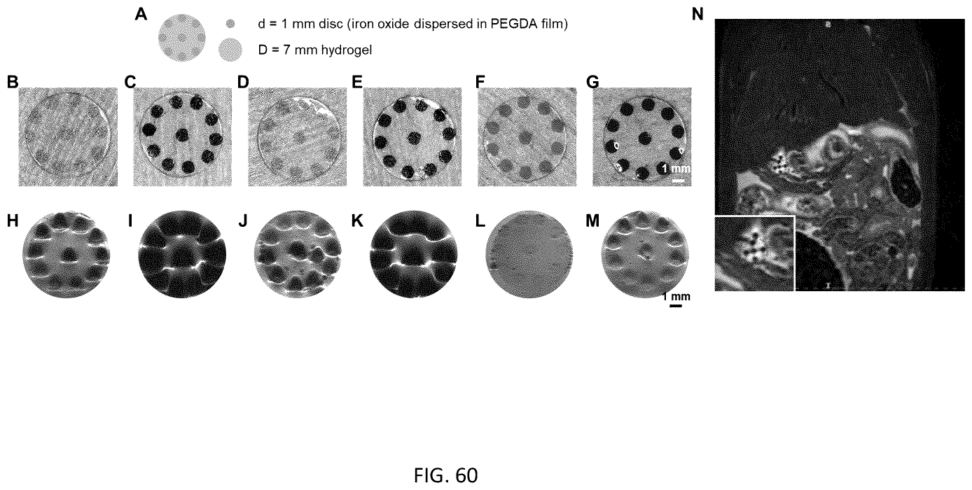

Panel A of FIG. 60 is the schematic illustration of design. Iron oxide nanoparticles or microparticles dispersed inside the polymeric poly(ethylene glycol) diacrylate (PEGDA) film serve as MRI contrast agent. The hydrogel sensor has a diameter of 7 mm. Panels B-G of FIG. 60 are optical images of sensors with different types and concentrations of iron oxide particles. Panels H-M of FIG. 60 are corresponding T2 weighted images of sensors after equilibrium in PBS solution by MRI. Both 1 wt % Fe₃O₄ nanoparticle and microparticle distort the local magnetic field too much, so the dimension of iron oxide discs is larger than their actual sizes in T2 weighted images, while 1 wt % of Fe₂O₃ microparticle is not sufficient for contrast generation. By introducing 10 wt % Fe₂O₃ microparticle inside PEGDA for contrast generation, the measurement length of iron oxide discs shows a deviation of approximately 500 μm when implanting the sensor on the stomach in a rat (Panel N of FIG. 60). The fabrication procedure of MRI sensors is similar to that of the ultrasound sensors with air cavities.

### Example 5

### CT Sensors

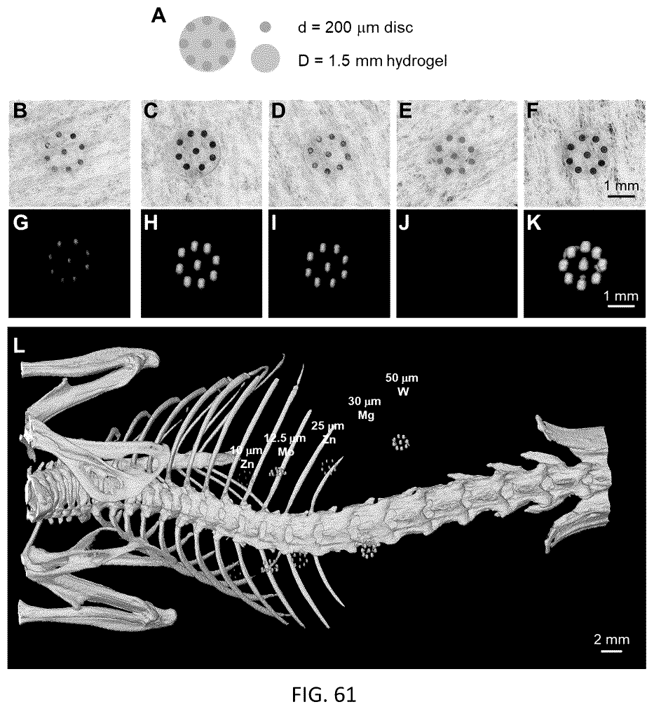

Panel A of FIG. 61 is the schematic illustration of design. Different types and thickness of metal discs serve as CT contrast agent. The hydrogel sensor has a diameter of 1.5 mm. Panels B-F of FIG. 61 are optical images of sensors. Panels G-K of FIG. 61 are corresponding CT images of sensors in air. CT contrast depends on x-ray mass attenuation coefficients and the thickness of metals, but thicker metals result in stronger scatter and the increased size of metal dots, which in turn affects the resolution and measurement. Measurements by imageJ indicates that 10 μm Zn, 12.5 μm Mo or 25 μm Zn have both sufficient contrast and resolution in CT (Panels G-K of FIG. 61). The CT image of sensors in a mouse (Panel L of FIG. 61) shows similar results. The fabrication procedure of CT sensors is similar to that of the ultrasound sensors with air cavities.

### Example 6

### Multimodal Imaging

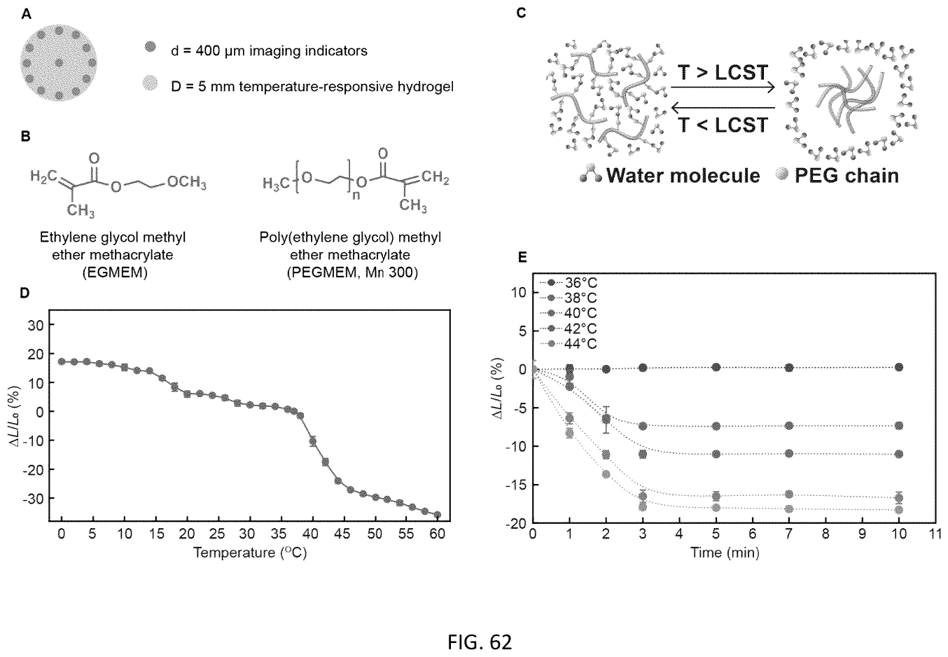

Panel A of FIG. 62 is the schematic illustration of design, where the hydrogel sensor has a diameter of 5 mm, and the embedded imaging indicators have a diameter of 400 μm with a height of 25 μₘ. Panel B of FIG. 62 is the chemical structures of the monomers, oligomers in the temperature-responsive hydrogels. The temperature responses of the hydrogel originate from its chemical compositions. The sensor is based on a copolymer of EGMEM and PEGMEM, using poly(ethylene glycol) diacrylate (PEGDA) as cross-linker. Panel C of FIG. 62 is the schematic illustration of the mechanism for temperature-responsive hydrogels governed by the lower critical solution temperature (LCST). By fine-tuning the monomer ratios and adjusting the oligomerization time, we are able to tune the LCST to fall within the 37-44° C. range. When the temperature rises above the LCST, polymer-water interactions weaken, the polymer chains become less hydrated and aggregate, and the hydrogel deswells. Below the LCST, the PEG-containing network remains highly hydrated due to favorable hydrogen bonding, resulting in a swollen state. Panel D of FIG. 62 is the thermally induced dimensional change of the temperature-responsive hydrogel. The experimental measurements of the swelling ratios show the temperature-dependent swelling behavior of hydrogel sensor at different temperature values, in terms of the percentage change in length (ΔL/L₀%). As the temperature increases, ΔL/L₀% decreases and exhibits a pronounced drop around ~35-45° C. Panel E of FIG. 62 is the time-dependent responses of temperature-responsive hydrogels upon immersion in solutions at different temperatures (36-44° C.). The hydrogel sensor exhibits around 20% volumetric change within the target temperature range, achieving the sensitivity required for applications under physiological conditions.

The fabrication of imaging indicators was performed by laser cutting. The pattern drawn in AutoCAD with specified parameters defines the geometry of the indicators.

Fabrication of the hydrogel sensor with indicators began with blade coating of hydrogel precursor and partial curing into a 150 μm thick film by UV exposure under a nitrogen atmosphere on a glass slide treated with 0.2 vol % trichloro(octadecyl)silane hexane solution to ensure easy detachment after fabrication. Previously fabricated Zn and iron oxide discs served as ultrasonic indicators. Assembling the indicators into the designated symmetric pattern onto the partially cured hydrogel film and subsequently blade coating an additional 150 μm thick hydrogel layer enclosed the indicators in the middle. Punching the hydrogel into desired disks using a hollow hole punch with preset diameter completes the fabrication of the hydrogel sensors with indicators.

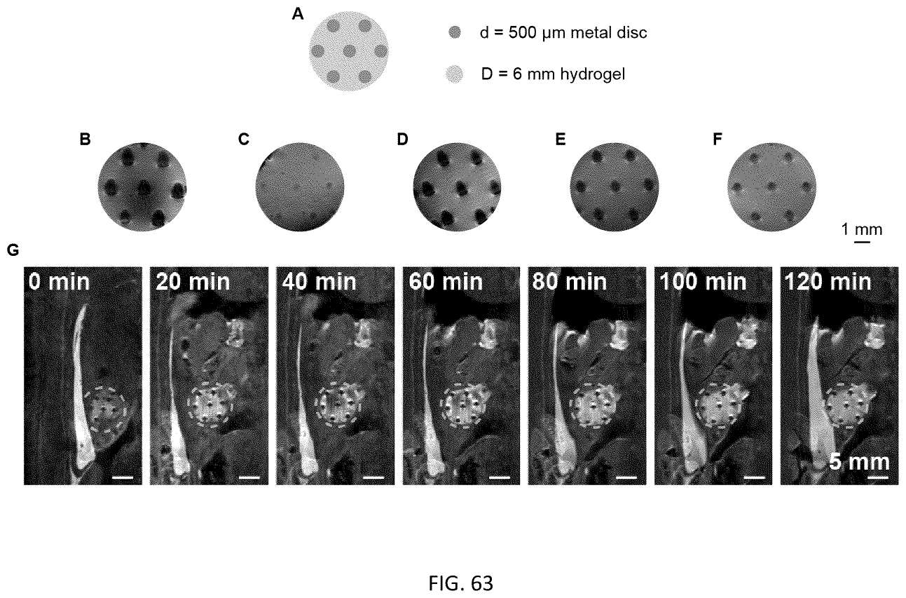

Panel A of FIG. 63 is the schematic illustration of design. Different types of metal discs with the diameter of 500 μm and thickness of 25 μm serve as MRI contrast agents. The hydrogel sensor has a diameter of 6 mm. Panels V-F of FIG. 63 are MRI images of sensors with different types of metal discs. By incorporating 20 wt % Fe₂O₃ microparticles into the PEGDA matrix to enhance T2 contrast, we were able to track the sensor geometry over time after acute gastrotomy in rat model. Over the 2-hour period, the sensor gradually expanded and reached equilibrium, as evidenced by the increasing spacing between the Fe₂O₃ disks on T2-weighted MRI images. (Panel G of FIG. 63) The fabrication procedure of pH-responsive MRI sensors is similar to that of the temperature-responsive ultrasound sensors.

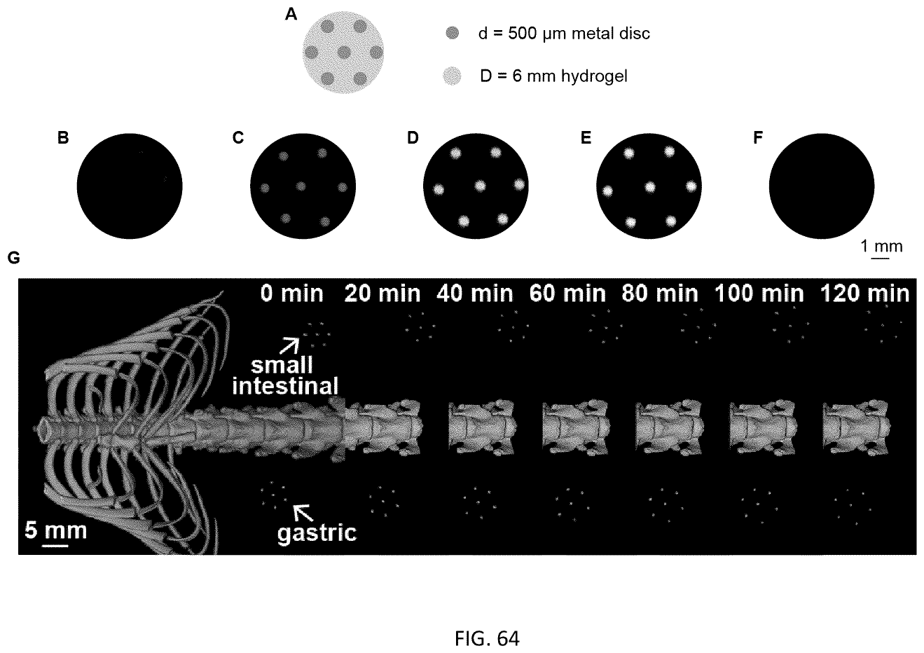

Panel A of FIG. 64 is the schematic illustration of pH-responsive CT senser design. Different types of metal (Fe₂O₃, Zn, Mo, W, and Mg) discs with the diameter of 500 μm and thickness of 25 μm serve as CT contrast agents. The hydrogel sensor has a diameter of 6 mm. Panels B-F of FIG. 64 are CT images of sensors embedded in Super-Flex, a tissue-mimicking phantom matrix. CT contrast depends on x-ray mass attenuation coefficients, which increases with effective atomic number (Z_{eff}) and density. Measurements by imageJ indicates that 25 μₘ Zn, Mo and W have both sufficient contrast and resolution in CT (FIGS. 3B-F). Zn was selected as the CT sensor due to its sufficient CT contrast and favorable degradability. By incorporating Zn discs as CT contrast agents, we were able to track the sensor geometry after acute gastrotomy in a rat model. Over the 2-hour period, the sensor gradually expanded and reached equilibrium, as evidenced by the increased spacing between the Zn discs on serial CT images (Panel G of FIG. 64). The fabrication procedure of pH-responsive CT sensors is similar to that of the temperature-responsive ultrasound sensors.

### Example 7

### Materials for Ultrasound Hydrogel Tags

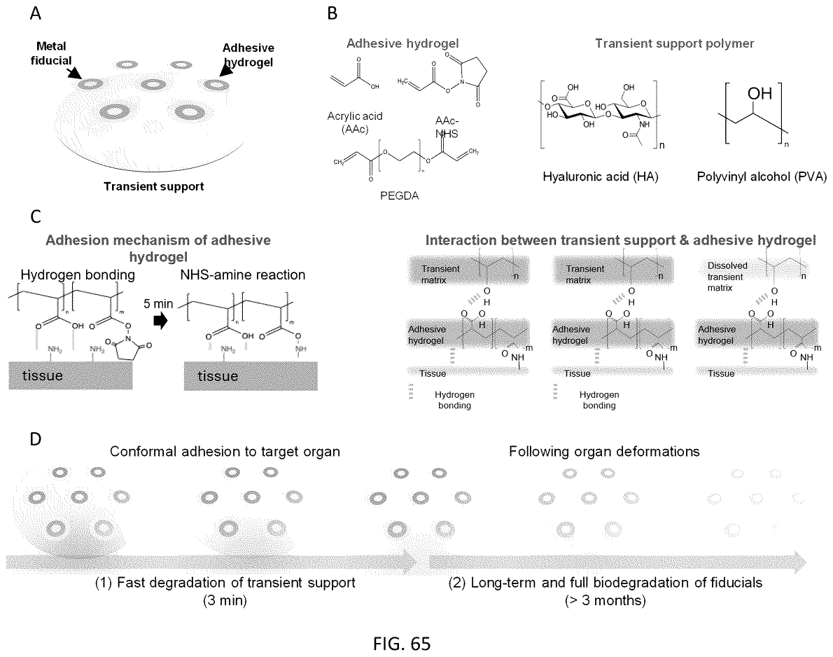

FIG. 65 shows materials for ultrasound hydrogel tags. Panel A: Adhesive hydrogel tag arrays embedded bioresorbable metals (Mo) are bonded to the transient support via hydrogen bonding. Panel B: Adhesive hydrogel with NHS functional groups is chemically bonded to amine groups of target tissue via amine-NHS reaction. Panel C: The transient matrix supports transfer of tag arrays to target organ tissue, by maintaining the structure of arrays. Panel D: Transient support matrix is quickly dissolved into biofluid within 3-5 min by biofluids. Biopolymers including hyaluronic acid and synthetic polymers including PVA which is fast dissolution rate in water/biofluids can be used for the transient support.

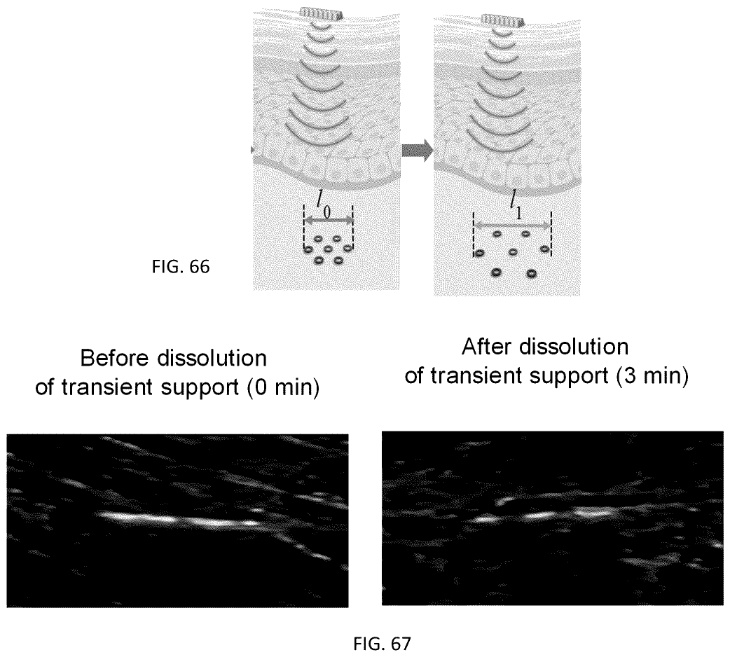

FIG. 66 shows after dissolution of transient support, displacements of tag arrays (Δl=l₁−l₀) measured to monitor mechanical deformations of the organ by ultrasound images.

FIG. 67 shows the ultrasound images before and after dissolution of the transient support.

### Example 8

### Characterization of Ultrasound Hydrogel Tags

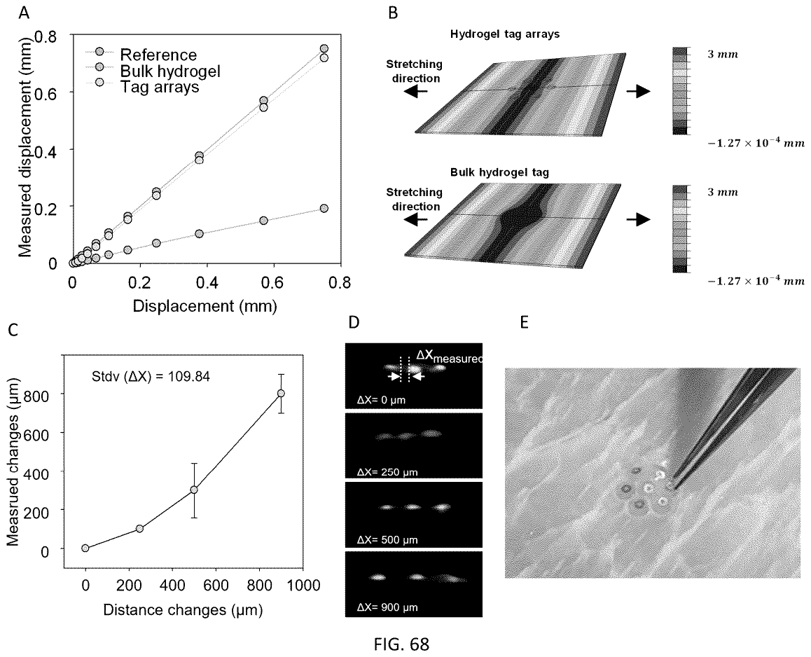

FIG. 68 shows characterization of ultrasound hydrogel tags. Panel A: Tag arrays exhibit lower errors in measuring displacements Δl than the bulk hydrogel systems. This is because of elastic modulus mismatch between tissue and hydrogel. Panel B: Finite element simulation (FEM) results show that mechanical deformations of each hydrogel system on the bladder tissue (10 kPa). Hydrogel tag arrays follow strain of the stretched bladder tissue, enabling minimizing measurement errors. By contrast, bulk hydrogel (100 kPa and thickness of 100 m) shows no significant changes in displacements on the bladder tissue, due to the elastic mismatch. Panels C-D: Ultrasound images clearly show displacements changes of each array. Panel E: Tag arrays on the pork tissue show stable adhesion, enabling follow mechanical deformations of port tissue.

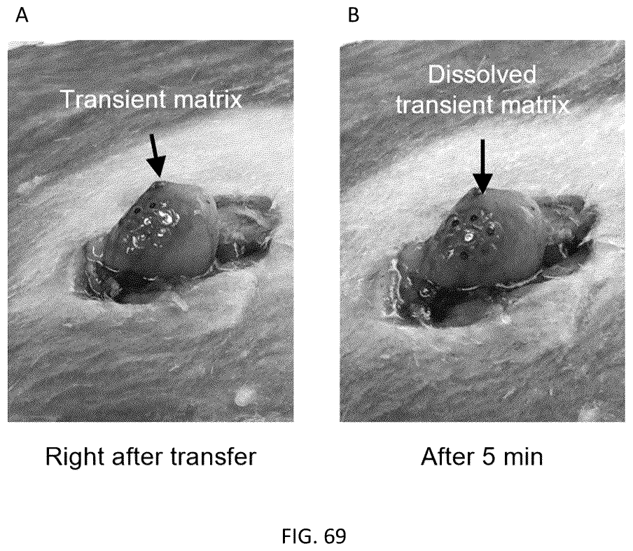

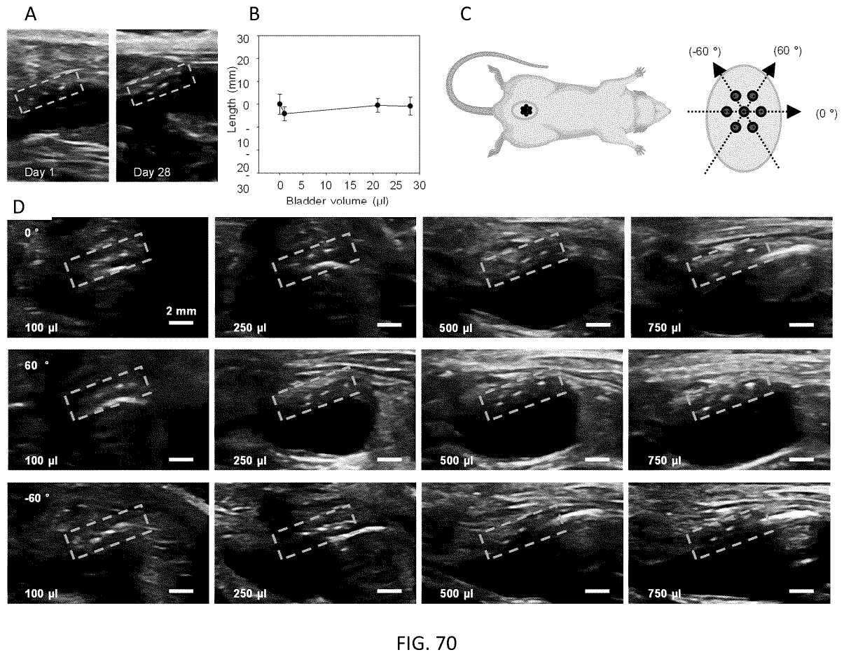

FIG. 69 shows the hydrogel tag arrays integration on tissues (here, bladder surface) without surgical stitches (Pabel A), and after transfer of a hydrogel tag array system, a transient support matrix is quickly dissolved by biofluids, remaining only tag arrays (Panel B).

### Example 9

### In Vivo Validation of Ultrasound Hydrogel Tag Arrays

FIG. 70 shows in vivo validation of ultrasound hydrogel tag arrays. Panels A-B: Tag arrays transferred on the bladder shows stable adhesion property without significant changes in displacements between tag arrays over 4 weeks in vivo. Panel C: Tag arrays designed symmetric structures with repeated angle of 60° enables local strain mapping with its specific angular directions (−60°, 0°, 60°) of tissues. Panel D: Local strain with different angular directions was measured at different volumes of infused saline into the bladder.

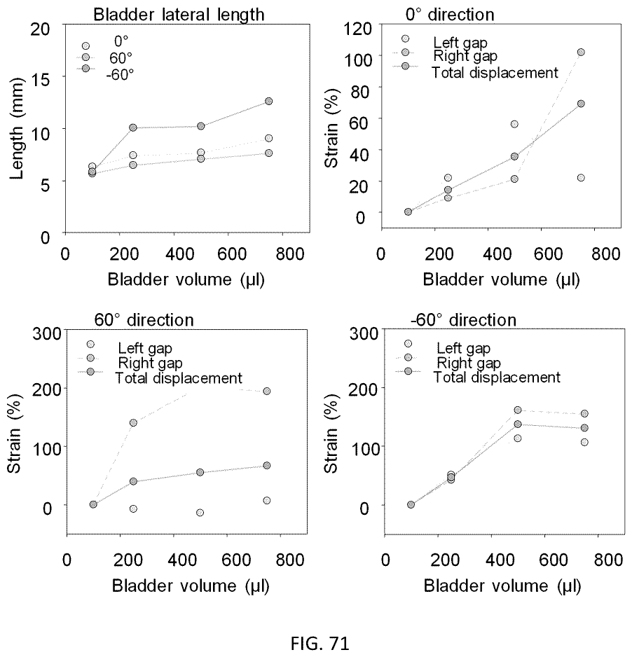

FIG. 71 shows experimental measurements of local strain showing its asymmetric expansion of bladder as a function of the volume of infused saline into the bladder. However, the measured total displacement in each direction matches the lateral expansion of the bladder. The hydrogel tag arrays enable non-invasive monitoring capabilities of mechanical organ deformation, providing spatial strain information of the tissue.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference.

### LIST OF REFERENCES

[1]. K. Kwon, J. U. Kim, S. M. Won, J. Zhao, R. Avila, H. Wang, K. S. Chun, H. Jang, K. H. Lee, J.-H. Kim, S. Yoo, Y. J. Kang, J. Kim, J. Lim, Y. Park, W. Lu, T. Kim, A. Banks, Y. Huang, J. A. Rogers, A battery-less wireless implant for the continuous monitoring of vascular pressure, flow rate and temperature. Nat. Biomed. Eng, 1-14 (2023).
[2]. C. M. Boutry, L. Beker, Y. Kaizawa, C. Vassos, H. Tran, A. C. Hinckley, R. Pfattner, S. Niu, J. Li, J. Claverie, Z. Wang, J. Chang, P. M. Fox, Z. Bao, Biodegradable and flexible arterial-pulse sensor for the wireless monitoring of blood flow. Nat Biomed Eng 3, 47-57 (2019).
[3]. M. Corsi, A. Paghi, S. Mariani, G. Golinelli, A. Debrassi, G. Egri, G. Leo, E. Vandini, A. Vilella, L. Dahne, D. Giuliani, G. Barillaro, Bioresorbable Nanostructured Chemical Sensor for Monitoring of pH Level In Vivo. Advanced Science 9, 2202062 (2022).
[4]. L. Lipani, B. G. R. Dupont, F. Doungmene, F. Marken, R. M. Tyrrell, R. H. Guy, A. Ilie, Non-invasive, transdermal, path-selective and specific glucose monitoring via a graphene-based platform. Nature Nanotech 13, 504-511 (2018).
[5]. Y. Chen, S. Lu, S. Zhang, Y. Li, Z. Qu, Y. Chen, B. Lu, X. Wang, X. Feng, Skin-like biosensor system via electrochemical channels for noninvasive blood glucose monitoring. Science Advances 3, e1701629 (2017).
[6]. H. Zhang, P. Gutruf, K. Meacham, M. C. Montana, X. Zhao, A. M. Chiarelli, A. Vazquez-Guardado, A. Norris, L. Lu, Q. Guo, C. Xu, Y. Wu, H. Zhao, X. Ning, W. Bai, I. Kandela, C. R. Haney, D. Chanda, R. W. Gereau, J. A. Rogers, Wireless, battery-free optoelectronic systems as subdermal implants for local tissue oximetry. Science Advances 5, eaaw0873 (2019).
[7]. J. Shin, Z. Liu, W. Bai, Y. Liu, Y. Yan, Y. Xue, I. Kandela, M. Pezhouh, M. R. MacEwan, Y. Huang, W. Z. Ray, W. Zhou, J. A. Rogers, Bioresorbable optical sensor systems for monitoring of intracranial pressure and temperature. Science Advances 5, eaaw1899 (2019).
[8]. L. Lu, P. Gutruf, L. Xia, D. L. Bhatti, X. Wang, A. Vazquez-Guardado, X. Ning, X. Shen, T. Sang, R. Ma, G. Pakeltis, G. Sobczak, H. Zhang, D. Seo, M. Xue, L. Yin, D. Chanda, X. Sheng, M. R. Bruchas, J. A. Rogers, Wireless optoelectronic photometers for monitoring neuronal dynamics in the deep brain. Proceedings of the National Academy of Sciences 115, E1374-E1383 (2018).
[9]. W. Bai, J. Shin, R. Fu, I. Kandela, D. Lu, X. Ni, Y. Park, Z. Liu, T. Hang, D. Wu, Y. Liu, C. R. Haney, I. Stepien, Q. Yang, J. Zhao, K. R. Nandoliya, H. Zhang, X. Sheng, L. Yin, K. MacRenaris, A. Brikha, F. Aird, M. Pezhouh, J. Hornick, W. Zhou, J. A. Rogers, Bioresorbable photonic devices for the spectroscopic characterization of physiological status and neural activity. Nat Biomed Eng 3, 644-654 (2019).
[10]. J. Kim, G. A. Salvatore, H. Araki, A. M. Chiarelli, Z. Xie, A. Banks, X. Sheng, Y. Liu, J. W. Lee, K.-I. Jang, S. Y. Heo, K. Cho, H. Luo, B. Zimmerman, J. Kim, L. Yan, X. Feng, S. Xu, M. Fabiani, G. Gratton, Y. Huang, U. Paik, J. A. Rogers, Battery-free, stretchable optoelectronic systems for wireless optical characterization of the skin. Science Advances 2, e1600418 (2016).
[11]. K. Kwon, J. U. Kim, Y. Deng, S. R. Krishnan, J. Choi, H. Jang, K. Lee, C.-J. Su, I. Yoo, Y. Wu, L. Lipschultz, J.-H. Kim, T. S. Chung, D. Wu, Y. Park, T. Kim, R. Ghaffari, S. Lee, Y. Huang, J. A. Rogers, An on-skin platform for wireless monitoring of flow rate, cumulative loss and temperature of sweat in real time. Nat Electron 4, 302-312 (2021).
[12]. R. C. Webb, Y. Ma, S. Krishnan, Y. Li, S. Yoon, X. Guo, X. Feng, Y. Shi, M. Seidel, N. H. Cho, J. Kurniawan, J. Ahad, N. Sheth, J. Kim, J. G. Taylor V I, T. Darlington, K. Chang, W. Huang, J. Ayers, A. Gruebele, R. M. Pielak, M. J. Slepian, Y. Huang, A. M. Gorbach, J. A. Rogers, Epidermal devices for noninvasive, precise, and continuous mapping of macrovascular and microvascular blood flow. Science Advances 1, e1500701 (2015).
[13]. K. Kwon, H. Wang, J. Lim, K. S. Chun, H. Jang, I. Yoo, D. Wu, A. J. Chen, C. G. Gu, L. Lipschultz, J. U. Kim, J. Kim, H. Jeong, H. Luan, Y. Park, C.-J. Su, Y. Ishida, S. R. Madhvapathy, A. Ikoma, J. W. Kwak, D. S. Yang, A. Banks, S. Xu, Y. Huang, J.-K. Chang, J. A. Rogers, Wireless, soft electronics for rapid, multisensor measurements of hydration levels in healthy and diseased skin. Proceedings of the National Academy of Sciences 118, e2020398118 (2021).
[14]. S.-W. Hwang, H. Tao, D.-H. Kim, H. Cheng, J.-K. Song, E. Rill, M. A. Brenckle, B. Panilaitis, S. M. Won, Y.-S. Kim, Y. M. Song, K. J. Yu, A. Ameen, R. Li, Y. Su, M. Yang, D. L. Kaplan, M. R. Zakin, M. J. Slepian, Y. Huang, F. G. Omenetto, J. A. Rogers, A Physically Transient Form of Silicon Electronics. Science 337, 1640-1644 (2012).
[15]. Y. S. Oh, J.-H. Kim, Z. Xie, S. Cho, H. Han, S. W. Jeon, M. Park, M. Namkoong, R. Avila, Z. Song, S.-U. Lee, K. Ko, J. Lee, J.-S. Lee, W. G. Min, B.-J. Lee, M. Choi, H. U. Chung, J. Kim, M. Han, J. Koo, Y. S. Choi, S. S. Kwak, S. B. Kim, J. Kim, J. Choi, C.-M. Kang, J. U. Kim, K. Kwon, S. M. Won, J. M. Baek, Y. Lee, S. Y. Kim, W. Lu, A. Vazquez-Guardado, H. Jeong, H. Ryu, G. Lee, K. Kim, S. Kim, M. S. Kim, J. Choi, D. Y. Choi, Q. Yang, H. Zhao, W. Bai, H. Jang, Y. Yu, J. Lim, X. Guo, B. H. Kim, S. Jeon, C. Davies, A. Banks, H. J. Sung, Y. Huang, I. Park, J. A. Rogers, Battery-free, wireless soft sensors for continuous multi-site measurements of pressure and temperature from patients at risk for pressure injuries. Nat Commun 12, 5008 (2021).
[16]. D. R. Agrawal, Y. Tanabe, D. Weng, A. Ma, S. Hsu, S.-Y. Liao, Z. Zhen, Z.-Y. Zhu, C. Sun, Z. Dong, F. Yang, H. F. Tse, A. S. Y. Poon, J. S. Ho, Conformal phased surfaces for wireless powering of bioelectronic microdevices. Nat Biomed Eng 1, 1-9 (2017).
[17]. V. B. Koman, N. A. Bakh, X. Jin, F. T. Nguyen, M. Son, D. Kozawa, M. A. Lee, G. Bisker, J. Dong, M. S. Strano, A wavelength-induced frequency filtering method for fluorescent nanosensors in vivo. Nat. Nanotechnol. 17, 643-652 (2022).
[18]. S. Iwano, M. Sugiyama, H. Hama, A. Watakabe, N. Hasegawa, T. Kuchimaru, K. Z. Tanaka, M. Takahashi, Y. Ishida, J. Hata, S. Shimozono, K. Namiki, T. Fukano, M. Kiyama, H. Okano, S. Kizaka-Kondoh, T. J. McHugh, T. Yamamori, H. Hioki, S. Maki, A. Miyawaki, Single-cell bioluminescence imaging of deep tissue in freely moving animals. Science 359, 935-939 (2018).
[19]. S. Sonmezoglu, J. R. Fineman, E. Maltepe, M. M. Maharbiz, Monitoring deep-tissue oxygenation with a millimeter-scale ultrasonic implant. Nat Biotechnol 39, 855-864 (2021).
[20]. S. Sharma, K. B. Ramadi, N. H. Poole, S. S. Srinivasan, K. Ishida, J. Kuosmanen, J. Jenkins, F. Aghlmand, M. B. Swift, M. G. Shapiro, G. Traverso, A. Emami, Location-aware ingestible microdevices for wireless monitoring of gastrointestinal dynamics. Nat Electron 6, 242-256 (2023).
[21]. A. Hai, V. C. Spanoudaki, B. B. Bartelle, A. Jasanoff, Wireless resonant circuits for the minimally invasive sensing of biophysical processes in magnetic resonance imaging. Nat Biomed Eng 3, 69-78 (2019).
[22]. V. Kalidasan, X. Yang, Z. Xiong, R. R. Li, H. Yao, H. Godaba, S. Obuobi, P. Singh, X. Guan, X. Tian, S. A. Kurt, Z. Li, D. Mukherjee, R. Rajarethinam, C. S. Chong, J.-W. Wang, P. L. R. Ee, W. Loke, B. C. K. Tee, J. Ouyang, C. J. Charles, J. S. Ho, Wirelessly operated bioelectronic sutures for the monitoring of deep surgical wounds. Nat Biomed Eng 5, 1217-1227 (2021).
[23]. C. Wang, B. Qi, M. Lin, Z. Zhang, M. Makihata, B. Liu, S. Zhou, Y. Huang, H. Hu, Y. Gu, Y. Chen, Y. Lei, T. Lee, S. Chien, K.-I. Jang, E. B. Kistler, S. Xu, Continuous monitoring of deep-tissue haemodynamics with stretchable ultrasonic phased arrays. Nat Biomed Eng 5, 749-758 (2021).
[24]. D. Maresca, A. Lakshmanan, M. Abedi, A. Bar-Zion, A. Farhadi, G. J. Lu, J. O. Szablowski, D. Wu, S. Yoo, M. G. Shapiro, Biomolecular Ultrasound and Sonogenetics. Annual Review of Chemical and Biomolecular Engineering 9, 229-252 (2018).
[25]. E. Mace, G. Montaldo, I. Cohen, M. Baulac, M. Fink, M. Tanter, Functional ultrasound imaging of the brain. Nat Methods 8, 662-664 (2011).
[26]. A. Dizeux, M. Gesnik, H. Ahnine, K. Blaize, F. Arcizet, S. Picaud, J.-A. Sahel, T. Deffieux, P. Pouget, M. Tanter, Functional ultrasound imaging of the brain reveals propagation of task-related brain activity in behaving primates. Nat Commun 10, 1400 (2019).
[27]. R. W. Bourdeau, A. Lee-Gosselin, A. Lakshmanan, A. Farhadi, S. R. Kumar, S. P. Nety, M. G. Shapiro, Acoustic reporter genes for noninvasive imaging of microorganisms in mammalian hosts. Nature 553, 86-90 (2018).
[28]. A. Farhadi, G. H. Ho, D. P. Sawyer, R. W. Bourdeau, M. G. Shapiro, Ultrasound imaging of gene expression in mammalian cells. Science 365, 1469-1475 (2019).
[29]. E. Girard, M. Messager, A. Sauvanet, S. Benoist, G. Piessen, J.-Y. Mabrut, C. Mariette, Anastomotic leakage after gastrointestinal surgery: Diagnosis and management. Journal of Visceral Surgery 151, 441-450 (2014).
[30]. L. Yin, H. Cheng, S. Mao, R. Haasch, Y. Liu, X. Xie, S.-W. Hwang, H. Jain, S.-K. Kang, Y. Su, R. Li, Y. Huang, J. A. Rogers, Dissolvable Metals for Transient Electronics. Advanced Functional Materials 24, 645-658 (2014).
[31]. A. Lendlein, A. M. Schmidt, R. Langer, AB-polymer networks based on oligo(c-caprolactone) segments showing shape-memory properties. Proceedings of the National Academy of Sciences 98, 842-847 (2001).
[32]. S. S. Halacheva, D. J. Adlam, E. K. Hendow, T. J. Freemont, J. Hoyland, B. R. Saunders, Injectable Biocompatible and Biodegradable pH-Responsive Hollow Particle Gels Containing Poly(acrylic acid): The Effect of Copolymer Composition on Gel Properties. Biomacromolecules 15, 1814-1827 (2014).
[33]. M. Changez, V. Koul, B. Krishna, A. K. Dinda, V. Choudhary, Studies on biodegradation and release of gentamicin sulphate from interpenetrating network hydrogels based on poly(acrylic acid) and gelatin: in vitro and in vivo. Biomaterials 25, 139-146 (2004).
[34]. M. J. Bruining, H. G. T. Blaauwgeers, R. Kuijer, Elisabeth Pels, R. M. M. A. Nuijts, L. H. Koole, Biodegradable three-dimensional networks of poly(dimethylamino ethyl methacrylate). Synthesis, characterization and in vitro studies of structural degradation and cytotoxicity. Biomaterials 21, 595-604 (2000).
[35]. N. Deirram, C. Zhang, S. S. Kermaniyan, A. P. R. Johnston, G. K. Such, pH-Responsive Polymer Nanoparticles for Drug Delivery. Macromolecular Rapid Communications 40, 1800917 (2019).
[36]. O. E. Philippova, D. Hourdet, R. Audebert, A. R. Khokhlov, pH-Responsive Gels of Hydrophobically Modified Poly(acrylic acid). Macromolecules 30, 8278-8285 (1997).
[37]. H. Zhang, M. Dehghany, Y. Hu, Kinetics of Polyelectrolyte Gels. Journal of Applied Mechanics 87 (2020).
[38]. W. Hong, X. Zhao, J. Zhou, Z. Suo, A theory of coupled diffusion and large deformation in polymeric gels. Journal of the Mechanics and Physics of Solids 56, 1779-1793 (2008).
[39]. N. M. Tole, W. I. and L. Technology, Basic Physics of Ultrasonographic Imaging (World Health Organization, 2005).
[40]. J. T. Ylitalo, H. Ermert, Ultrasound synthetic aperture imaging: monostatic approach. IEEE Transactions on Ultrasonics, Ferroelectrics, and Frequency Control 41, 333-339 (1994).
[41]. C. Wang, B. Qi, M. Lin, Z. Zhang, M. Makihata, B. Liu, S. Zhou, Y. Huang, H. Hu, Y. Gu, Y. Chen, Y. Lei, T. Lee, S. Chien, K.-I. Jang, E. B. Kistler, S. Xu, Continuous monitoring of deep-tissue haemodynamics with stretchable ultrasonic phased arrays. Nat Biomed Eng 5, 749-758 (2021).
[42]. E. L. McConnell, A. W. Basit, S. Murdan, Measurements of rat and mouse gastrointestinal pH, fluid and lymphoid tissue, and implications for in-vivo experiments. Journal of Pharmacy and Pharmacology 60, 63-70 (2008).
[43]. A. E. Hanbidge, D. Lynch, S. R. Wilson, US of the Peritoneum. RadioGraphics 23, 663-685 (2003).
[44]. D. Garcia, “Make the most of MUST, an open-source Matlab UltraSound Toolbox” in 2021 IEEE International Ultrasonics Symposium (IUS) (2021), pp. 1-4.
[45]. D. Garcia, SIMUS: An open-source simulator for medical ultrasound imaging. Part I: Theory & examples. Computer Methods and Programs in Biomedicine 218, 106726 (2022).
[46]. A. Cigier, F. Varray, D. Garcia, SIMUS: An open-source simulator for medical ultrasound imaging. Part II: Comparison with four simulators. Computer Methods and Programs in Biomedicine 220, 106774 (2022).
[47]. V. B. Morris, S. Nimbalkar, M. Younesi, P. McClellan, O. Akkus, Mechanical Properties, Cytocompatibility and Manufacturability of Chitosan:PEGDA Hybrid-Gel Scaffolds by Stereolithography. Ann Biomed Eng 45, 286-296 (2017).
[48]. C. G. Williams, A. N. Malik, T. K. Kim, P. N. Manson, J. H. Elisseeff, Variable cytocompatibility of six cell lines with photoinitiators used for polymerizing hydrogels and cell encapsulation. Biomaterials 26, 1211-1218 (2005).
[49]. N. M. Rahman, E. K. Mishra, H. E. Davies, R. J. O. Davies, Y. C. G. Lee, Clinically Important Factors Influencing the Diagnostic Measurement of Pleural Fluid pH and Glucose. Am J Respir Crit Care Med 178, 483-490 (2008).
[50]. N. Gitlin, J. L. Stauffer, R. C. Silvestri, The pH of Ascitic Fluid in the Diagnosis of Spontaneous Bacterial Peritonitis in Alcoholic Cirrhosis. Hepatology 2, 408S-411S (1982).
[51]. D. A. Hickman, C. L. Pawlowski, U. D. S. Sekhon, J. Marks, A. S. Gupta, Biomaterials and Advanced Technologies for Hemostatic Management of Bleeding. Advanced Materials 30, 1700859 (2018).
[52]. N. Bhattarai, H. R. Ramay, J. Gunn, F. A. Matsen, M. Zhang, PEG-grafted chitosan as an injectable thermosensitive hydrogel for sustained protein release. Journal of Controlled Release 103, 609-624 (2005).
[53]. Q. Yang, T. Wei, R. T. Yin, M. Wu, Y. Xu, J. Koo, Y. S. Choi, Z. Xie, S. W. Chen, I. Kandela, S. Yao, Y. Deng, R. Avila, T.-L. Liu, W. Bai, Y. Yang, M. Han, Q. Zhang, C. R. Haney, K. Benjamin Lee, K. Aras, T. Wang, M.-H. Seo, H. Luan, S. M. Lee, A. Brikha, N. Ghoreishi-Haack, L. Tran, I. Stepien, F. Aird, E. A. Waters, X. Yu, A. Banks, G. D. Trachiotis, J. M. Torkelson, Y. Huang, Y. Kozorovitskiy, I. R. Efimov, J. A. Rogers, Photocurable bioresorbable adhesives as functional interfaces between flexible bioelectronic devices and soft biological tissues. Nat. Mater. 20, 1559-1570 (2021).
[54]. J. Bruce, Z. H. Krukowski, G. Al-Khairy, E. M. Russell, K. G. M. Park, Systematic review of the definition and measurement of anastomotic leak after gastrointestinal surgery. British Journal of Surgery 88, 1157-1168 (2001).
[55]. B. U. Su'a, H. L. Mikaere, J. L. Rahiri, I. B. Bissett, A. G. Hill, Systematic review of the role of biomarkers in diagnosing anastomotic leakage following colorectal surgery. British Journal of Surgery 104, 503-512 (2017).
[56]. H. Zhang, X. Lin, S. Chin, M. W. Grinstaff, Synthesis and Characterization of Poly(glyceric Acid Carbonate). A Degradable Analogue of Poly(acrylic Acid). J. Am. Chem. Soc. 137, 12660-12666 (2015).
[57]. B. Reid, M. Gibson, A. Singh, J. Taube, C. Furlong, M. Murcia, J. Elisseeff, PEG hydrogel degradation and the role of the surrounding tissue environment. Journal of Tissue Engineering and Regenerative Medicine 9, 315-318 (2015).
[58]. M. B. Browning, S. N. Cereceres, P. T. Luong, E. M. Cosgriff-Hernandez, Determination of the in vivo degradation mechanism of PEGDA hydrogels. Journal of Biomedical Materials Research Part A 102, 4244-4251 (2014).
[59]. Y. Zhang, M. Zheng, T. Kissel, S. Agarwal, Design and Biophysical Characterization of Bioresponsive Degradable Poly(dimethylaminoethyl methacrylate) Based Polymers for In Vitro DNA Transfection. Biomacromolecules 13, 313-322 (2012).
[60]. V. San Miguel, A. J. Limer, D. M. Haddleton, F. Catalina, C. Peinado, Biodegradable and thermoresponsive micelles of triblock copolymers based on 2-(N,N-dimethylamino)ethyl methacrylate and F-caprolactone for controlled drug delivery. European Polymer Journal 44, 3853-3863 (2008).
[61]. A. W. Jackson, S. R. Mothe, P. Ang, L. R. Chennamaneni, A. M. V. Herk, P. Thoniyot, Backbone degradable poly(acrylic acid) analogue via radical ring-opening copolymerization and enhanced biodegradability. Chemosphere 293, 133487 (2022).
[62]. D. Das, P. Ghosh, S. Dhara, A. B. Panda, S. Pal, Dextrin and Poly(acrylic acid)-Based Biodegradable, Non-Cytotoxic, Chemically Cross-Linked Hydrogel for Sustained Release of Ornidazole and Ciprofloxacin. ACS Appl. Mater. Interfaces 7, 4791-4803 (2015).
[63]. I. Gaytin, M. Burelo, H. Loza-Tavera, Current status on the biodegradability of acrylic polymers: microorganisms, enzymes and metabolic pathways involved. Appl Microbiol Biotechnol 105, 991-1006 (2021).
[64]. Z. Stillman, B. M. Jarai, N. Raman, P. Patel, C. A. Fromen, Degradation profiles of poly(ethylene glycol)diacrylate (PEGDA)-based hydrogel nanoparticles. Polym. Chem. 11, 568-580 (2020).
[65]. J. L. Grace, J. X. Huang, S.-E. Cheah, N. P. Truong, M. A. Cooper, J. Li, T. P. Davis, J. F. Quinn, T. Velkov, M. R. Whittaker, Antibacterial low molecular weight cationic polymers: dissecting the contribution of hydrophobicity, chain length and charge to activity. RSC Adv. 6, 15469-15477 (2016).
[66]. J. Zhang, R. Xie, S.-B. Zhang, C.-J. Cheng, X.-J. Ju, L.-Y. Chu, Rapid pH/temperature-responsive cationic hydrogels with dual stimuli-sensitive grafted side chains. Polymer 50, 2516-2525 (2009).
[67]. R. Yoshida, K. Uchida, Y. Kaneko, K. Sakai, A. Kikuchi, Y. Sakurai, T. Okano, Comb-type grafted hydrogels with rapid deswelling response to temperature changes. Nature 374, 240-242 (1995).
[68]. M. Dehghany, H. Zhang, R. Naghdabadi, Y. Hu, A thermodynamically-consistent large deformation theory coupling photochemical reaction and electrochemistry for light-responsive gels. Journal of the Mechanics and Physics of Solids 116, 239-266 (2018).
[69]. M. Rubinstein, Polymer Physics (Oxford University Press, 2003).
[70]. A. P. Safronov, L. V. Adamova, A. S. Blokhina, I. A. Kamalov, P. A. Shabadrov, Flory-Huggins parameters for weakly crosslinked hydrogels of poly(acrylic acid) and poly(methacrylic acid) with various degrees of ionization. Polym. Sci. Ser. A 57, 33-42 (2015).
[71]. Y. Lai, Y. Hu, Probing the swelling-dependent mechanical and transport properties of polyacrylamide hydrogels through AFM-based dynamic nanoindentation. Soft Matter 14, 2619-2627(2018).
[72]. H. F. Helander, L. Fandriks, Surface area of the digestive tract—revisited. Scandinavian Journal of Gastroenterology 49, 681-689 (2014).
[73]. V. Mahadevan, Anatomy of the stomach. Surgery (Oxford) 32, 571-574 (2014).
[74]. H. A. Messal, S. Alt, R. M. M. Ferreira, C. Gribben, V. M.-Y. Wang, C. G. Cotoi, G. Salbreux, A. Behrens, Tissue curvature and apicobasal mechanical tension imbalance instruct cancer morphogenesis. Nature 566, 126-130 (2019).
[75]. R. K. Goyal, Y. Guo, H. Mashimo, Advances in the physiology of gastric emptying. Neurogastroenterology & Motility 31, e13546 (2019).
[76]. A. Geliebter, S. A. Hashim, Gastric capacity in normal, obese, and bulimic women. Physiology & Behavior 74, 743-746 (2001).
[77]. S. Delgado-aros, F. Cremonini, J. E. Castillo, H. J. Chial, D. D. Burton, I. Ferber, M. Camilleri, Independent influences of body mass and gastric volumes on satiation in humans. Gastroenterology 126, 432-440 (2004).

What is claimed is:

1. A tag for real-time monitoring of homeostasis in tissue of a subject, comprising:
a soft, shape-adaptive matrix member configured to be attached onto the tissue, wherein the soft, shape-adaptive matrix member undergoes dimensional changes upon interactions with its surrounding biofluid environment by amounts that depend on a local parameter of interest around the tissue; and
a plurality of markers embedded in the soft, shape-adaptive matrix member, such that the dimensional changes of the soft, shape-adaptive matrix member lead to changes in spacings between the markers by magnitudes that are quantitatively determinable by imaging.

2. The tag of claim 1, wherein the soft, shape-adaptive matrix member and the markers are configured to undergo dissolution reactions and hydrolytic chain scissions in biofluids, thereby leading to conversion into benign products over well-defined timescales.

3. The tag of claim 1, wherein the plurality of markers is symmetrically distributed in the soft, shape-adaptive matrix member.

4. The tag of claim 3, wherein the plurality of markers is circularly distributed in the soft, shape-adaptive matrix member, which allows ultrasonic, CT and/or MRI visualization in a manner that is independent of the orientation of the markers.

5. The tag of claim 4, wherein each marker comprises a indicator that is detectable in ultrasound, CT and/or MRI images.

6. The tag of claim 5, wherein each indicator comprises a disc of a bioresorbable metal air cavity selected to maximize the acoustic impedance mismatch with the surrounding biofluid environment to ensure strong contrast in ultrasound images.

7. The tag of claim 6, wherein the bioresorbable metal comprises Mg, Zn, Fe, W, or Mo.

8. The tag of claim 1, being a bioresorbable, shape-adaptive, ultrasound, CT and/or MRI readable materials structure (BioSUM).

9. The tag of claim 1, wherein the soft, shape-adaptive matrix member is formed of a stimulus-responsive material that is fine-tuned to respond to changes in the surrounding biofluid environment of the soft, shape-adaptive matrix member.

10. The tag of claim 9, wherein the changes in the surrounding biofluid environment are induced by homeostatic perturbations in the tissue.

11. The tag of claim 10, wherein the homeostatic perturbations are of gastric leaks, small intestinal leaks, and/or pancreatic leaks.

12. The tag of claim 11, wherein the stimulus-responsive material comprises a pH-responsive hydrogel, designed to maximize its response in relevant pH ranges while maintaining stable mechanical properties.

13. The tag of claim 12, wherein the pH-responsive hydrogel is designed to respond to different pH changes by using the protonation behavior of tertiary amine and carboxylic acid, such that a change in pH values of the surrounding biofluid environment results in swelling of the soft, shape-adaptive matrix member, which in turn causes the markers in the soft, shape-adaptive matrix member to predictably spread apart, which can be continuously monitored through ultrasound images.

14. The tag of claim 13, wherein the pH-responsive hydrogel is designed to responsive to pH values as low as 1.0 in an acidic condition for monitoring of the gastric leaks.

15. The tag of claim 14, wherein the pH-responsive hydrogel comprises poly[2-(dimethylamino)ethyl methacrylate-co-2-(diisopropylamino)ethyl methacrylate][p(DMAEMA-DPAEMA)] with a weight ratio of DMAEMA:DPAEMA being 7:3, with polyethylene glycol diacrylate (PEGDA) as a crosslinker.

16. The tag of claim 13, wherein the pH-responsive hydrogel is designed to responsive to pH values of about 6.8 in a condition that is only slightly lower than normal physiological levels of about 7.4, for monitoring of the small intestinal leaks.

17. The tag of claim 16, wherein the pH-responsive hydrogel comprises p(DMAEMA-DPAEMA) with a weight ratio of DMAEMA:DPAEMA being 9:1, with PEGDA as a crosslinker.

18. The tag of claim 13, wherein the pH-responsive hydrogel is designed to responsive to pH values larger than 7.4 in an alkaline condition for monitoring of the pancreatic leaks.

19. The tag of claim 18, wherein the pH-responsive hydrogel comprises poly(acrylic acid-butyl acrylate) [p(AAc-BA)] with PEGDA as a crosslinker.

20. The tag of claim 10, being usable for real-time monitoring of pH changes associated with anastomotic leakage following GI surgeries, pleural effusion, ascites and complex infections.

21. The tag of claim 9, wherein the stimulus-responsive material is designed to maximize its response to other forms of homeostatic dysregulation including brain hemorrhage, wherein the stimulus-responsive material incorporates a hemostatic agent binding with red blood cells and absorption of blood, which results in its expansion as the basis for ultrasound detection.

22. The tag of claim 9, wherein the stimulus-responsive material comprises a thermos-responsive material that incorporates polyethylene glycol grafted into a chitosan hydrogel, thereby enabling sensing of changes in temperature.

23. The tag of claim 9, wherein the stimulus-responsive material is designed to maximize its response to inflammation or flare-ups in bowels of people with irritable bowel syndrome, and/or sepsis.

24. The tag of claim 9, wherein the stimulus-responsive material is designed to maximize its response to biomolecules including drug metabolites or antibody accumulation, and/or pathogens including bacteria or viruses.

25. The tag of claim 24, being configured to real-time monitor the body's response to a drug, thereby eliminating the need for multiple invasive blood draws, and/or track the onset of infection while in resource-limited settings.

26. The tag of claim 1, wherein the soft, shape-adaptive matrix member has an anchor feature for suturing the soft, shape-adaptive matrix member to the tissue.

27. The tag of claim 1, wherein the soft, shape-adaptive matrix member is attached onto the tissue by bioresorbable adhesives.

28. The tag of claim 1, being implantable to the tissue.

29. The tag of claim 1, being injectable to the tissue.

30. The tag of claim 1, being bioresorbable and/or biodegradable.

31. The tag of claim 1, being usable for post-surgical monitoring of patient status throughout a recovery period, after which resorption into the body via hydrolysis and natural metabolic reactions naturally eliminates the tag, thereby eliminating the need for surgical extraction procedures subsequent to a desired timeframe for operation.

32. The tag of claim 1, being configured to incorporate ultrasound image processing software in the workflow, with automated feature detection and/or artificial intelligence, to allow the patient to detect postoperative complications and return to the hospital if needed.

33. A method of real-time monitoring of homeostasis in tissue of a subject, comprising:
attaching at least one tag onto at least one surface of the tissue, wherein the at least one tag comprises:
a soft, shape-adaptive matrix member that undergoes dimensional changes upon interactions with its surrounding biofluid environment by amounts that depend on a local parameter of interest around the tissue; and
a plurality of markers embedded in the soft, shape-adaptive matrix member such that the dimensional changes of the soft, shape-adaptive matrix member lead to changes in spacings between the markers;
acquiring images of the plurality of markers; and
determining the changes in the spacings between the markers from the acquired images, thereby determining changes in the surrounding biofluid environment and real-time monitoring of homeostasis in the tissue.

34. The method of claim 33, wherein the plurality of markers is symmetrically distributed in the soft, shape-adaptive matrix member.

35. The method of claim 34, wherein the plurality of markers is circularly distributed in the soft, shape-adaptive matrix member, which allows ultrasonic, CT and/or MRI visualization in a manner that is independent of the orientation of the markers.

36. The method of claim 35, wherein each marker comprises a indicator that is detectable in ultrasound, CT and/or MRI images.

37. The method of claim 36, wherein each indicator comprises a disc of a bioresorbable metal air cavity selected to maximize the acoustic impedance mismatch with the surrounding biofluid environment to ensure strong contrast in ultrasound images.

38. The method of claim 37, wherein the bioresorbable metal comprises Mg, Zn, Fe, W, or Mo.

39. The method of claim 33, wherein the tag is of a bioresorbable, shape-adaptive, ultrasound, CT and/or MRI readable materials structure (BioSUM).

40. The method of claim 33, wherein the soft, shape-adaptive matrix member is formed of a stimulus-responsive material that is fine-tuned to respond to changes in the surrounding biofluid environment of the soft, shape-adaptive matrix member.

41. The method of claim 40, wherein the changes in the surrounding biofluid environment are induced by homeostatic perturbations in the tissue.

42. The method of claim 41, wherein the homeostatic perturbations are of gastric leaks, small intestinal leaks, and/or pancreatic leaks.

43. The method of claim 42, wherein the stimulus-responsive material comprises a pH-responsive hydrogel, designed to maximize its response in relevant pH ranges while maintaining stable mechanical properties.

44. The method of claim 43, wherein the pH-responsive hydrogel is designed to respond to different pH changes by using the protonation behavior of tertiary amine and carboxylic acid, such that a change in pH values of the surrounding biofluid environment results in swelling of the soft, shape-adaptive matrix member, which in turn causes the markers in the soft, shape-adaptive matrix member to predictably spread apart, which can be continuously monitored through ultrasound images.

45. The method of claim 44, wherein the pH-responsive hydrogel is designed to responsive to pH values as low as 1.0 in an acidic condition for monitoring of the gastric leaks.

46. The method of claim 45, wherein the pH-responsive hydrogel is synthesized by performing a chain extension reaction of a mixture of 2-(dimethylamino)ethyl methacrylate monomer (DMAEMA) and 2-(diisopropylamino)ethyl methacrylate monomer (DPAEMA) with a weight ration of DMAEMA:DPAEMA being about 7:3;
adding about 2 wt % 2-hydroxy-2-methylpropiophenone photoinitiator, followed by UV irradiation to yield a monomer mixture;
subsequently adding about 3.85 wt % poly(ethylene glycol) diacrylate (PEGDA) and about 0.5 wt % 2,2-dimethoxy-2-phenylacetophenone photoinitiator (DMPA) to the monomer mixture to form a hydrogel precursor; and
UV exposing to the hydrogel precursor under a nitrogen atmosphere to yield a crosslinked hydrogel network.

47. The method of claim 44, wherein the pH-responsive hydrogel is designed to responsive to pH values of about 6.8 in a condition that is only slightly lower than normal physiological levels of about 7.4, for monitoring of the small intestinal leaks.

48. The method of claim 47, wherein the pH-responsive hydrogel is synthesized by performing a chain extension reaction of a mixture of DMAEMA and DPAEMA with a weight ration of DMAEMA:DPAEMA being about 9:1;
adding about 2 wt % 2-hydroxy-2-methylpropiophenone photoinitiator, followed by UV irradiation to yield a monomer mixture;
subsequently adding about 3.85 wt % PEGDA and about 0.5 wt % DMPA to the monomer mixture to form a hydrogel precursor; and
UV exposing to the hydrogel precursor under a nitrogen atmosphere to yield a crosslinked hydrogel network.

49. The method of claim 44, wherein the pH-responsive hydrogel is designed to responsive to pH values larger than 7.4 in an alkaline condition for monitoring of the pancreatic leaks.

50. The method of claim 49, wherein the pH-responsive hydrogel is synthesized by performing a chain extension reaction of butyl acrylate monomer (BA);
adding about 0.5 wt % 2-hydroxy-2-methylpropiophenone photoinitiator, followed by UV irradiation to yield a BA oligomer;
subsequently adding about 21.8 wt % of a pH-responsive monomer acrylic acid (AAc), about 1 wt % PEGDA and about 0.5 wt % DMPA photoinitiator to a BA oligomer to form a hydrogel precursor to the hydrogel; and
UV exposing to the hydrogel precursor under a nitrogen atmosphere to yield a crosslinked hydrogel network.

51. The method of claim 40, being usable for real-time monitoring of pH changes associated with anastomotic leakage following GI surgeries, pleural effusion, ascites and complex infections.

52. The method of claim 40, wherein the stimulus-responsive material is designed to maximize its response to other forms of homeostatic dysregulation including brain hemorrhage, wherein the stimulus-responsive material incorporates a hemostatic agent binding with red blood cells and absorption of blood, which results in its expansion as the basis for ultrasound detection.

53. The method of claim 40, wherein the stimulus-responsive material comprises a thermos-responsive material that incorporates polyethylene glycol grafted into a chitosan hydrogel, thereby enabling sensing of changes in temperature.

54. The method of claim 40, wherein the stimulus-responsive material is designed to maximize its response to inflammation or flare-ups in bowels of people with irritable bowel syndrome, and/or sepsis.

55. The method of claim 40, wherein the stimulus-responsive material is designed to maximize its response to biomolecules including drug metabolites or antibody accumulation, and/or pathogens including bacteria or viruses.

56. The method of claim 55, being usable for real-time monitoring of the body's response to a drug, thereby eliminating the need for multiple invasive blood draws, and/or track the onset of infection while in resource-limited settings.

57. The method of claim 33, wherein said attaching at least one tag comprises one of:
suturing the soft, shape-adaptive matrix member to the tissue;
adhering the soft, shape-adaptive matrix member to the tissue; and
injecting the tag, thus the soft, shape-adaptive matrix member to the tissue.

58. The method of claim 57 wherein said adhering the soft, shape-adaptive matrix member to the tissue is formed with a photo-triggered liquid-solid transformation of the bioresorbable adhesive that enables the formation of bonding without applying external forces, thereby facilitating the attachment procedure, and minimizing the potential damage to the tissues.

59. The method of claim 58, wherein said adhering the soft, shape-adaptive matrix member to the tissue comprises:
coating the tissue surface with a primer layer including chitosan, sulfated N-hydroxysuccinimide (Sulfo-NHS) and (1-ethyl-3-(3-dimethylaminopropyl) carbodiimide (EDC), followed by applying a two-component bioresorbable adhesive onto the tag and the tissue surface, wherein the two-component adhesive is a viscous liquid solution of a photocurable covalent network of polyethylene glycol-lactide acid diacrylate (PEG-LA-DA) and an ionic network of sodium alginate; and
exposing them to UV light to complete the attachment of the tag onto the tissue surface with robust chemical bonding.

60. The method of claim 33, wherein said attaching at least one tag comprises attaching multiple tags at strategic anatomical locations relative to a surgical site provide the basis for spatio-temporal monitoring of homeostasis.
